(19) United States
(12) Patent Application Publication
(10) Pub. No.: US 2004/0197892 A1
(43) Pub. Date: Oct. 7, 2004

(54) Composition binding polypeptides

(76) Inventors: Michael Moore, Bucks (GB); Armin Sepp, Cambridge (GB); Mark Isalan, London (GB); Yen Choo, London (GB)

Correspondence Address: ROBINS & PASTERNAK, 1731 EMBARCADERO ROAD, SUITE 230, PALO ALTO, CA, 94303, US

(21) Appl. No.: 10/474,282

(22) PCT Filed: Apr. 4, 2002
(86) PCT No.: PCT/US02/22272

(30) Foreign Application Priority Data
Apr. 4, 2001 (GB) 0108491.2

(51) Int. Cl. C12N 9/64

(52) U.S. Cl. 435/226

(57) ABSTRACT

Disclosed herein are polypeptides with novel DNA binding specificities, constructed from combinations of zinc fingers, and methods for their preparation and use.

### TECHNICAL FIELD

The present disclosure is in the fields of molecular biology and protein design; in particular, the design of sequence-specific binding proteins for regulation of gene expression.

### BACKGROUND

Protein-nucleic acid recognition is a commonplace phenomenon that is central to a large number of biomolecular control mechanisms that regulate the functioning of eukaryotic and prokaryotic cells. For instance, protein-DNA interactions form the basis of the regulation of gene expression and are thus one of the subjects most widely studied by molecular biologists.

A wealth of biochemical and structural information explains the details of protein-DNA recognition in numerous instances, to the extent that general principles of recognition have emerged. Many DNA-binding proteins contain independently folded domains for the recognition of DNA, and these domains in turn belong to a large number of structural families, such as the leucine zipper, the “helix-turn-helix” and zinc finger families.

Despite the great variety of structural domains, the specificity of the interactions observed to date between protein and DNA most often derives from the complementarity of the surfaces of a protein a-helix and the major groove of DNA. See, e.g., Klug, (1993) Gene 135:83-92. In light of the recurring physical interaction of a-helix and major groove, the tantalising possibility arises that the contacts between particular amino acids and DNA bases could be described by a simple set of rules; in effect a stereochemical recognition code which relates protein primary structure to binding-site sequence preference.

It is clear, however, that no code will be found which can describe DNA recognition by all DNA-binding proteins. The structures of numerous complexes show significant differences in the way that the recognition α-helices of DNA-binding proteins from different structural families interact with the major groove of DNA, thus precluding similarities in patterns of recognition. The majority of known DNA-binding motifs are not particularly versatile, and any codes which might emerge would likely describe binding to a very few related DNA sequences.

Even within each family of DNA-binding proteins, moreover, it has hitherto appeared that the deciphering of a code would be elusive. Due to the complexity of the protein-DNA interaction, there does not appear to be a simple “alphabetic” equivalence between the primary structures of protein and nucleic acid which specifies a direct amino acid to base relationship.

International patent application WO 96/06166 addresses this issue and provides a “syllabic” code that explains protein-DNA interactions for zinc finger nucleic acid binding proteins. A syllabic code is a code that relies on more than one feature of the binding protein to specify binding to a particular base, the features being combinable in the forms of “syllables”, or complex instructions, to define each specific contact. Segal, D. J., Dreier, B., Beerli, R. R. & Barbas, C. F. (1999) Proc. Natl. Acad. Sci. USA 96, 2758-2763 present a method of constructing zinc fingers polypeptides, based on 16 individual zinc finger domains which bind sequences of the form 5′-GXX-3′, where X is any base. See also U.S. Pat. No. 6,140,081. The latter method has the severe limitation that it does not provide instructions permitting the specific targeting of triplets containing nucleotides other than G in the 5′ position of each triplet, which greatly restricts the potential target sequences of such generated zinc finger peptides.

International patent application WO98/53057 addresses the above problems by recognizing that zinc fingers can specify overlapping 4 bp subsites, and therefore synergy between adjacent zinc finger domains is an important consideration in selecting zinc finger nucleic acid-binding domains to specifically target any sequence.

With the recent completion of the human genome project and the rapidly advancing fields of transgenic animals and plants, thousands of uncharacterised (and characterised) genes have (and will) become valid targets for functional genomics and other such projects. Concomitantly, ‘designer’ zinc finger peptides are emerging as one of the most universal and desirable ways of regulating the expression of specific genes within cells. See, for example, Choo, Y., Sanchez-Garcia, I. & Klug, A. (1994) Nature 372: 642-645; Beerli, R. R., Dreier, B. & Barbas, C. F. III (2000) Proc. Natl. Acad. Sci. USA 97: 1495-1500; Kim, J-S. & Pabo, C. O. (1998) Proc. Natl. Acad. Sci. USA 95: 2812-2817; Kang, J. S. & Kim, J-S. (2000) J. Biol. Chem. 275: 8742-8748); Zhang et al. (2000) J. Biol. Chem. 275:33,850-33,860; Liu et al. (2001) J. Biol. Chem. 276:11,323-11,334; and Ren et al. (2002) Genes. Devel.16:27-32. See also WO 00/41566 and WO 01/19981. Hence, a rapid method of creating multi-zinc finger peptides for the up- or down-regulation of any specific gene is highly desirable.

As stated above, synergy between adjacent zinc finger peptides is an important factor in specific DNA recognition. Moreover, the findings reported in co-owned WO 01/53480, which is hereby incorporated by reference, demonstrate that poly-zinc finger peptides constructed from strings of 2-finger domains can provide greater DNA binding specificity.

Traditional strategies of zinc finger mutagenesis and selection, such as phage display, particularly if employed for the selection of 2-zinc finger units to target any desired binding site are limited by the size of the library that can be cloned into host/vector systems, such as phage. Due to limitations in library size imposed by such constraints, it is impossible to include an exhaustive combination of randomisations to cover all potentially important sequence-space. Furthermore, for important applications of engineered zinc finger peptides, such as for gene therapy or transgenic animal systems, engineered zinc finger peptides run the significant risk of eliciting a harmful immunological reaction in the host animal.

The human genome sequencing project has also revealed the presence of almost 700 endogenous zinc finger-containing proteins. Assuming that each of these proteins contains at least 2 finger modules, there are probably at least 2,000 natural zinc finger modules in the human genome alone. Similar numbers are expected in other animal and plant genomes.

### SUMMARY

The present invention recognises the potential importance of designer zinc finger peptides in therapeutic and transgenic applications in animals and plants. Furthermore the present invention acknowledges that the safety of such applications is of primary importance.

The present invention provides the isolation of natural zinc finger modules, from genomes such as human, mouse, chicken, arabidopsis and other species, and the construction of non-natural combinations of such zinc finger modules, to create multi-finger domains, and to provide and determine novel nucleic acid binding specificities. Such a procedure will allow the identification of the novel zinc finger domains that bind any desired nucleic acid sequence, particularly sequences of between 6 and 10 nucleotides long. The first advantage of such technology is that millions of years of natural evolution, to create specific nucleotide-binding zinc finger modules, are captured to create novel nucleic acid-binding domains. Also, use of poly-zinc finger peptides constructed from such units for targeted gene regulation avoids the potentially harmful effects of host immune responses. The present invention thus greatly enhances the possibilities for the use of zinc finger transcription factors for in vivo applications, such as gene therapy and transgenic animals.

In a first aspect, therefore, there is provided a composite binding polypeptide comprising a first natural binding domain derived from first natural binding polypeptide, and a second natural binding domain derived from a second natural binding polypeptide, wherein said first and second natural binding polypeptides may be the same or different; which polypeptide binds to a target, said target differing from the natural target of the both the first and the second binding polypeptides.

Preferably, said first and second natural binding polypeptides are different polypeptides.

Binding polypeptides according to the invention comprise two or more natural binding domains, advantageously three or more natural binding domains; advantageously, six or more domains are included. These are preferably arranged in a 3×2 conformation, separated by linker sequences.

The binding domains are preferably nucleic acid binding domains, and the composite polypeptide is preferably a nucleic acid binding polypeptide. Most preferably, the composite polypeptide is a zinc finger polypeptide, and the natural binding domains are zinc finger domains.

Zinc finger binding domains can comprise any type of zinc finger or zinc-coordinated structure including, but not limited to, Cys2-His2 (SEQ ID NO:1) zinc finger binding domain or Cys3-His (SEQ ID NO:2) zinc finger binding domains.

In a further aspect, there is provided a library of natural binding domains. The natural binding domains are the domains that may be assembled into polypeptides according to the previous aspect of the invention. Preferably, the library is of natural zinc finger nucleic acid binding domains.

Said zinc finger domains may comprise a linker attached thereto. Any linker amino acid sequence known in the art can be used. Advantageously, the linker comprises the amino acid sequence TGEKP (SEQ ID NO:3).

In a further aspect, the invention provides a method for selecting a binding polypeptide capable of binding to a target site, comprising:

(a) providing a library of natural binding domains;

(b) assembling two or more of said domains to form a composite polypeptide;

(c) screening said composite polypeptide against the target site in order to determine its ability to bind the target site.

Preferably, the natural binding domains are zinc finger binding domains.

Furthermore, the invention provides methods for designing a composite binding polypeptide, comprising:

(a) providing information defining a target site;

(b) selecting, from a database of natural binding domains, a sequence of binding domains, separated by linker sequences, which is predicted to bind to the target site;

(c) displaying the sequence of binding domains and linkers and optionally assembling the binding polypeptide from a library of said domains.

In certain embodiments, the binding domains are zinc finger domains. In certain embodiments, a binding domain sequence that will bind a particular target site is predicted by the application of one or more rules that define target binding interactions for the binding domains. In additional embodiments, a nucleotide sequence encoding the binding domains is assembled and introduced into a cell such that the composite binding polypeptide is expressed.

In one embodiment, zinc fingers can be considered to bind to a nucleic acid triplet, in which case domains can be selected according to one or more of the following rules:

(a) if the 5′ base in the triplet is G, then position +6 in the α-helix is Arg; or position +6 is Ser or Thr and position ++2 is Asp;

(b) if the 5′ base in the triplet is A, then position +6 in the α-helix is Gln and ++2 is not Asp;

(c) if the 5′ base in the triplet is T, then position +6 in the α-helix is Ser or Thr and position ++2 is Asp;

(d) if the 5′ base in the triplet is C, then position +6 in the α-helix may be any amino acid, provided that position ++2 in the α-helix is not Asp;

(e) if the central base in the triplet is G, then position +3 in the α-helix is His;

(f) if the central base in the triplet is A, then position +3 in the α-helix is Asn;

(g) if the central base in the triplet is T, then position +3 in the α-helix is Ala, Ser or Val; provided that if it is Ala, then one of the residues at −1 or +6 is a small residue;

(h) if the central base in the triplet is C, then position +3 in the α-helix is Ser, Asp, Glu, Leu, Thr or Val;

(i) if the 3′ base in the triplet is G, then position −1 in the a:-helix is Arg;

(j) if the 3′ base in the triplet is A, then position -I in the α-helix is Gln;

(k) if the 3′ base in the triplet is T, then position −1 in the α-helix is Asn or Gln;

(l) if the 3′ base in the triplet is C, then position -I in the α-helix is Asp. In a further embodiment, the zinc fingers can be considered to bind to a nucleic acid quadruplet and domains can be selected according to one or more of the following rules:

(a) if base 4 in the quadruplet is G, then position +6 in the α-helix is Arg or Lys;

(b) if base 4 in the quadruplet is A, then position +6 in the α-helix is Glu, Asn or Val;

(c) if base 4 in the quadruplet is T, then position +6 in the α-helix is Ser, Thr, Val or Lys;

(d) if base 4 in the quadruplet is C, then position +6 in the α-helix is Ser, Thr, Val, Ala, Glu or Asn;

(e) if base 3 in the quadruplet is G, then position +3 in the α-helix is His;

(f) if base 3 in the quadruplet is A, then position +3 in the ax-helix is Asn;

(g) if base 3 in the quadruplet is T, then position +3 in the α-helix is Ala, Ser or Val; provided that if it is Ala, then one of the residues at −1 or +6 is a small residue;

(h) if base 3 in the quadruplet is C, then position +3 in the α-helix is Ser, Asp, Glu, Leu, Thr or Val;

(i) if base 2 in the quadruplet is G, then position −1 in the α-helix is Arg;

(j) if base 2 in the quadruplet is A, then position −1 in the α-helix is Gln;

(k) if base 2 in the quadruplet is T, then position −1 in the α-helix is His or Thr;

(l) if base 2 in the quadruplet is C, then position −1 in the α-helix is Asp or His;

(m) if base 1 in the quadruplet is G, then position +2 is Glu;

(n) if base 1 in the quadruplet is A, then position +2 Arg or Gln;

(o) if base 1 in the quadruplet is C, then position +2 is Asn, Gln, Arg, His or Lys;

(p) if base 1 in the quadruplet is T, then position +2 is Ser or Thr.

In a preferred embodiment, zinc fingers are considered to bind to a nucleic acid quadruplet and domains are selected according to one or more of the following rules:

(a) if base 4 in the quadruplet is G, then position +6 in the α-helix is Arg; or position +6 is Ser or Thr and position ++2 is Asp;

(b) if base 4 in the quadruplet is A, then position +6 in the α-helix is Gln and ++2 is not Asp;

(c) if base 4 in the quadruplet is T, then position +6 in the α-helix is Ser or Thr and position ++2 is Asp;

(d) if base 4 in the quadruplet is C, then position +6 in the α-helix may be any amino acid, provided that position ++2 in the α-helix is not Asp;

(e) if base 3 in the quadruplet is G, then position +3 in the α-helix is His;

(f) if base 3 in the quadruplet is A, then position +3 in the α-helix is Asn;

(g) if base 3 in the quadruplet is T, then position +3 in the α-helix is Ala, Ser or Val; provided that if it is Ala, then one of the residues at −1 or +6 is a small residue;

(h) if base 3 in the quadruplet is C, then position +3 in the α-helix is Ser, Asp, Glu, Leu, Thr or Val;

(i) if base 2 in the quadruplet is G, then position −1 in the α-helix is Arg;

(j) if base 2 in the quadruplet is A, then position −1 in the α-helix is Gln;

(k) if base 2 in the quadruplet is T, then position −1 in the α-helix is Asn or Gln;

(l) if base 2 in the quadruplet is C, then position −1 in the α-helix is Asp;

(m) if base 1 in the quadruplet is G, then position +2 is Asp;

(n) if base 1 in the quadruplet is A, then position +2 is not Asp;

(o) if base 1 in the quadruplet is C, then position +2 is not Asp;

(p) if base 1 in the quadruplet is T, then position +2 is Ser or Thr.

Two or more composite polypeptides comprising two or more domains which are selected for binding to two or more target sites can be combined to provide a composite polypeptide which binds to an aggregate binding site comprising the two or more target binding sites.

In a still further aspect, the invention provides a computer-implemented method for designing a zinc finger polypeptide that binds to a target nucleic acid sequence, comprising the steps of:

(a) providing a system comprising at least storage means for storing data relating to a library of zinc fingers; storage means for storing a rule table; means for inputting target nucleic acid sequence data; processing means for generating a result; and means for outputting the result;

(b) inputing sequence data for a target nucleic acid molecule;

(c) defining a first target zinc finger binding site in said nucleic acid molecule;

(d) interrogating the zinc finger library and rule table storage means, comparing zinc fingers to the target zinc finger binding site according to the rule table and selecting zinc finger data identifying a zinc finger capable of binding to said target site;

(e) defining at least one further target zinc finger binding site and repeating step (d); and

(f) outputting the selected zinc finger data.

Such a method may further comprise sending instructions to an automated chemical synthesis system to assemble a zinc finger polypeptide as defined by the zinc finger data obtained in (f).

In additional embodiments, the sequence of one or more oligonucleotides encoding a composite binding polypeptide can be determined from the sequence of a composite binding polypeptide, and the one or more oligonucleotides can be synthesized by any number of well-known methods.

Preferably, a composite binding polypeptide is tested for binding to a target sequence, and data from said testing is used to select, from a plurality of possibilities, a composite binding polypeptide that binds with optimal affinity and specificity to the target site.

Advantageously, two or more zinc finger polypeptides are combined to form a zinc finger polypeptide capable of binding to an aggregate binding site comprising two or more target sites.

The rule table preferably comprises rules as set forth above.

### BRIEF DESCRIPTION OF THE FIGURES

FIG. 1 shows a flowchart depicting part of the logic used in the selection of zinc fingers from a natural library in accordance with the invention. The logic set forth in FIG. 1 may be supplemented, for example using Rules relating to zinc finger overlap. Functional testing of zinc fingers for binding to the desired binding site may be implemented in an automated fashion and integrated with the zinc finger design system.

FIG. 2 is a schematic representation of the human zinc finger mini-library construction procedure. Synthetic zinc finger coding oligonucleotides are assembled into full-length ds expression constructs by overlap PCR.

FIG. 3 is a schematic representation of the fluorescent ELISA assay used to detect zinc finger peptides bound to double stranded DNA target sites. Streptavidin (7), biotinylated DNA target (5) linked to biotin (6), 3-finger peptide (4) fused to HA-tag (3), anti-HA antibody (2) fused to horseradish peroxidase (HRP, 1).

FIG. 4 depicts ELISA scores of 384 library 2 constructs screened against the 5′-GCG-TG-GCG-3′ (SEQ ID NO:4) target site. Six constructs showed significant binding, and are termed C8, G16, I19, I23, J19 and K19, according to their coordinates on the 384-well plate.

FIG. 5 depicts ELISA scores of selected library 2 members; B10, C8, G16, I23, J19, and K19, against different DNA target sites. The sequences of the target sites are (from back of graph to front): 5′-GCG-TGG-GCG-3′ (SEQ ID NO:5); 5′-CCA-CTC-GGC-3′ (SEQ ID NO:6); 5′-CCT-AGG-GGG-3′ (SEQ ID NO:7); 5′-GGA-TAA-GCG-3′ (SEQ ID NO:8); 5′-GGG-AGG-CCT-3′ (SEQ ID NO:9); 5′-GCG-TAA-GGA-3′ (SEQ ID NO:10); 5′-GCG-GGG-GGA-3′ (SEQ ID NO:11); and no DNA control (front row).

FIG. 6 depicts a schematic representation of the 3-zinc finger library constructed according to the procedure described in Example 2.

### DETAILED DESCRIPTION

Definitions

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). The practice of the present invention will employ, unless otherwise indicated, conventional techniques of chemistry, molecular biology, microbiology, recombinant DNA, immunology, chemical methods, pharmaceutical formulations and delivery and treatment of patients, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice; Oxford University Press; M. J. Gait (Editor), 1984, Oligonucleotide Synthesis: A Practical Approach, IRL Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The term “library” is used according to its common usage in the art, to denote a collection of different polypeptides or, preferably, a collection of nucleic acids encoding different polypeptides. The libraries of natural zinc finger peptides referred to herein comprise or encode a repertoire of polypeptides of different sequences, each of which has a preferred binding sequence.

The terms “polypeptide”, “peptide” and “protein” are used interchangeably to refer to a polymer of amino acid residues, preferably including naturally occurring amino acid residues. Artificial amino acid residues are also within the scope of the invention, but the exclusive use of naturally-occurring amino acids is preferred in order to maintain the natural nature of the binding domains. There are 20 common amino acids, each specified by a different arrangement of three adjacent DNA nucleotides by the genetic code. These are the building blocks of proteins. Joined together in a strictly ordered chain by peptide bonds, the sequence of amino acids determines each polypeptide molecule. The 20 common amino acids are: alanine, arginine, aspartic acid, glutamic acid, glutamine, glycine, histidine, isoleucine, leucine, phenylalanine, proline, serine, threonine, tryptophan, tyrosine, valine, cysteine, methionine, lysine, and asparagine. Virtually all of these amino acids (except glycine) possess an asymmetric carbon atom, and thus are potentially chiral in nature.

As used herein, “nucleic acid” includes both RNA and DNA, and nucleic acids constructed from natural nucleic acid bases or synthetic bases, or mixtures thereof. Modified nucleic acids such as, for example, PNAs and morpholino nucleic acids, are also included in this definition.

A “gene”, as used herein, is the segment of nucleic acid (typically DNA) that is involved, in producing a polypeptide chain or ribonucleic acid gene product. It includes regions preceding and following the coding region (leader and trailer) as well as intervening sequences (introns) between individual coding segments (exons). Preferably, “gene” includes the necessary control sequences for gene expression, as well as the coding region encoding the gene product.

A “binding polypeptide” is a polypeptide capable of binding to a specific target. Although, as is well known, polypeptides are capable of non-specific binding to a wide range of substrates, it is also known that certain polypeptides, such as antibodies and other members of the immunoglobulin superfamily, zinc fingers, leucine zipper polypeptides, peptide aptamers and the like can bind specifically to target sites or molecules. Generally, specific binding is preferably achieved with a dissociation constant (K_{d}) of 100 μM or lower; preferably 10 μM or better; preferably l1 M or better; and ideally 0.5 μM or better. Binding polypeptides can be nucleic acid binding polypeptides which bind to nucleic acid in a target sequence-specific manner, such as zinc finger polypeptides. Unless specifically noted, no difference is intended herein between terms such as “peptide”, “polypeptide” and “protein”.

A “natural binding polypeptide” is a binding polypeptide encoded by the genome of a living organism such as, for example, a plant or animal.

A “composite” polypeptide is a polypeptide that is assembled from a plurality of components. In a preferred embodiment, the invention provides composite binding polypeptides that are assembled from a plurality of individual natural binding domains as set forth in detail herein. Typically, such domains are zinc finger nucleic acid binding domains.

A “natural binding domain” (or module) is a domain of a naturally occurring polypeptide that is capable of specific binding to a target as defined above. The terms “domain” and “module”, according to their ordinary signification in the art, refer to a discrete continuous part of the amino acid sequence of a polypeptide that can be equated with a particular function. Protein domains or modules are largely structurally independent and can retain their structure and function in different environments. In certain embodiments, a natural binding domain or module is a zinc finger that binds a triplet or quadruplet nucleotide sequence.

Preferably, each of the individual natural binding domains that make up a composite binding polypeptide contain no changes in sequence, as compared to the natural sequence. However, those skilled in the art will understand that certain changes including conservative amino acid substitutions, as well as additions or deletions, may be made 30 without altering the function of a domain. Moreover, where the changes are consistent with sequences common to the species from which the domain is derived, such as for example being present in consensus sequences, they are unlikely to give rise to immunological problems.

Conservative amino acid substitutions may be made, for example according to Table 1. Amino acids in the same block in the second column and preferably in the same line in the third column may be substituted for one another:

TABLE 1

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

A domain is “derived” from a protein if it is effectively removed from a naturally-occurring protein for use in a composite binding polypeptide. Removal may be physical removal, by cleavage of the protein; more commonly, however, the sequence of the domain is determined and the domain is synthesised by protein synthesis techniques to be a copy of the naturally-occurring domain. Alternatively, a nucleic acid encoding the domain is synthesized and expressed in a cell. In vitro synthesised domains, or in vitro synthesized polynucleotides encoding naturally-occurring domains, are considered to be “derived” from the natural protein if they recapitulate the sequence of the naturally-occurring domain.

A “target” is a molecule or part thereof to which a binding polypeptide or a binding domain is capable of specific binding. The “natural target” of a binding polypeptide is the target to which that polypeptide binds in nature; e.g., in a living cell. In the case of zinc finger polypeptides, for instance, the natural target is the nucleotide sequence to which the polypeptide binds in a living cell. Sequences other than the natural target, as defined herein, to which a zinc finger polypeptide may bind in vitro are not natural targets.

In the case of nucleic acid binding polypeptides, therefore, the term “target” may be substituted or supplemented with “binding site” or “binding sequence.” Where binding sites are assembled to form larger binding sites, which are bound by multi-domain binding polypeptides, such binding sites are referred to as “aggregate binding sites”, indicating that they are formed by the juxtaposition of two or more individual binding sites. The aggregate binding sites can comprise contiguous individual binding sites, or individual binding sites interspersed by one or more intervening nucleotides or sequence of nucleotides.

The present invention relates to naturally-occurring zinc fingers and their use as specific nucleic acid binding modules in combinations not present in nature. This invention provides methods of determining and/or predicting the nucleotide binding specificities of natural zinc finger modules. Also provided are methods of constructing poly-zinc finger peptides containing at least one natural zinc finger module, from libraries of natural zinc finger peptides, and methods of screening such peptides to determine their preferred nucleotide binding specificity. Moreover, the invention provides for the use of combinations of such natural zinc finger modules in poly-zinc finger peptides not present in nature, to bind any desired nucleotide sequence.

Poly-zinc finger peptides of this invention may contain 2, 3, 4, 5, 6 or more zinc finger modules. Natural zinc finger modules of this invention may preferably be linked by canonical, flexible or structured linkers, as set out below and in WO 01/53480, the disclosure of which is hereby incorporated by reference. More preferably, the linkers are canonical linkers such as -TGEKP— (SEQ ID NO:3).

The poly-zinc finger peptides of this invention can be given useful biological functions by the addition of effector domains, creating chimeric zinc finger peptides. Preferably, such chimeric zinc finger peptides may be used to up- or down-regulate desired genes, in vitro or in vivo. Preferable effector domains include transcriptional repressor domains, transcriptional activator domains, transcriptional insulator domains, chromatin remodelling domains, enzymatic domains, and signalling/targeting sequences or domains. To cause a desired biological effect composite binding polypeptides can bind to one or more suitable nucleotide sequences in vivo or in vitro. Preferred DNA regions from which to effect the up- or down-regulation of specific genes include promoters, enhancers or locus control regions (LCRs). Other suitable regions within genomes, which may provide useful targets for composite binding polypeptides include telomeres and centromeres.

The expression of many genes is also achieved by controlling the fate of the associated RNA transcript. RNA molecules often contain sites for RNA-binding proteins, which determine RNA half-life. Hence, composite binding polypeptides can also control endogenous gene expression by specifically targeting RNA transcripts to either increase or decrease their half-life within a cell.

Composite binding polypeptides can also be fused to epitope tags, which can be detected by antibodies, and may therefore be used to signal the presence or location of a particular nucleotide sequence in a mixed pool of nucleic acids, or immobilised on the surface of a chip or other such surface.

Intracellular localization of composite binding polypeptides can be regulated, for example, by fusion to a localization domain, for example, a nuclear localization sequence or a localization domain as disclosed, for example, in PCT/US01/42377.

a. Nucleic Acid Binding Polypeptides

This invention preferably relates to nucleic acid binding polypeptides. Preferably, the binding polypeptides of the invention are DNA binding polypeptides. Particularly preferred examples of nucleic acid binding polypeptides are zinc finger peptides.

Zinc finger peptides typically contain strings of small nucleic acid binding domains, each stabilised by the co-ordination of zinc. These individual domains are also referred to as “fingers” and “modules”. A zinc finger recognises and binds to a nucleic acid triplet, or an overlapping quadruplet, in a DNA target sequence. However, zinc fingers are also known to bind RNA and proteins. Clemens, K. R. et al., (1993) Science 260: 530-533; Bogenhagen, D. F. (1993) Mol. Cell. Biol. 13: 5149-5158; Searles, M. A. et al., J. Mol. Biol. 301: 47-60 (2000); Mackay, J. P. & Crossley, M. (1998) Trends Biochem. Sci. 23: 1-4.

Preferably, there are 2 or more zinc fingers, for example 2, 3, 4, 5, 6, or 7 zinc fingers, in each zinc finger polypeptide. Advantageously, there are 3 or more zinc fingers in each zinc finger polypeptide.

All of the DNA binding residue positions of zinc finger peptides, as referred to herein, are numbered from the first residue in the α-helix of the finger, ranging from +1 to +9. “−1” refers to the residue in the framework structure immediately preceding the α-helix in a zinc finger peptide. Residues referred to as “++” are residues present in an adjacent (C-terminal) peptide. Where there is no C-terminal adjacent peptide, “++” interactions do not operate.

The α-helix of a zinc finger peptide aligns antiparallel to the target nucleic acid strand, such that the primary nucleic acid sequence is arranged 3′ to 5′ in order to correspond with the N-terminal to C-terminal sequence of the zinc finger peptide. Since nucleic acid sequences are conventionally written 5′ to 3′, and amino acid sequences N-terminus to C-terminus, the result is that when a target nucleic acid sequence and a zinc finger peptide are aligned according to convention, the primary interaction of the zinc finger peptide is with the “minus” strand of the nucleic acid sequence, since it is this strand which is aligned 3′ to 5′. These conventions are followed in the nomenclature used herein. It should be noted, however, that in nature certain zinc finger modules, such as zinc finger 4 of the protein GLI, bind to the “plus” strand of the nucleic acid sequence. See Suzuki et al. (1994) Nucl. Acids Rev. 22: 3397-3405; and Pavletich & Pabo, (1993) Science 261: 1701-1707. The present invention encompasses incorporation of such zinc finger peptides into DNA binding molecules.

Natural Zinc Finger Peptides.

In certain embodiments, this invention relates to natural zinc finger modules. As used herein, the term ‘natural’ with reference to a zinc finger, means that the DNA sequence which encodes a particular zinc finger, whether normally expressed in vivo or not, is found in nature, i.e. is part of the genome of a cell. A natural human zinc finger is one which is endogenous to the human genome, a natural mouse zinc finger is found in the mouse genome, and a natural viral zinc finger is found in a viral genome, etc. Natural zinc finger genes which have become integrated into the genome of a heterologous species by natural means, e.g., integration of a viral genome into a host genome, are considered to be endogenous to the host species within the context of this disclosure. A zinc finger module constructed or produced in vitro or extracted from an in vivo source is considered to be natural if its amino acid sequence matches that of the amino acid sequence encoded by its natural gene. The DNA sequence of the natural gene is not the defining aspect. Thus, polynucleotides encoding natural zinc finger modules may have a different sequence from that of the naturally-occurring sequence encoding the module, e.g., to adjust codon usage to optimise expression of the module in a particular expression system.

Preferably, sequences of zinc fingers used in the present invention are not mutated from their natural form. Advantageously, the natural zinc finger polypeptides are expressed in nature.

A natural zinc finger binding motif is a structure well known to those in the art and defined in, for example, Miller et al., (1985) EMBO J. 4: 1609-1614; Berg (1988) Proc. Natl. Acad. Sci. USA 85: 99-102; Lee et al., (1989) Science 245: 635-637; see also International patent applications WO 96/06166 and WO 96/32475, incorporated herein by reference.

In general, a natural zinc finger framework has the structure:

| | |
|---|---|
| SEQ ID NO: 12 | X₀₋₂ C X₁₋₅ C X₉₋₁₄ H X₃₋₆ ^{H}/_{C} |

where X is any amino acid, and the numbers in subscript indicate the possible numbers of residues represented by X (Formula A).

In a preferred aspect of the present invention, natural zinc finger nucleic acid binding motifs may be represented as motifs having the following primary structure:

| X₀₋₂ C X₁₋₅ C X₂₋₇ | (SEQ ID NO: 13) |
|---|---|
| X X X X X X X H X₃₋₆ ^{H}/_{C} | (SEQ ID NO: 14) |
| −1 1 2 3 4 5 6 7 | |

where X is any amino acid, and the numbers in subscript indicate the possible numbers of residues represented by X (Formula A′). The numbers −1 through 7 refer to amino acid position with respect to the beginning of the alpha-helical region of the zinc finger.

The Cys and His residues, which together co-ordinate the zinc metal atom, are marked in bold text and are usually invariant. However, all naturally-occurring zinc finger modules, even if they diverge from the above formula, are encompassed within the scope of this invention.

Zinc finger modules of formula A′ are often arranged in tandem within a natural zinc finger polypeptide, such that a zinc finger containing protein may have 2, 3, 4, 5, 6, 7, 8, 9 or more individual zinc finger motifs. In such a protein, individual zinc fingers are joined to each other by a polypeptide sequence known as a linker. Generally, such a natural linker lacks secondary structure, although the amino acids within the linker may form local interactions when the protein is bound to its target site. By ‘linker sequence’ is meant an amino acid sequence that links together adjacent zinc finger modules. For example, in a natural zinc finger protein, the linker sequence is the amino acid sequence which lies between the last residue of the α-helix in a zinc finger and the first residue of the β-sheet in the next zinc finger. The linker sequence therefore joins together two zinc fingers. For the purposes of the present invention, the last amino acid of the α-helix in a zinc finger is considered to be the final zinc coordinating histidine (or cysteine) residue, while the first amino acid of the following finger is generally a tyrosine/phenylalanine or another hydrophobic residue. Since some natural zinc fingers do not start with a hydrophobic residue (see Appendices), the start of a finger is sometimes harder to define from amino acid sequence (or indeed zinc finger structure), and so some flexibility must be allowed in this definition. Accordingly, in a natural zinc finger protein, threonine is often considered to be the first residue in the linker, and proline is the last residue of the linker. Thus, for example, in the natural Zif268 peptide the linker sequence is -TG(E/Q)(K/R)P— (SEQ ID NO: 15). Although natural linkers can vary greatly in terms of amino acid sequence and length, on the basis of sequence homology, the canonical natural linker sequence is considered to be -TGEKP— (SEQ ID NO:3). Hence, the preferred linker sequence to join zinc finger modules of the present invention is -TGEKP-.

Additionally, a ‘leader’ peptide may be added to the N-terminal zinc finger of a poly-zinc finger peptide to aid its expression, without changing the sequence of the natural zinc finger module. Preferably, the leader peptide is MAEERP (SEQ ID NO: 16) or MAERP (SEQ ID NO: 17).

In general, naturally occurring zinc finger modules may be selected from those proteins for which the DNA binding specificity is already known. For example, these may be the proteins for which a crystal structure has been resolved: namely Zif268 (Elrod-Erickson et al. (1996) Structure 4: 1171-1180), GLI (Pavletich & Pabo (1993) Science 261: 1701-1707), Tramtrack (Fairall et al. (1993) Nature 366: 483-487) and YY1 (Houbaviy et al. (1996) Proc. Natl. Acad. Sci. USA 93: 13577-13582). Furthermore, the sequence specificity of many naturally-occurring zinc fingers and zinc finger proteins are known. In addition, this invention further provides for the determination of the binding specificity of natural zinc finger modules for use in the present invention. See “Prediction of Binding Specificity,” infra.

Poly-Zinc Finger Peptides.

It is desirable that a ‘designer’ transcription factor for uses such as gene therapy and in transgenic organisms should have the ability to target virtually unique sites within any genome. For complex genomes such as in humans, an address of at least 16 bps is required to specify a potentially unique DNA sequence. Shorter DNA sequences have a significant probability of appearing several times in a genome, raising the possibility of obtaining undesirable non-specific gene targeting with a designed transcription factor targeted to such a shorter sequence. As individual zinc fingers only bind 3 to 4 nucleotides, it is therefore necessary to construct multi-finger polypeptides to target these longer sequences. A six-zinc finger peptide (with an 18 bp recognition sequence) could, in theory, be used for the specific recognition of a single target site and hence, the specific regulation of a single gene within any genome. In addition, a significant increase in binding affinity might also be expected, compared to a protein with fewer fingers. In simple terms, if a three-finger peptide (with a 9 bp recognition sequence) binds DNA with nanomolar affinity, two tandemly linked three-finger peptides might be expected to bind an 18 bp sequence with an affinity of 10⁻¹⁵-10⁻¹⁸ M. However, most previous attempts at producing high-affinity 6-finger peptides (poly-zinc finger peptides) based on fusions of two 3-finger domains have been unsuccessful in generating much of an improvement in affinity over 3-finger peptides. Liu, Q., Segal, D. J., Ghiara, J. B. & Barbas, C. F. III (1997) Proc. Natl. Acad. Sci. USA 94: 5525-5530; Kim, J-S. & Pabo, C. O. (1998) Proc. Natl. Acad. Sci. USA 95: 2812-2817; Kamiuchi, T., Abe, E., Imanishi, M., Kaji, T., Nagaoka, M. & Sugiura, Y. (1998) Biochemistry 37: 13827-13834. To optimise both the affinity and specificity of 6-finger peptides, a fusion of three 2-finger domains has been shown to be advantageous. Moore, M., Klug, A. & Choo, Y. (2001) Proc. Natl. Acad. Sci. USA 98: 1437-1441; and WO 01/53480. Therefore, in one embodiment, 2-finger units are linked to make poly-zinc finger nucleotide-binding domains. A pool of 4096 such 2-finger units, that recognise all possible 6 bp sequences (4⁶=4096), represents an archive sufficient to rapidly create universal nucleic acid recognition, by simple linkage, in an “off-the-shelf” manner. See Moore et al., supra and WO 01/53480.

Poly-zinc finger peptides according to this invention may be constructed containing 2, 3, 4, 5, 6 or more zinc finger modules. Such poly-zinc finger peptides may contain inter-finger linkers other than the canonical (TGEKP) linker sequence, as described, for example, in WO 01/53479; Moore, M., Choo, Y. & Klug, A. (2001) Proc. Natl. Acad. Sci. USA 98: 1432-1436; and Moore, M., Klug, A. & Choo, Y. (2001) Proc. Natl. Acad. Sci. USA 98: 1437-1441. Briefly, linker sequences may be flexible or structured but, in general, will not form base-specific interactions with the target nucleotide sequence. A ‘flexible’ linker is defined as one which does not form a specific secondary structure in solution, whereas a ‘structured’ linker is defined as one that adopts a particular secondary structure in solution. Preferably, flexible linkers include the sequences GGERP (SEQ ID NO:18), GSERP (SEQ ID NO:19), GGGGSERP (SEQ ID NO:20), GGGGSGGSERP (SEQ ID NO:21), GGGGSGGSGGSERP (SEQ ID NO:22), GGGGSGGSGGSGGSGGSERP (SEQ ID NO:23). Preferably, the structured linker comprises an amino acid sequence that is not capable of specifically binding nucleic acid. More preferably, the structured linker comprises the amino acid sequence of TFIIIA finger IV. Alternatively, or in addition, the structured linker is derived from a zinc finger by mutation of one or more of its base contacting residues to reduce or abolish nucleic acid binding activity of the zinc finger. The zinc finger may be finger 2 of wild type Zif268 mutated at positions −1, 2, 3 and/or 6.

In one embodiment, this invention provides for the construction and screening of poly-zinc finger peptides containing at least one natural zinc finger module.

In another embodiment, this invention provides for the construction and screening of poly-zinc finger peptides containing at least one natural zinc finger module, linked with the canonical linker sequence -TGEKP— (SEQ ID NO:3).

In one embodiment, methods for the construction and use of poly-zinc finger peptide comprising natural zinc finger modules are provided.

In another embodiment, methods for the construction and use of poly-zinc finger peptide comprising natural zinc finger modules, linked with the canonical linker sequence -TGEKP— (SEQ ID NO:3), are provided.

In a further embodiment, methods for the construction and use of poly-zinc finger peptides comprising at least one natural zinc finger module, containing either flexible or structured linkers (as described above and in WO 01/53480), are provided.

b. Advantages of Natural Zinc Finger Modules

Zinc finger modules are compact and stable structures of approximately 30 amino acids, which contain the fill information required to bind a nucleic acid triplet or overlapping quadruplet. As such, they have proven to be extremely versatile scaffolds for engineering novel DNA-binding domains. See, for example, Rebar, E. J. & Pabo, C. O. (1994) Science 263, 671-673; Jamieson, A. C., Kim, S.-H. & Wells, J. A. (1994) Biochemistry 33, 5689-5695; Choo, Y. & Klug, A. (1994) Proc. Natl. Acad. Sci. U.S.A. 91, 11163-11167; Choo, Y., Sanchez-Garcia, I. & Klug, A. (1994) Nature 372, 642-645; Wu, H., Yang, W.-P. & Barbas III, C. F. (1995) Proc. Natl. Acad. Sci. USA 92, 344-348; Greisman, H. A. & Pabo, C. O. (1997) Science 275, 657-661; Isalan, M., Klug, A. & Choo, Y. (1998) Biochemistry 37, 12026-12033; Choo, Y. (1998) Nature Struct. Biol. 5, 264-265; Segal, D. J., Dreier, B., Beerli, R. R. & Barbas, C. F. (1999) Proc. Natl. Acad. Sci. USA 96, 2758-2763; Isalan, M. & Choo, Y. (2000) J Mol Biol 295, 471-477; and Beerli, R. R., Dreier, B., Barbas, C. F. (2000) Proc Natl Acad Sci USA 97, 1495-500. The resulting engineered zinc finger domains have increased our knowledge of sequence-specific DNA recognition, as well as provided a wide range of potential tools for medicine and biotechnology.

As a result of these and other studies on zinc finger engineering, it has been recognised that an individual zinc finger module does not necessarily recognise a simple nucleotide triplet, as was first thought; but instead, can bind to an overlapping quadruplet of double stranded DNA. See, for example, Isalan et al. (1997) Proc Natl Acad Sci USA 94, 5617-5621; and WO98/53057). In this respect, zinc finger engineering strategies have been particularly important for deciphering the mechanism and specificity of these interactions.

With the recent completion of the human genome project and the rapidly advancing fields of transgenic animals and plants, thousands of uncharacterised (and characterised) genes have (and will) become valid targets for functional genomics and other such projects. Concomitantly, engineered zinc finger peptides (often as a component of “designer” transcription factors) are emerging as one of the most universal and desirable ways of regulating the expression of specific genes within cells. See, for example, Choo, Y., Sanchez-Garcia, I. & Klug, A. (1994) Nature 372: 642-645; Beerli, R. R., Dreier, B. & Barbas, C. F. III (2000) Proc. Natl. Acad. Sci. USA 97: 1495-1500; Kim, J-S. & Pabo, C. O. (1998) Proc. Natl. Acad. Sci. USA 95: 2812-2817; Kang, J. S. & Kim, J-S. (2000) J. Biol. Chem. 275: 8742-8748; Zhang et al. (2000) J. Biol. Chem. 275:33,850-33,860; Liu et al. (2001) J. Biol. Chem. 276:11,323-11,334; Ren et al. (2002) Genes. Devel. 16:27-32; and WO 00/41566.

Notwithstanding the remarkable progress in zinc finger engineering, there remain several issues that limit the use of engineered zinc fingers for such applications. Points of particular concern include the potential immunogenicity of non-natural zinc fingers, and the ‘fine-tuning’ of particular aspects of the protein-DNA interactions to obtain optimal and specific zinc finger-nucleic acid contacts.

The present invention overcomes problems such as immunogenicity and optimal binding specificity, by exploiting the vast repertoire of naturally occurring zinc fingers to construct targeted zinc finger proteins having novel specificities.

Immunogenicity

The main function of the immune system is to detect, and render harmless, foreign particles which have invaded the body as a whole, or individual cells or organs. ‘Foreign’ in this context means non-host, i.e. a substance which has originated from a different species, or one which has originated as a result of a mutation al event (such as might generate a malignant cell). On encountering such an antigenic particle, either in solution or on the surface of an infected cell, the body's defences rapidly destroy/remove it by complex pathways which involve the interaction of many members of the immune system. For a good overview of immunology see Roitt, Essential Immunology, Blackwell Science Ltd. and Roitt, I., Brostoff, J. & Male, D. Immunology, 4^{th} Ed. Mosby. Hence, all biological therapeutic agents, such as peptides, nucleic acids, viruses, etc., risk eliciting an immune response in the recipient. Particularly for cases in which repeated doses of a therapeutic agent are required, this response can be strong and potentially dangerous to the host organism.

The immune system functions through either innate or adaptive responses. The innate response is usually the body's first internal line of defence. Phagocytic cells recognise and bind to foreign objects in extracellular environments. Once bound, the foreign object is internalised and destroyed. Foreign therapeutic agents such as peptides and nucleic acids, which are administered directly to the blood stream of the recipient, risk being detected and possibly destroyed before they even reach their intended target. This response is one of primitive non-specific recognition of non-host agents, and does not adapt with time or exposure to the antigen.

Foreign therapeutic agents (or infectious agents such as bacteria and viruses), which evade the innate immune response and may have been successfully delivered to a particular cell have not necessarily avoided the host's immune system. Proteins that are expressed in cells are routinely degraded within lysosomes, and short peptide fragments, generally of between 6 and 9 amino acids, are transported to the cell surface and presented to the host's immune system. This is the start of the host's second internal defence mechanism against invasion, the adaptive immune response. The proteins responsible for displaying such peptide fragments are known as major-histocompatibility complexes (MHC) proteins. Lymphocyte cells, known as T-lymphocytes, dock with the MHC proteins and scan the peptide fragments displayed. Contact of a T-lymphocyte with a fragment specifically recognised as not belonging to the host organism initiates an immunological cascade which ultimately results in the host cell being destroyed or undergoing apoptosis. This mechanism is one of specific recognition, and once recognised as foreign, the antigen is ‘remembered’ so that any future invasions by the agent are dealt with more and more rapidly. B-cells are another type of lymphocyte that recognise extracellular particles and then produce and release antibodies to help combat the agent.

To avoid potentially damaging the host organism and to ensure the successful delivery and action of a therapeutic peptide it is important to make it as much like a host protein as is reasonably possible. In the case of synthesised therapeutic antibodies for human use, a great deal of work has gone in to the ‘humanisation’ of antibodies produced by other animal species (See EP 0239400). In this invention we present a solution for the equivalent problem associated with zinc finger therapeutic peptides.

To some extent, prior art zinc finger engineering strategies have attempted to minimise the risk of eliciting immune responses by using an engineering scaffold that is compatible with (i.e. that originates from) the recipient, and by limiting the sizes of the varied regions within the final product. For example, typical engineered zinc fingers utilize a scaffold such as the three-finger DNA-binding domain of Zif268 (containing approximately 100 amino acid residues). Because the amino acid sequence of Zif268 is completely conserved in a variety of species, including mice and humans, the scaffold is not itself immunogenic in these species. However, in order to engineer new DNA-binding domains, stretches of approximately 7 amino acids must be varied within each zinc finger. These sequences of 7 amino acids represent modifications in positions −1, 1, 2, 3, 4, 5, and 6 of the α-helix of each finger. Although these engineered regions are considered to be relatively small, they are approximately the length of the peptide fragments displayed on the surface of cells by MHC molecules. Hence, they may provide antigenic peptide fragments in several registers of the amino acid sequence, which may result in dangerous and/or undesirable immune responses in the host.

Accordingly, it is not known whether this type of engineering strategy will be entirely sufficient to avoid all potential undesirable effects, or indeed whether it will create the most optimal framework for all zinc finger-nucleic acid interactions.

In addition to the zinc fingers themselves, it is also possible that inter-finger linker sequences could present potential immunological problems. Fortunately, natural zinc finger proteins display strong conservation and homology in their linker sequence. A very large number of natural fingers are joined by the canonical linker peptide -TGEKP- (SEQ ID NO:3), located between the final zinc chelating residue (usually histidine) of the first finger, and the first residue of the second finger (usually a large hydrophobic residue such as tyrosine or phenylalanine, which begins the β-sheet). Hence, the use of the canonical linker sequence -TGEKP— (SEQ ID NO:3), to join natural zinc finger modules in a non-natural order, will reduce the possibility of eliciting an undesirable immune reaction to a minimum. Furthermore, there are so many natural zinc fingers which are already joined by canonical linker sequences, that if deemed necessary, the database of natural zinc fingers used for the construction of poly-zinc finger peptides may be restricted to those already flanked by such linkers.

The periodicity of zinc fingers and their amenability to linkage using the TGEKP (SEQ ID NO:3) motif is illustrated in Table 2.

TABLE 2

A functional three-finger DNA-binding domain based on the
peptide sequence of Zif268. TGEKP linker motifs are underlined.
The helical residues of each zinc finger are numbered relative
to the first helical position, position +1. Conserved Cysteines
and Histidines forming the classical Cys₂His₂ zinc finger core
are shown in bold.

| | |
|---|---|
| α-HELIX | LINKER |

| | | | | |
|---|---|---|---|---|
| | | −1123456 | | |
| YA CPVESCDRRFS | (SEQ ID NO: 24) | RSDELTRHIRIH | (SEQ ID NO: 25) | TGEKP |
| FQ CRI CMRNFS | (SEQ ID NO: 26) | RSDHLSTHIRTH | (SEQ ID NO: 27) | TGEKP |
| FA CDI CGRKFA | (SEQ ID NO: 28) | RSDERKRHTKIH | (SEQ ID NO: 29) | TGEKP |

Fine-Tuning of Zinc Finger-Nucleic Acid Interactions.

It has previously been shown that zinc fingers cannot simply be regarded as independent nucleic acid-binding modules. Isalan, M., Klug, A. & Choo, Y. (1998) Biochemistry 37, 12026-12033; Isalan, M., Choo, Y. & Klug, A. (1997) Proc Natl Acad Sci 94,5617-5621. The interactions between adjacent zinc fingers can be complex and involve overlap of binding sites, which means that optimal interfaces are not easily engineered through rational design. Combinatorial library selection systems, which if designed correctly necessarily result in interface compatibility, can help to engineer better optimisation of the zinc finger-nucleic acid interface. See, for example, WO98/53057. However, all library selection systems suffer from the problem of library size, whereby because of physical constraints, it is impossible to include an exhaustive combination of randomisations to cover all potentially important sequence-space. For example, to optimise the zinc finger-nucleic acid interface, subtle amino acid variations may be needed, even from positions outside the recognition α-helix. Furthermore, alternative approaches to zinc finger engineering, such as ‘affinity maturation’ through random mutation or gene shuffling, which may (to a limited extent) increase the coverage of sequence space, may also raise the probability of generating undesirable immunological problems. Hence, it is possible that the creation of truly optimal zinc finger domains for recognition of specific nucleic acid sequences may be outside the scope of traditional engineering strategies.

In contrast, naturally occurring zinc finger modules have already been ‘fine-tuned’ by thousands of years of natural selection and are, under normal circumstances, non-immunogenic in their host organism. The human genome project has revealed that zinc finger-containing proteins constitute the second most abundant family of proteins in humans, with well over 600 members. Since zinc finger proteins usually contain several individual zinc finger modules, the human genome provides a repertoire of thousands of natural zinc finger modules for the creation of composite binding polypeptides. Furthermore, because there are only 64 (=4³) possible 3 bp sequences and 256 (=4⁴) possible 4 bp sequences, it is likely that a natural zinc finger domain exists which is capable of binding to every potential 3- or 4-nucleotide target sequence. Consequently, natural zinc fingers are a very useful resource for the production of composite binding polypeptides comprising zinc fingers. At present, the natural binding site of many natural zinc finger modules is not known. Thus, to be useful for the construction of composite binding polypeptides, nucleotide sequence preferences for certain natural zinc fingers are determined according to rules tables disclosed in the following section (“minding Specificity of Natural Zinc Finger Modules”).

To create optimal poly-zinc finger peptides the potentially significant problem of interface incompatibility must be addressed, since natural zinc finger modules will not necessarily be compatible with each other when juxtaposed. In this respect, a library construction and screening system is preferably employed which links natural zinc finger modules in non-natural combinations, and screens them against possible target sequences of greater than 3 or 4 bp in length (which represents the possible binding site of a single zinc finger module), to determine optimal 2- or 3-finger domains. In this way, the cooperative nature of zinc finger binding is taken into account in the design and selection of composite binding polypeptides, and in the determination of the sequence specificity of their binding. In one embodiment, a library of poly-zinc finger peptides containing at least one natural zinc finger module is provided. Preferably, poly-zinc finger peptides of the library contain at least two natural zinc finger modules.

c. Binding Specificity of Natural Zinc Finger Modules

Disclosed herein are certain improvements to current limitations on the use of customised zinc finger nucleic acid binding domains, through the use of natural zinc finger modules. By using either natural 1-finger or 2-finger sub-domains, and/or novel combinatorially-mixed, pre-selected 2-finger sub-domains, it is possible to construct poly-zinc finger peptides that bind any desired nucleotide target sequence, using non-natural combinations of natural zinc fingers.

This approach is particularly suited for human gene therapy applications, but the invention is not just limited to zinc finger modules encoded by the human genome. For applications within transgenic animals such as mice, chicken, etc., the same system can be used, but incorporating natural zinc finger modules from those species instead (see Example 3). The genome of any organism (e.g., animal, plant, bacterium, virus, etc.) can thus provide a genetic ‘toolbox’ of non-immunogenic, structurally optimised zinc fingers for applications in that organism.

Before such zinc finger modules can be utilised, however, it is essential that their optimal binding site is determined, in isolation, or preferably as part of a 2- or 3-finger subdomain. Natural zinc finger modules are advantageously fused into subdomains comprising two or three zinc finger modules in random arrangement, optionally comprising an anchor finger, then subjected to binding site analysis. An ‘anchor’ zinc finger is one for which the binding specificity is known, such as, for example, finger 1 or finger 3 of Zif268, each of which binds the sequence 5′-GCG-3′. An anchor finger is attached to the N— or C-terminus of the zinc finger module(s) or subdomain for which the binding specificity is to be determined, and acts as an anchor to set the binding register for the binding site selection. For example, if the binding site preference of a pair of natural zinc fingers is to be determined, finger 1 of Zif268 may be fused to the N-terminus of the pair of natural fingers, and a 5′-GCG-3′ anchor sequence is placed at the 3′ end of 6 or more randomised nucleotides. Selection of the optimal binding site may thus be conducted with an oligonucleotide containing the sequence 5′-XXX-XXX-GCG-3′ (SEQ ID NO:30), where X is any specified nucleotide. The anchor sequence thereby allows the binding site preference of the zinc finger libraries to be easily determined. Such procedures are described in the Examples.

Screening for Zinc Finger Binding Specificity

There are various approaches, known to those in the art, for screening nucleic acid binding peptides for their binding specificity. To determine the binding specificity of, for example, zinc finger peptides, procedures can be conducted using: (a) a library of zinc fingers and a specified target sequence—to select one or more zinc finger peptides with a particular binding preference; or (b) a single zinc finger peptide and a random population of target sequences—to select one or more optimal binding sites for a particular peptide. For many applications, such as for the creation of transcription factors for regulating specific gene activity, it is often preferable to screen zinc finger libraries against specific target sequences. In this way, the search is geared towards a particular application. However, if the function or binding specificity of a natural protein is the object of the investigation, a library of potential binding sites can be screened using a single peptide. Some such methods are outlined below.

A typical method for screening libraries of nucleic acid binding polypeptides against specific target sites is that of phage display. Phage display protocols generally involve expressing the peptides under study as fusions with the gIII major coat protein of bacteriophage (J. McCafferty, R. H. Jackson, D. J. Chiswell, (1991) Protein Engineering 4, 955-961). Suitable protocols for the selection of zinc finger peptides have been described and are well known to those in the art. See, for example, Choo, Y. & Klug, A. (1994) Proc. Natl. Acad. Sci. U.S.A. 91, 11163-11167; Choo, Y., Sanchez-Garcia, I. & Klug, A. (1994) Nature 372, 642-645; Choo, Y. (1998) Nature Struct. Biol. 5, 264-265; Choo, Y. & Klug, A. (1997) Curr. Opin. Str. Biol. 7, 117-125; 7 Isalan, M., Klug, A. & Choo, Y. (1998) Biochemistry 37, 12026-12033; Isalan, M. & Choo, Y. (2000) J Mol Biol 295, 471-477; Isalan, M., Choo, Y. & Klug, A. (1997) Proc Natl Acad Sci 94, 5617-5621; WO 01/53480, WO 01/53479, WO 96/06166, WO 98/53057, WO 98/53058, WO 98/53059 and WO 98/53060 and references cited therein; see also Examples, ii-fi-a. In general, sequences comprising target sites are bound, such as through biotin-streptavidin, to a solid support, such as a magnetic particle, or the surface of a tube or well. A solution of phage expressing members of a library of zinc finger peptides is then added to the immobilised target site. Non-bound phage are washed away and bound phage (containing the DNA encoding the bound zinc finger peptide), are collected. The collected phage sample is usually reused in further rounds of selection to enrich for the tightest binding zinc finger peptide.

Phage display protocols based on random mutagenesis of zinc finger modules are known to have a number of limitations. First, as discussed above, the library size that can be expressed on the surface of phage is limited by the efficiency of procedures such as cloning and transformation. Furthermore, the efficiency of incorporation of gIII-zinc finger fusions into phage and hence, zinc finger peptide expression, is determined by the number of zinc finger modules. Therefore, 2-finger peptides are expressed more efficiently than 3-finger peptides and so on. For this reason, phage display protocols are generally limited to the assay of polypeptides comprising 3 or fewer zinc finger modules.

An alternative to phage display is an in vitro selection system. In such a system, libraries of zinc fingers can be produced by PCR using degenerate primer oligonucleotides. Target binding sites are added to the end of the DNA encoding the zinc finger peptide. Zinc finger peptide expression may be performed directly from PCR products using an in vitro expression kit, such as the TNT T7 Quick Coupled Transcription/Translation System for PCR DNA (Promega, Madison, Wis., USA), or another suitable expression system. The components of the expression reaction (including the zinc finger gene/binding site) are compartmentalised by suspension in an emulsion, in such a way that (on average) only one copy of the zinc finger gene/binding site is present in each compartment. See, for example, Tawfik, D. S. & Griffiths, A. D. (1998) Nat. Biotechnol. 16: 652-656. Zinc finger peptides which bind the specified target site (and the gene encoding them) can be collected using, for example, a suitable epitope tag (such as myc, FLAG or HA tags), and the non-bound binding sites/zinc finger genes are removed. The genes encoding zinc finger peptides that bind the required target site can then be amplified by PCR and used in further rounds of selection if required.

A preferred method for selecting a zinc finger peptide which binds a specified target sequence is described in Example 4. Briefly, the DNA encoding a library of zinc finger peptides with an attached epitope tag is diluted into as many aliquots as it is possible to screen (e.g. 384 or 1534 aliquots). This creates pools of sub-libraries with reduced numbers of variants. The DNA is then amplified by PCR and used to produce protein, from a suitable in vitro expression system, as described above. A specified binding site with an attached biotin molecule, and a horse radish peroxidase (HRP)-conjugated antibody to the peptide-attached epitope tag may then be added. Binding site/bound zinc finger/antibody complexes may be collected by binding to streptavidin and the samples are washed to remove unbound zinc finger and antibodies. The samples containing the highest amount of bound zinc finger peptide can be detected by adding an HRP substrate solution. The original DNA stock from such positive samples may then be diluted into aliquots (as above), PCR-amplified and used for the next round of selection. In this way, pools of zinc finger encoding genes with the desired activity are isolated, subdivided into pools of reduced variation and re-isolated until the most active clone is identified.

Principal advantages of the in vitro systems described above are: (a) there is virtually no limit to the library size which can be screened (up to 10¹² different PCR products can easily be made); and (b) polypeptides comprising larger numbers of linked zinc finger modules (e.g., 4, 5, 6, 7, or more) can be assayed. Another in vitro selection system which can be used is polysome/ribosome display. See, for example, Mattheakis, L. C., Bhatt, R. R. & Dower, W. J. (1994) Proc. Natl. Acad. Sci. USA. 91: 9022-9026; and WO 00/27878.

Protocols for the reverse selection procedure, i.e. the selection of a particular binding site from a mixed population using a single nucleic acid binding polypeptide, include SELEX (systematic evolution of ligands by exponential enrichment) and microarray techniques.

The SELEX procedure has been well described. See, for example, Drolet, D. W., Jenison, R. D., Smith, D. E., Pratt, D. & Hicke, B. J. (1999) Comb. Chem. High Throughput Screen 2: 271-278; Burden, D. A. & Osheroff, N. (1999) J. Biol. Chem. 274: 5227-5235; Shultzaberger, R. K. & Schneider, T. D. (1999) Nucleic Acids Res. 27: 882-887; Marozzi, A., Meneveri, R., Giacca, M., Gutierrez, M. I., Siccardi, A. G. & Ginelli, E. (1998) J. Biotechnol. 15: 117-128; and U.S. Pat. Nos. 5,270,163; 5,475,096; 5,595,877; 5,670,637; 5,696,249; 5,817,785 and 6,331,398. A single nucleic acid binding polypeptide is expressed, either in vitro or in vivo, and screened against a library of target sequences. Nucleic acid binding polypeptides are collected (along with any bound target sites) using an epitope tag (as above) or another suitable procedure. Bound target sites are amplified by PCR and may be used in further rounds of selection, to enrich for the optimal binding site, or sequenced.

Microarray technology provides a method of screening a particular polypeptide or nucleic acid against thousands to millions of target sequences on a single slid support such as, for example, a glass or nitrocellulose slide. For example, the members of a library encoding polypeptides comprising 2 linked zinc fingers will bind a 6 bp recognition sequence. Hence, there are 4096 (=4⁶) unique binding sites for such a library. All 4096 of these sites can be arrayed onto a single glass slide, for example, allowing a specified 2-finger peptide to be screened simultaneously against every possible binding site. The amount of binding to each target sequence can be visualised and quantified using simple fluorescence measurements. For example, the zinc finger peptide may be expressed in vitro, or on the surface of phage. Isolated zinc finger peptides may contain an epitope tag for labelling purposes, whereas bound phage can be detected using a primary antibody against a phage coat protein, such as gVIII. A secondary antibody conjugated to, for example, R-phycoerythrin, horseradish peroxidase or alkaline phosphatase, can be used to provide a visible, quantifiable signal when a suitable substrate is applied. See, for example, Bulyk et al. (2001) Proc. Natl. Acad. Sci. USA:98,:13, 7158-7163, which is incorporated, by reference, in its entirety.

Prediction of Binding Specificity

The screening approaches described above rely on the assay of large libraries of randomly-selected natural zinc finger modules, to obtain one or more zinc finger modules that optimally bind a particular target nucleic acid sequence. In order to simplify the process further and ensure a more rapid selection of optimal zinc finger modules for a particular target site, sub-libraries can be created. In-this disclosure, the term ‘sub-library’ refers to a library of natural zinc finger modules that have been roughly categorised according to their predicted binding specificity. For example, the total population of natural zinc fingers can be sub-divided to create libraries comprising zinc finger modules whose predicted binding sites are guanine (G) rich, cytosine (C) rich, adenine (A) rich or thymine (T) rich. Alternatively, sub-libraries can be categorised as binding G in the 3′ position, in the central position, or in the 5′ position of a nucleotide triplet, etc. Alternatively, sub-libraries can be created which comprise zinc finger modules predicted to bind a particular triplet sequence such as, for example, GGG, GGA, GGC, GGT, GAG, GCG, GTG, etc. This approach combines knowledge of the modes of zinc finger-nucleic acid recognition, gained from studies on artificial zinc finger variants, with the benefits of combinatorial library selection. It also takes into account the fact that concerted interactions between adjacent zinc fingers, i.e. overlapping contacts, can affect the binding affinity and/or specificity of individual zinc fingers. See, for example, Isalan, M., Klug, A. & Choo, Y. (1998) Biochemistry 37, 12026-12033; Isalan, M., Choo, Y. & Klug, A. (1997) Proc Natl Acad Sci 94,5617-5621. Thus, for example, a composite binding polypeptide comprising two fingers, each having a predicted binding specificity for a particular triplet, can be easily screened to determine if that pair of fingers are compatible with each other for binding to the 6-nucleotide target site comprising their individual target sequences. This strategy is described further in the Examples.

For the process of creating sub-libraries of natural zinc fingers according to predicted binding preference, the rules set forth in international patent applications WO 96/06166, WO 98/53057, WO 98/53058, WO 98/53059 and WO 98/53060, and described in more detail below, are used. These rules allow the assignment of an amino acid residue, in an appropriate position of the recognition region of a zinc finger module (generally comprising amino acids −1 through +6, with respect to the start of the alpha-helical portion of the finger), which will bind a specified nucleotide in a triplet or quadruplet target subsite. However, these rules can also be used to predict the sequence of a target subsite that would be preferentially bound by a zinc finger of given amino acid sequence. In particular, the identity of the amino acid residing at a particular position in the recognition region of a natural zinc finger module can be used to predict the identity of a nucleotide at a particular location in a target subsite. These ‘rules’ should be considered as a guide to target site preference and not a guaranteed prediction, as binding site specificity may be determined by variations elsewhere in the zinc finger module (i.e. outside of the recognition region), may be influenced by context, or may be influenced by factors as yet unknown. It should also be noted that some rules may be more generally applicable than others.

In the application of these rules, it should be noted that the recognition region of a zinc finger aligns such that the N-terminal to C-terminal sequence of the finger is arranged along the nucleic acid strand to which it binds in a 3′-to-5′ direction. As a result, when a zinc finger sequence and a nucleic acid sequence (to which the finger binds) are aligned, the primary interactions occur between the zinc finger and the ‘minus’ strand of the nucleic acid sequence (i.e. the strand which has a 3′-to-5′ orientation). Furthermore, as stated above, the recognition region of a zinc finger comprises amino acids −1 through +6, with respect to the start of the alpha-helical portion of the finger. With respect to a particular zinc finger, an amino acid residue designated ++2 refers to the residue present in the adjacent (in the C-terminal direction) zinc finger, which (in certain instances) buttresses an amino acid-nucleotide interaction and/or participates in a cross-strand interaction with a nucleotide.

Thus, the following set of rules can be used to predict a 3 bp target subsite for a given natural zinc finger module: (a) if the 5′ base in the triplet is G, then position +6 in the α-helix is Arg; or position +6 is Ser or Thr and position ++2 is Asp; (b) if the 5′ base in the triplet is A, then position +6 in the α-helix is Gln and ++2 is not Asp; (c) if the 5′ base in the triplet is T, then position +6 in the α-helix is Ser or Thr and position ++2 is Asp; (d) if the 5′ base in the triplet is C, then position +6 in the α-helix may be any amino acid, provided that position ++2 in the α-helix is not Asp; (e) if the central base in the triplet is G, then position +3 in the α-helix is His; (f) if the central base in the triplet is A, then position +3 in the α-helix is Asn; (g) if the central base in the triplet is T, then position +3 in the α-helix is Ala, Ser or Val; provided that if it is Ala, then one of the residues at −1 or +6 is a small residue; (h) if the central base in the triplet is C, then position +3 in the α-helix is Ser, Asp, Glu, Leu, Thr or Val; (i) if the 3′ base in the triplet is G, then position −1 in the α-helix is Arg; (j) if the 3′ base in the triplet is A, then position −1 in the α-helix is Gln; (k) if the 3′ base in the triplet is T, then position −1 in the α-helix is Asn or Gln; (l) if the 3′ base in the triplet is C, then position −1 in the α-helix is Asp.

Furthermore, a natural zinc finger module may be capable of binding specifically to a four-nucleotide target subsite that overlaps with the target subsite of an adjacent zinc finger. In this case a different set of ‘rules’ can be used to determine predicted binding sites for each zinc finger module. Accordingly, in the description below, the overlapping 4 bp binding site is described such that position 4 is the 5′ base of a typical triplet binding site, position 3 is the central position of a typical triplet, position 2 is the 3′ position of a typical triplet, and position 1 is the complement of the nucleotide which is contacted by the cross strand interaction from the +2 position of the zinc finger module. Position 1 can also be considered to be the 5′ base of the triplet or quadruplet contacted by an adjacent (in the N-terminal direction) finger, if present.

Binding to each base of a quadruplet by an α-helical zinc finger nucleic acid binding motif in a natural protein can be predicted with reference to the following rules: (a) if base 4 in the quadruplet is G, then position +6 in the α-helix is Arg or Lys; (b) if base 4 in the quadruplet is A, then position +6 in the α-helix is Glu, Asn or Val; (c) if base 4 in the quadruplet is T, then position +6 in the α-helix is Ser, Thr, Val or Lys; (d) if base 4 in the quadruplet is C, then position +6 in the α-helix is Ser, Thr, Val, Ala, Glu or Asn; (e) if base 3 in the quadruplet is G, then position +3 in the α-helix is His; (f) if base 3 in the quadruplet is A, then position +3 in the α-helix is Asn; (g) if base 3 in the quadruplet is T, then position +3 in the α-helix is Ala, Ser or Val; provided that if it is Ala, then one of the residues at −1 or +6 is a small residue; (h) if base 3 in the quadruplet is C, then position +3 in the α-helix is Ser, Asp, Glu, Leu, Thr or Val; (i) if base 2 in the quadruplet is G, then position −1 in the α-helix is Arg; 6) if base 2 in the quadruplet is A, then position −1 in the α-helix is Gln; (k) if base 2 in the quadruplet is T, then position −1 in the α-helix is His or Thr; (l) if base 2 in the quadruplet is C, then position −1 in the α-helix is Asp or His; (m) if base 1 in the quadruplet is G, then position +2 is Glu; (n) if base 1 in the quadruplet is A, then position +2 Arg or Gln; (o) if base 1 in the quadruplet is C, then position +2 is Asn, Gln, Arg, His or Lys; (p) if base 1 in the quadruplet is T, then position +2 is Ser or Thr.

The above rules may be further refined to those described below: (a) if base 4 in the quadruplet is G, then position +6 in the α-helix is Arg; or position +6 is Ser or Thr and position ++2 is Asp; (b) if base 4 in the quadruplet is A, then position +6 in the α-helix is Gln and ++2 is not Asp; (c) if base 4 in the quadruplet is T, then position +6 in the α-helix is Ser or Thr and position ++2 is Asp; (d) if base 4 in the quadruplet is C, then position +6 in the α-helix may be any amino acid, provided that position ++2 in the α-helix is not Asp; (e) if base 3 in the quadruplet is G, then position +3 in the α-helix is His; (f) if base 3 in the quadruplet is A, then position +3 in the α-helix is Asn; (g) if base 3 in the quadruplet is T, then position +3 in the α-helix is Ala, Ser or Val; provided that if it is Ala, then one of the residues at −1 or +6 is a small residue; (h) if base 3 in the quadruplet is C, then position +3 in the α-helix is Ser, Asp, Glu, Leu, Thr or Val; (i) if base 2 in the quadruplet is G, then position −1 in the α-helix is Arg; (j) if base 2 in the quadruplet is A, then position −1 in the α-helix is Gln; (k) if base 2 in the quadruplet is T, then position −1 in the α-helix is Asn or Gln; (l) if base 2 in the quadruplet is C, then position −1 in the α-helix is Asp; (m) if base 1 in the quadruplet is G, then position +2 is Asp; (n) if base 1 in the quadruplet is A, then position +2 is not Asp; (o) if base 1 in the quadruplet is C, then position +2 is not Asp; (p) if base 1 in the quadruplet is T, then position +2 is Ser or Thr.

The rules therefore predict that the presence of an Asp (D) residue at position +2 will preclude binding to either A or C by an amino acid at position +6 in an adjacent N-terminal finger. Isalan, M., Klug, A. & Choo, Y. (1998) Biochemistry 37, 12026-12033; Isalan, M., Choo, Y. & Klug, A. (1997) Proc Natl Acad Sci 94, 5617-56212. Therefore, natural zinc fingers containing Asp, Glu, Asn or Gln at +6 are likely to be incompatible with any C-terminal finger containing an Asp residue at position +2. Although there are many such rules to describe the overlap between adjacent zinc fingers, a certain degree of degeneracy exists in these rules. Nonetheless, physical selection procedures (e.g., library construction and screening) can be used to extract optimal pairs of fingers for any given target subsite interface.

Not all natural zinc fingers have a DNA-binding function. For example, it is known that many zinc fingers, such as those from TFIIIA, bind to RNA (Clemens, K. R. et al., (1993) Science 260: 530-533; Bogenhagen, D. F. (1993) Mol. Cell. Biol. 13: 5149-5158; Searles, M. A. et al., J. Mol. Biol. 301: 47-60 (2000)). The rules governing RNA binding by zinc fingers are less well understood than those of DNA binding, but some RNA binding zinc fingers can be identified on the basis of a characteristic sequence motif. Clemens, K. R. et al., (1993) Science 260: 530-533; Bogenhagen, D. F. (1993) Mol. Cell. Biol. 13: 5149-5158; Searles, M. A. et al. (2000) J. Mol. Biol. 301: 47-60. Furthermore, some zinc fingers, such as those from the protein Ikaros, are able to form protein-protein interactions. Such zinc fingers often contain large hydrophobic patches. Mackay, J. P. & Crossley, M. (1998) Trends Biochem. Sci. 23: 1-4.

To this end, applied bioinformatic processing can help to determine which candidates in a particular genome are best suited to fulfilling a particular function, such as DNA-binding. In the case of zinc fingers, numerous documented databases exist denoting amino acid residues that are most likely to be found at particular positions within a DNA-binding zinc finger. See, for example, Isalan, M., Klug, A. & Choo, Y. (1998) Biochemistry 37, 12026-12033; Choo, Y. & Klug, A. (1997) Curr. Opin. Str. Biol. 7, 117-125; WO 98/53060; WO 98/53059; WO 98/53058. As an example, disclosed herein is a database of approximately 200 natural human zinc fingers which have been selected (on the basis of coded contacts) as having potentially useful DNA-binding activity (see Example 1). Also disclosed in Example 1 are the predicted DNA target sequences of these zinc fingers, assigned according to the rules set out above.

As the human genome contains almost 700 zinc finger-containing proteins, there are many other candidates that can be included in a more inclusive library of natural zinc fingers. A selection of these are disclosed in Example 2.

Similar work can be carried out in other organisms, such as farm (cows, pigs, sheep, chickens, etc.), laboratory (monkeys, rats, mice, etc.) and domestic (dogs, cats, etc.) animals. In this case, it is necessary to select natural zinc finger modules from the respective genomes of such organisms. Examples of zinc finger modules which have been selected from mouse,_chicken and certain plant genomes, are disclosed in Example 3.

d. Zinc Finger Chimeric Peptides

In a preferred embodiment, the composite binding polypeptides described herein comprise chimeric nucleic acid binding polypeptides.

A chimeric nucleic acid binding polypeptide, also referred to as a fusion polypeptide, comprises a binding domain (comprising a number of nucleic acid binding polypeptide modules or fingers) designed to bind specifically to a target nucleotide sequence, together with one or more further biological effector domains or functional domains. The terms “biological effector domain” and “functional domain” refer to any polypeptide (of functional fragment thereof) that has a biological function. Included are enzymes, receptors, regulatory domains, transcriptional activation or repression domains, binding sequences, dimerisation, trimerisation or multimerisation sequences, sequences involved in protein transport, localisation sequences such as subcellular localisation sequences, nuclear localisation, protein targeting or signal sequences. Furthermore, biological effector domains may comprise polypeptides involved in chromatin remodelling, chromatin condensation or decondensation, DNA replication, transcription, translation, protein synthesis, etc. Fragments of such polypeptides comprising the relevant activity (i.e., functional fragments) are also included in this definition. Preferred biological effector domains include transcriptional modulation domains such as transcriptional activators and transcriptional repressors, as well as their functional fragments.

The effector domain(s) can be covalently or non-covalently attached to the binding domain.

Chimeric nucleic acid binding polypeptides preferably comprise transcription factor activity, for example, a transcriptional modulation activity such as transcriptional activation or transcriptional repression activity. For example, a zinc finger chimeric polypeptide may comprise a binding domain designed to bind specifically to a particular nucleotide sequence, and one or more further biological effector domains, preferably a transcriptional activation or repression domain, as described in further detail below. The zinc finger chimeric polypeptide may comprise one or more zinc fingers or zinc finger binding modules.

Preferably, in the case of a chimeric polypeptide comprising transcriptional modulation activity, a nuclear localisation domain is attached to the DNA binding domain to direct the chimeric polypeptide to the nucleus.

Generally, a chimeric nucleic acid binding polypeptide, such as a chimeric zinc finger polypeptide, can also include an effector domain to regulate gene expression. The effector domain can be directly derived from a basal or regulated transcription factor such as, for example, transactivators, repressors, and proteins that bind to insulator or silencer sequences. See, for example, Choo & Klug (1995) Curr. Opin. Biotech. 6: 431-436; Choo, Y. & Klug, A. (1997) Curr. Opin. Str. Biol. 7, 117-125; Rebar & Pabo (1994) Science 263: 671-673; Jamieson et al. (1994) Biochem. 33: 5689-5695; Goodrich et al (1996) Cell 84: 825-830; Vostrov, A. A. & Quitschke, W. W. (1997) J. Biol. Chem. 272: 33353-33359 and WO 00/41566 and references disclosed therein. Other useful domains are derived from receptors such as, for example, nuclear hormone receptors (Kumar, R & Thompson, E. B. (1999) Steroids 64: 310-319 ), and their co-activators and co-repressors (Ugai, H. et al. (1999) J. Mol. Med. 77: 481-494).

A chimeric nucleic acid binding polypeptide can also include other domains that may be advantageous within the context of the control of gene expression. Such domains include, but are not limited to, protein-modifying domains such as histone acetyltransferases, kinases, methylases and phosphatases, which can silence or activate genes by modifying DNA structure or the proteins that associate with nucleic acids. See, for example, Wolffe, Science 272: 371-372 (1996); Taunton et al., Science 272: 408-411 (1996); Hassig et al., Proc. Natl. Acad. Sci. USA. 95: 3519-3524 (1998); Wang, Trends Biochem. Sci. 19: 373-376 (1994); and Schonthal & Semin, Cancer Biol. 6: 239-248 (1995). Additional useful effector domains include those that modify or rearrange nucleic acid molecules such as methyltransferases, endonucleases, ligases, recombinases etc. See, for example, Wood, Ann. Rev. Biochem. 65: 135-167 (1996); Sadowski, FASEB J. 7: 760-767 (1993); Cheng, Curr. Opin. Struct. Biol. 5: 4-10 (1995); Wu et al. (1995) Proc. Natl. Acad. Sci. USA 92:344-348; Nahon & Raveh, Nucleic Acids Res 1998 Mar 1;26(5):1233-9; Smith et al. Nucleic Acids Res. 1999 Jan 15;27(2):674-81; and Smith et al. (2000) Nucleic Acids Res. September 1; 28(17):3361-9. It will be appreciated that the biological effector domain portion of the chimeric polypeptide may itself also comprise such activities, without the need for further additional domains.

For the purpose of gene activation, zinc finger domains may be fused to the VP64 domain. See, for example, Seipel et al., EMBO J. 11: 4961-4968 (1996). Other preferred transactivator domains include the herpes simplex virus (HSV) VP16 domain (Hagmann et al. (1997) J. Virol. 71: 5952-5962; Sadowski et al. (1988) Nature 335:563-564), transactivation domain 1 and/or domain 2 of the p65 subunit of nuclear factor-κB (NF-κB (Schmitz, M. L. et al. (1995) J. Biol. Chem. 270: 15576-15584 ). Other transcription factors are reviewed in, for example, Lekstrom-Himes J. & Xanthopoulos K. G. (C/EBP family) J. Biol. Chem. 273: 28545-28548 (1998); Bieker, J. J. et al., (globin gene transcription factors) Ann. N.Y. Acad. Sci. 850: 64-69 (1998), and Parker, M. G. (estrogen receptors) Biochem. Soc. Symp. 63: 45-50 (1998).

Use of a transactivation domain from the estrogen receptor is disclosed in Metivier, R., Petit, F G., Valotaire, Y. & Pakdel, F. (2000) Mol. Endocrinol. 14: 1849-1871. Furthermore, activation domains from the globin transcription factors EKLF (Pandya, K. Donze, D. & Townes T. (2001) J. Biol. Chem. 276: 8239-8243) may also be used, as well as a transactivation domain from FKLF (Asano, H. Li, XS. & Stamatoyannopoulos, G. (1999) Mol. Cell. Biol. 19: 3571-3579). C/EPB transactivation domains may also be employed in the methods described herein. The C/EBP epsilon activation domain is disclosed in Verbeek, W., Gombart, A F, Chumakov, A M, Muller, C, Friedman, A D, & Koeffler, H P (1999) Blood 15: 3327-3337. Kowenz-Leutz, E. & Leutz, A. (1999) Mol. Cell. 4: 735-743 disclose the use of the C/EBP tau activation domain, while the C/FBP alpha transactivation domain is disclosed in Tao, H., & Umek, R M. (1999) DNA Cell Biol. 18: 75-84.

It is known that zinc finger proteins may be fused to transcriptional repression domains such as the Kruppel-associated box (KRAB) domain to form powerful repressors. These domains are 1([own to repress expression of a reporter gene even when bound to sites a few kilobase pairs upstream from the promoter of the gene (Margolin et al., 1994, Proc. Natl. Acad. Sci. USA 91: 4509-4513). Hence, in certain embodiments, the KRAB repressor domain from the human KOX-1 protein is used to repress gene activity (Moosmann et al., Biol. Chem. 378: 669-677 (1997); Thiesen et al., New Biologist 2: 363-374 (1990)). In additional embodiments, larger fragments of the KOX-1 protein comprising the KRAB domain, up to and including full-length KOX protein, are used as transcriptional repression domains. See, for example, Abrink et al. (2001) Proc. Natl. Acad. Sci. USA 98:1422-1426. Other preferred transcriptional repressor domains are known in the art and include, for example, the engrailed domain (Han et al., EMBO J. 12: 2723-2733 (1993)), the snag domain (Grimes et al., Mol Cell. Biol. 16: 6263-6272 (1996)) and the transcriptional repression domain of v-erbA (e.g. Urnov et al. (2000) EMBO J. 19:4074-4090; Sap et al. (1989) Nature 340:242-244 and Ciana et al. (1999) EMBO J. 17:7382-7394).

Biological effector domains can be covalently or non-covalently linked to a binding domain. In one embodiment, a covalent linker comprises a flexible amino acid sequence; fusion polypeptides according to this embodiment comprise a nucleic acid binding domain fused, by an amino acid linker, to a biological effector domain. Alternatively, a covalent linker may comprise a synthetic, non-amino acid based, chemical linker, for example, polyethylene glycol. Synthetic linkers are commercially available, and methods of chemical conjugation are known in the art. Covalent linkers may comprise flexible or structured linkers, as described above.

Non-covalent linkages between a nucleic acid binding domain and an effector domain can be formed using, for example, leucine zipper/coiled coil domains, or other naturally occurring or synthetic dimerisation domains. See e.g., Luscher, B. & Larsson, L. G. Oncogene 18:2955-2966 (1999) and Gouldson, P. R. et al., Neuropsychopharmacology 23: S60-S77 (2000).

The expression of composite binding polypeptides (for example, zinc finger polypeptides) can be controlled by tissue specific promoter sequences such as, for example, the lck promoter (thymocytes, Gu, H. et al., Science 265: 103-106 (1994)); the human CD2 promoter (T-cells and thymocytes, Zhumabekov, T. et al., J. Immunological Methods 185: 133-140 (1995)); the alpha A-crystallin promoter (eye lens, Lakso, M. et al., Proc. Natl. Acad. Sci. 89: 6232-6236 (1992)); the alpha-calcium-calmodulin-dependent kinase II promoter (hippocampus and neocortex, Tsien, J. et al., Cell 87: 1327-1338 (1996)); the whey acidic protein promoter (mammary gland, Wagner, K.-U. et al., Nucleic Acids Res. 25: 4323-4330 (1997)); the aP2 enhancer/promoter (adipose tissue, Barlow C. et al., Nucleic Acids Res. 25: 2543-2545 (1997)); the aquaporin-2 promoter (renal collecting duct, Nelson R. et al., Am. J. Physiol. 275: C216-C226 (1998)); and the mouse myogenin promoter (skeletal muscle, Grieshammer, U. et al., Dev. Biol. 197: 234-247 (1998)). The expression of such polypeptides can also be controlled by inducible systems, in particular, controlled by small molecule induction such as the tetracycline-controlled systems (tet-on and tet-off), the RU-486 or tamoxifen hormone analogue systems, or the radiation-inducible early growth response gene-1 (EGR1) promoter. These promoter constructs and inducible systems have the benefit of being able to provide organ-specific and/or inducible expression of target genes for use in applications such as gene therapy and transgenic animals.

e. Vectors

The nucleic acid encoding the nucleic acid binding polypeptide such as a zinc finger polypeptide can be incorporated into intermediate vectors and transformed into prokaryotic or eukaryotic cells for expression or DNA amplification.

As used herein, vector (or plasmid) preferably refers to discrete elements that are used to introduce heterologous nucleic acid into cells for either expression or replication thereof. The term “heterologous to the cell” means that the sequence does not naturally exist in the genome of the host cell but has been introduced into the cell. The term “introduced into” means that a procedure is performed on a cell, tissue, organ or organism such that the gene encoding the nucleic acid binding polypeptide (for example, a zinc finger polypeptide) previously absent from the cell or cells is then present in the cell or cells. Alternatively, or in addition, the gene may be initially present in the cell or cells and subsequently altered by introduction of heterologous DNA. A heterologous sequence may include a modified sequence introduced at any chromosomal site, or which is not integrated into a chromosome, or which is introduced by homologous recombination such that it is present in the genome in the same position as the native allele. Selection and use of such vectors are well within the skill of the person of ordinary skill in the art. Many vectors are available, and selection of an appropriate vector will depend on the intended use of the vector, i.e. whether it is to be used for DNA amplification or for nucleic acid expression, the size of the DNA to be inserted into the vector, and the host cell to be transformed with the vector, etc. Another consideration is whether the vector is to remain episomal or integrate into the host genome. Suitable vectors may be of bacterial, viral, insect or mammalian origin. Intermediate vectors for storage or manipulation of the nucleic acid encoding the nucleic acid binding polypeptide, or for expression and purification of the polypeptide are typically of prokaryotic origin. Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. The nucleic acid binding polypeptides such as zinc finger polypeptides described here are preferably inserted into a vector suitable for expression in mammalian cells.

Prokaryote, yeast and higher eukaryote cells may be used for replicating DNA and producing the nucleic acid binding protein. Suitable prokaryotes include eubacteria, such as Gram-negative or Gram-positive organisms, such as E. coli, e.g. E. coli K-12 strains, DH5a and HB101, or Bacilli. Further hosts suitable for the vectors include eukaryotic microbes such as filamentous fungi or yeast, e.g. Saccharomyces cerevisiae. Higher eucaryotic cells include insect and vertebrate cells, particularly mammalian cells including human cells or nucleated cells from other multicellular organisms. In recent years propagation of vertebrate cells in culture (tissue culture) has become a routine procedure. Examples of useful,mammalian host cell lines are epithelial or fibroblastic cell lines such as Chinese hamster ovary (CHO) cells, NIH 3T3 cells, HeLa cells or 293T cells. The host cells referred to in this disclosure comprise cells in in vitro culture as well as cells that are within a host animal.

Each vector contains various components depending on its function (amplification of DNA or expression of DNA) and the host cell for which it is compatible. The vector components generally include, but are not limited to, one or more of the following: an origin of replication, one or more selectable marker genes, a promoter, an enhancer element, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2μ plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Advantageously, an expression and cloning vector contains a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC1 8 or pUC19, which contain both E. coli replication origin and E. coli genetic marker conferring resistance to antibiotics, such as ampicillin and tetracycline. Vectors such as these are commercially available.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Suitable selectable markers for mammalian cells are those that enable the identification of cells competent to take up nucleic acid, such as dihydrofolate reductase (DHFR, methotrexate resistance), thymidine kinase, or genes conferring resistance to neomycin, G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes the nucleic acid binding protein. Amplification is the process by which genes in greater demand (such as one encoding a protein that is critical for growth), together with closely associated genes (such as one encoding a composite binding polypeptide), are reiterated in tandem within the chromosomes of recombinant cells. Increased quantities of desired protein are usually synthesised from this amplified DNA.

Expression and cloning vectors usually contain control sequences that are recognised by the host organism and are operably linked to the nucleic acid encoding a nucleic acid binding polypeptide. The term “control sequences” is intended to include, at a minimum, components whose presence can influence expression, and can also include additional components whose presence is advantageous, for example, leader sequences and fusion partner sequences. The term “operably linked” means that the components described are in a relationship permitting them to function in their intended manner. Typical control sequences include promoters, enhancers and other expression regulation signals such as terminators. Such a promoter may be inducible or constitutive. A regulatory sequence operably linked to a coding sequence is ligated in such a way that expression of the coding sequence is achieved under conditions compatible with the control sequences.

The term promoter is well known in the art and encompasses nucleic acid regions ranging in size and complexity from minimal promoters to promoters including upstream elements and enhancers. Suitable promoters for use in prokaryotic and eucaryotic cells are well known in the art, and described in for example, Current Protocols in Molecular Biology (Ausubel et al., eds., 1994) and Molecular Cloning. A Laboratory Manual (Sambrook et al., 2^{nd} ed. 1989).

Promoters suitable for use with prokaryotic hosts include, for example, the β-lactamase and lactose promoter systems, alkaline phosphatase, the tryptophan (Trp) promoter system and hybrid promoters such as the tac promoter. Their nucleotide sequences have been published, thereby enabling the skilled worker to ligate them to DNA encoding a composite binding protein, using linkers or adapters to supply any required restriction sites. Promoters for use in bacterial systems will also generally contain an adjacent ribosome binding site (e.g., a Shine-Dalgarno sequence) operably linked to the DNA encoding the composite binding polypeptide.

Preferred expression vectors are bacterial expression vectors, which comprise a promoter of a bacteriophage such as phage lambda, SP6, T3 or T7, for example, which is capable of functioning in bacteria. In one of the most widely used expression systems, the nucleic acid encoding the fusion protein can be transcribed from a vector by T7 RNA polymerase (Studier et al, Methods in. Enzymol. 185: 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively, the polymerase gene may be introduced on a lambda phage by infection with an int⁻ phage such as the CE6 phage, which is commercially available (Novagen, Madison, Wis., USA). Other vectors include vectors containing the lambda PL promoter such as PLEX (Invitrogen, NL), vectors containing the trc promoters such as pTrcHisXpressTm (Invitrogen), or pTrc99 (Pharmacia Biotech, SE), or vectors containing the tac promoter such as pKK223-3 (Pharmacia Biotech), or PMAL (New England Biolabs, Beverly, Mass., USA). A suitable vector for expression of proteins in mammalian cells is the CMV enhancer-based vector such as pEVRF (Matthias, et al., (1989) Nucleic Acids Res. 17, 6418).

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phosphoglycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PHO5 promoter is, for example, a shortened acid phosphatase PH05 promoter devoid of the upstream regulatory elements (UAS) such as the PH05 (−173) promoter element starting at nucleotide −173 and ending at nucleotide −9 of the PH05 gene.

The promoter is typically selected from promoters which are found in animal cells, although prokaryotic promoters and promoters functional in other eukaryotic cells can be used. Typically, the promoter is derived from viral or animal gene sequences, may be constitutive or inducible, and may be strong or weak.

Viral promoters can be derived from viruses such as polyoma virus, adenoviruses, adeno-associated viruses, poxviruses (e.g., fowlpox virus), papilloma viruses (e.g., BPV), avian sarcoma virus, cytomegalovirus (CMV), herpesviruses, retroviruses, lentiviruses and simian virus 40 (SV40). An example of a relatively weak viral promoter is thymidine kinase promoter from herpes simplex virus (HSV-TK).

Mammalian derived promoters can be heterologous to the animal in which composite binding polypeptide (such as zinc finger polypeptide) expression is to occur, or they can be host sequences. In some applications it is preferable to use a promoter that is active in all cell types, however it is often preferable to use promoter sequences that are active in specific cell types only.

The actin promoter and the strong ribosomal protein promoter are examples of promoter sequences that are active in all cell types. In contrast, by using promoters that are specific for certain cell or tissue types, the gene encoding the nucleic acid binding polypeptide can be expressed only in the required cell or tissue types. This may be of extreme importance for applications such as gene therapy, and for the production of viable transgenic animals. Such promoters are known in the art and include the lck promoter (thymocytes, Gu, H. et al., Science 265: 103-106 (1994)), the human CD2 promoter (T-cells and thymocytes, Zhumabekov, T. et al., J. Immunological Methods 185: 133-140 (1995)); the alpha A-crystallin promoter (eye lends, Lakso, M. et al., Proc. Natl. Acad. Sci. 89: 6232-6236 (1992)), the alpha-calcium-calmodulin-dependent kinase II promoter (hippocampus and neocortex, Tsien, J. et al., Cell 87: 1327-1338 (1996)), the whey acidic protein promoter (mammary gland, Wagner, K.-U. et al., Nucleic Acids Res. 25: 4323-4330 (1997)), the aP2 enhancer/promoter (adipose tissue, Barlow C. et al., Nucleic Acids Res. 25: 2543-2545 (1997)), the aquaporin-2 promoter (renal collecting duct, Nelson R. et al., Am. J. Physiol. 275: C216-C226 (1998)), the mouse myogenin promoter (skeletal muscle, Grieshammer, U. et al., Dev. Biol. 197: 234-247 (1998)), retinoblastoma gene promoter (nervous system, Jiang, Z. et al., J. Biol. Chem. 276: 593-600 (2001)).

The expression of nucleic acid binding polypeptides such as zinc finger polypeptides can also be controlled by small molecule induction or other inducible systems such as the tetracycline inducible systems (tet-on and tet-off), the RU-486 or tamoxifen hormone analogue systems, or the radiation-inducible early growth response gene-1 (EGR1) promoter, all of which are commercially available. By using such inducible promoter systems, transgenic lines can be established which carry a zinc finger chimeric polypeptide but express it only after addition of an inducer molecule. Thus the genes encoding the zinc finger polypeptides or other nucleic acid binding polypeptides can be expressed (or not expressed) in response to the small molecule, which can be easily administered. These systems may also allow the time and amount of polypeptide expression to be regulated.

Expression vectors typically contain expression cassettes that carry all the additional elements required for efficient expression of the nucleic acid in the host cell. Additional elements are enhancer sequences, polyadenylation and transcriptional termination signals, ribosome binding sites, and translational termination sequences.

Transcription of DNA by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (approx. bp 100-270) and the CMV early promoter enhancer. The enhancer may be spliced into the vector at a position 5′ or 3′ to the gene encoding the zinc finger polypeptide or nucleic acid binding polypeptide, but is preferably located at a site 5′ from the promoter.

It has also been shown that the expression of a heterologous gene in an animal cell may be enhanced by retaining intron sequences (as opposed to using a cDNA clone). For example, intron 1 of the human CD2 gene has been shown to enhance the level of expression of CD2 in human cells (Festenstein, R. et al. 1996 Science 271: 1123).

Advantageously, a eukaryotic expression vector encoding a nucleic acid binding protein may comprise a locus control region (LCR). LCRs are capable of directing high-level integration site-independent expression of transgenes integrated into host cell chromatin. This is particularly important where the gene encoding the zinc finger polypeptide or the nucleic acid binding polypeptide is to be expressed over extended periods of time, for applications such as transgenic animals and gene therapy, as gene silencing of integrated heterologous DNA—especially of viral origin—is known to occur (Palmer, T. D. et al., Proc. Natl. Acad. Sci. USA 88: 1330-1334 (1991); Harpers, K. et al., Nature 293: 540-542 (1981); Jahner, D. et al., Nature 298: 623-628 (1992); and Chen, W. Y. et al., Proc. Natl. Acad. Sci. USA 94: 5798-5803 (1997)). Typical LCRs are exemplified by the human β-globin cluster, and the HS-40 regulatory region from the α-globin locus.

Eukaryotic vectors may also contain sequences necessary for the termination of transcription and for stabilising the mRNA transcript. Such sequences are commonly available from the 5′ and 3′ untranslated regions of eukaryotic or viral DNAs, and are known in the art. These regions contain nucleotide segments transcribed as polyadenylated fragments in the untranslated portion of the mRNA encoding the relevant polypeptide. An appropriate terminator of transcription is fused downstream of the gene encoding the selected nucleic acid binding polypeptide such as a zinc finger protein. Any of a number of known transcriptional terminator, RNA polymerase pause sites and polyadenylation enhancing sequences can be used at the 3′ end of the nucleic acid encoding for example a zinc finger polypeptide (see, for example, Richardson, J. P. Crit. Rev. Biochem. Mol. Biol. 28:1-30 (1993); Yonaha M. & Proudfoot, N. J. EMBO J. 19: 3770-3777 (2000); Ashfield, R. et al., EMBO J. 10: 4197-4207 (1991); Hirose, Y. & Manley, J. L. Nature 395: 93-96 (1998)).

The nucleic acid binding polypeptides are generally targeted to the cell nucleus so that they are able to interact with host cell DNA and bind to the appropriate DNA target in the nucleus and regulate transcription. To effect this, a nuclear localisation sequence (NLS) is incorporated in frame with the expressible nucleic acid binding polypeptide (e.g., zinc finger polypeptide) gene construct. The NLS can be fused either 5′ or 3′ to the sequence encoding the binding protein, but preferably it is fused to the C-terminus of the chimeric polypeptide.

The NLS of the wild-type Simian Virus 40 Large T-Antigen (Kalderon et al. (1984) Cell 37: 801-813; and Markland et al. (1987) Mol. Cell. Biol. 7: 4255-4265) is an appropriate NLS and provides an effective nuclear localisation mechanism in animals. However, several alternative NLSs are known in the art and can be used instead of the SV40 NLS sequence. These include the NLSs of TGA-1A and TGA-1B.

Composite binding polypeptides can comprise tag sequences to facilitate studies and/or preparation of such molecules. Tag sequences may include FLAG-tags, myc-tags, 6his-tags, hemagglutinin tags or any other suitable tag known in the art.

Moreover, the nucleic acid binding protein gene according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate.

Construction of vectors employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing nucleic acid binding protein expression and function are known to those skilled in the art. Gene presence, amplification and/or expression may be measured in a sample directly, for example, by conventional Southern blotting, Northern blotting to quantify the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

f. Applications of Composite Binding Polypeptides

Nucleic acid binding proteins according to the invention can be employed in a wide variety of applications, including diagnostics and as research tools, and also in therapeutic applications and in transgenic organisms.

In Vitro Applications

Poly-zinc finger peptides of this invention may be employed as diagnostic tools for identifying the presence of nucleic acid molecules in a complex mixture. Nucleic acid binding molecules according to the invention can differentiate single base pair changes in target nucleic acid molecules.

Accordingly, the invention provides methods for determining the presence of a target nucleic acid molecule, wherein the target nucleic acid molecule comprises a target sequence, comprising the steps of:

a) preparing a nucleic acid binding protein, by a method set forth above, which is specific for the target nucleic acid sequence;

b) exposing a test system to the nucleic acid binding protein under conditions which promote binding of the protein to the target sequence, and removing any nucleic acid binding protein which remains unbound;

c) testing for the presence of the nucleic acid binding protein in the test system; wherein, if the nucleic acid binding protein is detected, the target nucleic acid molecule is present and, if the nucleic acid binding protein is not detected, the target nucleic acid molecule is not present. In additional embodiments, quantitation of the amount of nucleic acid binding protein allows quantitation of the amount of the target nucleic acid molecule present in the test system.

In a preferred embodiment, the nucleic acid binding molecules of the invention can be incorporated into an ELISA assay. For example, phage displaying composite binding polypeptides can be used to detect the presence of the target nucleic acid, and visualised using enzyme-linked anti-phage antibodies.

Further improvements to the use of phage expressing a composite binding polypeptide for diagnosis can be made, for example, by co-expressing a marker protein fused to the minor coat protein (gVIII) of a filamentous bacteriophage. Since detection with an anti-phage antibody would then be unnecessary, the time and cost of each diagnosis would be further reduced. Depending on the requirements, suitable markers for display might include fluorescent proteins (A. B. Cubitt, et al., (1995) Trends Biochem. Sci. 20, 448-455; T. T. Yang, et al., (1996) Gene 173, 19-23), or an enzyme such as alkaline phosphatase (J. McCafferty, R. H. Jackson, D. J. Chiswell, (1991) Protein Engineering 4, 955-961). Labelling different types of diagnostic phage with distinct markers would allow multiplex screening of a single nucleic acid sample. Nevertheless, even in the absence of such refinements, the basic ELISA technique is reliable, fast, simple and particularly inexpensive. Moreover it requires no specialised apparatus, nor does it employ hazardous reagents such as radioactive isotopes, making it amenable to routine use in the clinic. The major advantage of the protocol is that it obviates the requirement for gel electrophoresis, and so opens the way to automated nucleic acid diagnosis.

The invention provides nucleic acid binding proteins that have exquisite specificity. The invention lends itself, therefore, to the design of any molecule of which specific nucleic acid binding is required. For example, the proteins according to the invention may be employed in the manufacture of chimeric restriction enzymes, in which a nucleic acid cleaving domain is fused to a nucleic acid binding domain comprising a zinc finger as described herein.

In Vivo Applications

The invention further provides composite binding polypeptides (and nucleic acids encoding them) that may be used in transgenic organisms (such as non-human animals), as therapeutic agents, and in gene therapy applications.

A transgenic animal is an animal, preferably a non-human animal, containing at least one foreign gene, called a transgene, in its genetic material. Preferably, the transgene is contained in the animal's germ line such that it can be transmitted to the animal's offspring. Transgenic animals may carry the transgene in all their cells or may be genetically mosaic.

Constructs useful for creating transgenic animals according to the invention comprise genes encoding nucleic acid binding polypeptides, optionally under the control of nucleic acid sequences directing their expression in cells of a particular lineage. Alternatively, nucleic acid binding polypeptide encoding constructs may be under the control of non-lineage-specific promoters, and/or inducibly regulated. Typically, DNA fragments on the order of 10 kilobases or less are used to construct a transgenic animal (Reeves, 1998, New. Anat., 253:19). A transgenic animal expressing one transgene can be crossed to a second transgenic animal expressing second transgene such that their offspring will carry both transgenes.

Although the majority of previous studies have involved transgenic mice, other species of transgenic animal have also been produced, such as rabbits, sheep, pigs (Hammer et al., 1985, Nature 315:680-683; Kumar, et al., U.S. 05922854; Seebach, et al., U.S. Pat. No. 6,030,833) and chickens (Salter et al., 1987, Virology 157:236-240). Transgenic animals are currently being developed to serve as bioreactors for the production of useful pharmaceutical compounds (Van Brunt, 1988, Bio/Technology 6:1149-1154; Wilmut, et al., 1988, New Scientist (July 7 issue) pp. 56-59). Up-regulation of endogenous or exogenous genes expressing useful polypeptides, such as therapeutic polypeptides, by means of a heterologous nucleic acid binding polypeptide, may be used to produce such polypeptides in transgenic animals. Preferably, the polypeptides are secreted into an extractable fluid, such as blood or mammary fluid (milk), to enable easy isolation of the polypeptide.

Furthermore, the invention provides the use of polypeptide fusions comprising an integrase, such as a viral integrase, and a nucleic acid binding protein according to the invention to target nucleic acid sequences in vivo (Bushman, (1994) PNAS (USA) 91:9233-9237). In gene therapy applications, the method may be applied to the delivery of functional genes into defective genes, or the delivery of a heterologous nucleic acid in order to disrupt an endogenous gene. Alternatively, genes may be delivered to known, repetitive stretches of nucleic acid, such as centromeres, together with an activating sequence such as an LCR. This would represent a route to the safe and predictable incorporation of nucleic acid into the genome.

In conventional therapeutic applications, nucleic acid binding proteins according to this embodiment may be used to specifically eliminate cells having mutant vital proteins. For example, if a mutant ras gene is targeted, cells comprising this mutant gene will be destroyed because ras is essential to cellular survival. Alternatively, the action of transcription factors can be modulated, preferably reduced, by administering to the cell agents which bind to the binding site specific for the transcription factor. For example, the activity of HIV tat may be reduced by binding proteins specific for HIV TAR.

Moreover, binding proteins according to the invention can be coupled to toxic molecules, such as nucleases, which are capable of causing irreversible nucleic acid damage and cell death. Such agents are capable of selectively destroying cells that comprise a mutation in their endogenous nucleic acid.

Nucleic acid binding proteins and derivatives thereof as set forth above may also be applied to the treatment of infections and the like in the form of organism-specific antibiotic or antiviral drugs. In such applications, the binding proteins can be coupled to a nuclease or other nuclear toxin and targeted specifically to the nucleic acids of microorganisms.

Transgenic animals comprising transgenes, optionally integrated within the genome, and expressing heterologous zinc finger and other nucleic acid binding polypeptides from transgenes, may be created by a variety of methods. Methods for producing transgenic animals are known in the art, and are described by Gordon, J. & Ruddle, F. H. Science 214: 1244-1246 (1981); Jaenisch, R. Proc. Natl. Acad. Sci. USA 73: 1260-1264 (1976); Gossler et al., (1986) Proc. Natl. Acad. Sci. USA 83:9065-9069; Hogan et al., Manipulating the Mouse Embryo: A Laboratory Manual, (1988); and U.S. Pat. Nos. 5,175,384; 5,434,340 and 5,591,669.

Pharmaceutical Preparations

The invention likewise relates to pharmaceutical preparations which contain the compounds according to the invention or pharmaceutically acceptable salts thereof as active ingredients, and to processes for their preparation.

The pharmaceutical preparations according to the invention which contain the compound according to the invention or pharmaceutically acceptable salts thereof are those for enteral, such as oral, furthermore rectal, and parenteral administration to (a) warm-blooded animal(s), the pharmacological active ingredient being present on its own or together with a pharmaceutically acceptable carrier. The daily dose of the active ingredient depends on the age and the individual condition and also on the manner of administration.

The novel pharmaceutical preparations contain, for example, from about 10% to about 80% (or any integral percentage therebetween), preferably from about 20% to about 60%, of the active ingredient. Pharmaceutical preparations according to the invention for enteral or parenteral administration are, for example, those in unit dose forms, such as sugar-coated tablets, tablets, capsules or suppositories, and furthermore ampoules. These are prepared in a manner known per se, for example by means of conventional mixing, granulating, sugar-coating, dissolving or lyophilising processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

Suitable carriers are, in particular, fillers, such as sugars, for example lactose, sucrose, mannitol or sorbitol, cellulose preparations and/or calcium phosphates, for example tricalcium phosphate or calcium hydrogen phosphate, furthermore binders, such as starch paste, using, for example, corn, wheat, rice or potato starch, gelatin, tragacanth, methylcellulose and/or polyvinylpyrrolidone, if desired, disintegrants, such as the abovementioned starches, furthermore carboxymethyl starch, crosslinked polyvinylpyrrolidone, agar, alginic acid or a salt thereof, such as sodium alginate; auxiliaries are primarily glidants, flow-regulators and lubricants, for example silicic acid, talc, stearic acid or salts thereof, such as magnesium or calcium stearate, and/or polyethylene glycol. Sugar-coated tablet cores are provided with suitable coatings which, if desired, are resistant to gastric juice, using, inter alia, concentrated sugar solutions which, if desired, contain gum arabic, talc, polyvinylpyrrolidone, polyethylene glycol and/or titanium dioxide, coating solutions in suitable organic solvents or solvent mixtures or, for the preparation of gastric juice-resistant coatings, solutions of suitable cellulose preparations, such as acetylcellulose phthalate or hydroxypropylmethylcellulose phthalate. Colorants or pigments, for example to identify or to indicate different doses of active ingredient, may be added to the tablets or sugar-coated tablet coatings.

Other orally utilisable pharmaceutical preparations are hard gelatin capsules, and also soft closed capsules made of gelatin and a plasticiser, such as glycerol or sorbitol. The hard gelatin capsules may contain the active ingredient in the form of granules, for example in a mixture with fillers, such as lactose, binders, such as starches, and/or lubricants, such as talc or magnesium stearate, and, if desired, stabilisers. In soft capsules, the active ingredient is preferably dissolved or suspended in suitable liquids, such as fatty oils, paraffin oil or liquid polyethylene glycols, it also being possible to add stabilisers.

Suitable rectally utilisable pharmaceutical preparations are, for example, suppositories, which consist of a combination of the active ingredient with a suppository base. Suitable suppository bases are, for example, natural or synthetic triglycerides, paraffin hydrocarbons, polyethylene glycols or higher alkanols. Furthermore, gelatin rectal capsules which contain a combination of the active ingredient with a base substance may also be used. Suitable base substances are, for example, liquid triglycerides, polyethylene glycols or paraffin hydrocarbons.

Suitable preparations for parenteral administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilisers.

The dose of the active ingredient depends on the warm-blooded animal species, the age and the individual condition and on the manner of administration. For example, an approximate daily dose of about 10 mg to about 250 mg is to be estimated in the case of oral administration for a patient weighing approximately 75 kg.

g. Transformation and Transfection

DNA can be stably incorporated into cells or can be transiently expressed using methods known in the art and described below. Stably transfected cells can be prepared by transfecting cells with an expression vector containing a selectable marker gene, and growing the transfected cells under conditions selective for cells expressing the marker gene. To prepare transient transfectants, cells are transfected with a reporter gene to monitor transfection efficiency.

There are many well-known methods of introducing foreign nucleic acids into host cells, which include electroporation, calcium phosphate co-precipitation, particle bombardment, microinjection, naked DNA, liposomes, lipofection, and viral infection etc (see, e.g. Sambrook et al. (1989) Molecular Cloning: A Laboratory Manual, Second Edition, Cold Spring Harbor Laboratory Press, and Mountain, A. Trends Biotechnol. 18: 119-128 (2000) for a review). Any of the above methods can be used, as long as it is compatible with the host cell. Linear nucleic acid molecules have been found to be more efficiently incorporated into mammalian genomes than circular plasmids. Additionally, nucleic acid molecules may be delivered to specific target tissues or to individual cells. Viral based gene transfer is often favoured for introducing nucleic acids into mammalian cells and specific target tissues, and several viral delivery approaches are in clinical trials for gene therapy applications. However, non-viral methods are attractive due to their greater safety for the purpose of gene transfer to humans.

The preferred methods of particle bombardment use biolistics made from gold (or tungsten). Compared with other transfection procedures, particle bombardment requires a low amount of nucleic acid and a smaller number of cells, making the procedure generally more efficient (Heiser, W. C. Anal. Biochem. 217: 185-196 (1994); Klein, T. M. & Fitzpatrick-McElligott, S. Curr. Opin. Biotechnol. 4: 583-590 (1993)). The procedure is particularly suited for organisms that are difficult to transfect, and for introducing DNA into organelles, such as mitochondria and chloroplasts. Although generally used for ex vivo applications, the procedure is also suitable for in vivo transfection of skin tissue. Suitable methods are known in the art and described, for instance, in U.S. Pat. Nos. 5,489,520 and 5,550,318. See also, Potrykus (1990) Bio/Technol. 8: 535-542; and Finnegan et al. (1994) Bio/Technol. 12: 883-888.

Microinjection is a common method of nucleic acid delivery to isolated cells (Palmiter, R. D. & Brinster, R. L. Annu. Rev. Genet. 20: 465-499 (1986); Wall, R. J. et al., J. Cell Biochem. 49: 113-120 (1992); Chan, A. W. et al., Proc. Natl. Acad. Sci. USA 95: 14028-14033 (1998)). DNA is generally injected into cells and the cells may then be re-introduced into animals. Procedures for such a technique are described in U.S. Pat. Nos. 5,175,384 and 5,434,340, and improvements to the technique are described in WO 00/69257.

Efficient for gene transfer in vivo can be obtained following local injection of naked DNA. While expression of injected DNA in skin lasts for only a few days, injected DNA in mouse skeletal muscle has been shown to last for up to nine months (Wolff, J. A. et al., Hum. Mol. Genet. 1: 363-369 (1992)). Naked DNA is particularly suited to gene therapy for preventive and therapeutic vaccines.

Cationic liposomes containing cholesterol are particularly suited for delivery of nucleic acids to humans as they are biodegradable and stable in the bloodstream. Liposomes can be injected intravenously, subcutaneously or inhaled as an aerosol. Stribling et al. (1992) Proc. Natl. Acad. Sci. USA 89:11,277-11,281. Liposomes can be targeted to certain cell types by incorporating ligands, receptors or antibodies (immunolipids) into the lipid membrane (US. Pat. No. 4,957,773). On contacting target cells, entry of DNA from liposomes is via endocytosis and diffusion. Preparations of lipid formulations are commercially available and methods for their use are well documented (Bogdanenko, E. V. et al., Vopr. Med. Khim. 46: 226-245 (2000); Natsume, A. et al., Gene Ther. 6: 1626-1633 (1999)).

Uptake of DNA into animal cells can also be enhanced by using transfection agents. “Transfecting agent”, as utilised herein, means a composition of matter added to the genetic material for enhancing the uptake of exogenous DNA segment (s) into a eukaryotic cell, preferably a mammalian cell, and more preferably a mammalian germ cell. The enhancement is measured relative to the uptake in the absence of the transfecting agent. Examples of transfecting agents include adenovirus-transferring-polylysine-DNA complexes. These complexes generally augment the uptake of DNA into the cell and reduce its breakdown during its passage through the cytoplasm to the nucleus of the cell. These complexes can be targeted to the male germ cells using specific ligands which are recognised by receptors on the cell surface of the germ cell, such as the c-kit ligand or modifications thereof. Other preferred transfecting agents include lipofectin™, lipofectamine™, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3 phosphoethanolamnine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecylN, N dihydroxyethylammonium bromide), polybrene, or poly (ethylenimine) (PEI). For example, Baneree, R. et al., Novel series of non-glycerol-based cationic transfection lipids for use in liposomal gene delivery, J. Med. Chem. 42 (21): 4292-99 (1999); Godbey, W. T. et al., Improved packing of poly (ethylenimine)-DNA complexes increases transfection efficiency, Gene Ther. 6 (8): 1380-88 (1999); Kichler, A et al., Influence of the DNA complexation medium on the transfection efficiency of lipospermine/DNA particles, Gene Ther. 5 (6): 855-60 (1998); Birchaa, J. C. et al., Physico-chemical characterisation and transfection efficiency of lipid-based gene delivery complexes, Int. J Pharm. 183 (2): 195-207 (1999). These non-viral agents have the advantage that they facilitate stable integration of xenogeneic DNA sequences into the vertebrate genome, without size restrictions commonly associated with virus-derived transfecting agents.

The most critical issues for applications such as gene therapy are the efficient delivery and appropriate expression of transgenes in host cells. For this purpose, viral systems are particularly well suited as viruses have evolved to efficiently cross the plasma membrane of eukaryotic cells and express their nucleic acids in host cells. Suitability of viral vectors is assessed primarily on their ability to carry foreign nucleic acids and deliver and express transgenes with high efficiency. Current applications utilise both RNA and DNA virus based systems, and 70% of gene therapy trials use viral vectors derived from retroviruses, adenovirus, adeno-associated virus, herpesvirus and pox virus. See, for example, Flotte et al. (1995) Gene Ther. 2:357-362; Glorioso et al. (1995) Ann. Rev. Microbiol. 49:675-710; Smith (1995) Ann. Rev. Microbiol. 49:807-838; Prince (1998) Pathology 30:335-347; and Robbins et al. (1998) Pharmacol. Ther. 80:35-47. Retroviruses represent the most prominent gene delivery system as they mediate high gene transfer and expression of therapeutic genes. Members of the DNA virus family such as adenovirus, adeno-associated virus or herpesvirus are popular due to their efficiency of gene delivery. Adenoviral vectors are particularly suited when transient transfection of nucleic acid is preferred. Retroviruses express particular envelope proteins that bind to specific cell surface receptors on host cells, in order for the virus to enter the cell. Hence, the type of viral vector used should be determined by the tissue type to be targeted. See e.g., Domburg (1995) Gene Ther. 2:301-310; Gunzburg, et al. (1996) J. Mol. Med. 74:171-182; Vile et al. (1996) Mol. Biotechnol. 5:139-158; Miller (1997) “Development and Applications of Retroviral Vectors” Cold Spring Harbor Laboratory Press, Cold Spring Harbor, New York; Karavanas et al. (1998) Crit. Rev. Oncol. Hematol. 28:7-30; Hu et al. (2000) Pharmacol. Rev. 52: 493-51 1; and Walther et al. (2000) Drugs 60: 249-271 for reviews.

Safety is a critical issue for viral based gene delivery because most viruses are either pathogens or have pathogenic potential. Generally, when a replication-competent virus infects an animal cell it can express viral genes and release many new infectious viral particles in the host organism. Hence, it is very important that during transgene delivery the host animal does not receive a pathogenic virus with full replication potential. For this reason, viral-host cell systems have been developed for gene therapy treatments to prevent the creation of replication-competent viruses. In this method, viral components are divided between a vector and a helper construct to limit the ability of the virus to replicate (Miller 1997). The viral vector contains the gene(s) of interest and cis-acting elements that allow gene expression and replication, but contain deletions of some or all of the viral proteins. Helper cells (or occasionally, helper virus) are engineered to express the viral proteins needed to propagate the viral vectors. These new viral particles are able to infect target cells, reverse transcribe the vector RNA and integrate its DNA copy into the genome of the host, which can then be expressed. However, the vector can not express the viral proteins required to create new infectious particles. Helper cell lines are known in the art (see Hu, W-S & Pathak, V. K. Pharmacol. Rev. 52: 493-511 (2000), for a review).

In general, retroviral vectors are able to package reasonably long stretches of foreign DNA (up to 10 kb). Oncoviruses are a type of retrovirus, which only infect rapidly dividing cells. For this reason they are especially attractive for cancer therapy. Murine leukaemia virus (MLV)-based vectors are the most commonly used of this class. Spleen necrosis virus (SNV), Rous sarcoma virus and avian leukosis virus are other types. Lentiviral vectors are retroviral vectors that can be propagated to produce high viral titres and are able to infect non-dividing cells. They are more complex than oncoviruses and require regulation of their replication cycle. Lentiviral vectors which may be used include human immunodeficiency virus (HIV-1 and -2) and simian immunodeficiency virus (SIV) based systems. HIV infects cells of the immune system, most importantly CD4⁺ T-lymphocytes, and so may be useful for targeted gene therapy of this cell type. Another type of retrovirus is the spumavirus. Spumaviruses are attractive because of their apparent lack of toxicity. Linial (1999) J. Virol. 73:1747-1755.

Adenoviral vectors have high transduction efficiency and are able to transfect a number of different cell types, including non-dividing cells. They have a high capacity for foreign DNA and can carry up to 30 kb of non-viral DNA (for a review see, Kochanek, S. Hum. Gene Ther. 10: 2451-2459 (1999)). Recombinant adenoviral (rAd) vectors are becoming one of the most powerful gene delivery systems available and have been used to deliver DNA to post-mitotic neurons of the central nervous system (CNS) (Geddes, 13. J. et al., Front. Neuroendocrinol. 20: 296-316 (1999), and are used to treat diseases such as colon cancer (Alvarez et al., Hum. Gene Ther. 5: 597-613 (1997). Adeno-associated virus (AAV) vectors and recombinant AAV (rAAV) vectors are proving themselves to be safe and efficacious for the long-term expression of proteins to correct genetic disease. Snyder, R. O. J. (Gene. Med. 1: 166-175 (1999)) provides a review of gene delivery approaches using such vectors. Construction of such vectors is described in, for example, Samulski et al., J. Virol. 63: 3822-3828 (1989), and U.S. Pat. No. 5,173,414.

Many gene therapy trials have been conducted and are underway (over 3,500 people have been treated with gene therapy systems), and several reviews can be studied for details of the protocols and results (Hwu & Rosenberg, Ann NY Acad Sci. 1994 May 31;716:188-97; Blaese, Hosp Pract (Off Ed). 1995 Nov. 15;30(11):33-40; Blaese, Hosp Pract (Off Ed). 1995 Dec. 15;30(12):37-45; Breau & Clayman, Curr Opin Oncol. 1996 May; 8(3):227-31; Dunbar Annu Rev Med. 1996;47:11-206; Lotze Cancer J Sci Am. 1996 March;2(2):63). The first gene therapy trial was carried out by Blaese et al., (1995), to correct a genetic disorder known as adenosine deaminase (ADA) deficiency, which leads to severe immunodeficiency. Several cancer gene therapy strategies are being developed, which involve eliminating cancer cells by suicide therapy (Oldfield et al., Hum Gene Ther. 1993 February;4(1):39-69), modification of cancer cells to promote immune responses (Lotze et al., Hum Gene Ther. 1994 January;5(1):41-55), and reversion by delivery of a tumor suppressor gene (Roth et al., Hum Gene Ther. 1996 May 1 ;7(7):861-74). Another successful gene therapy trial has been conducted to combat graft-versus-host disease, which can result following transplant procedures such as bone marrow transplants (Bonini et al., Science. 1997 Jun. 13;276(5319):1719-24). This procedure was carried out using an HSV-based vector. Several gene therapy treatments are under investigation for the treatment of HIV-1 infection. Most treatments involve modification of lymphocytes, ex vivo, to suppress the expression of viral genes, by means of ribozymes, antisense RNA, mutant trans-dominant regulatory proteins and modification to elicit a host immune response (Nabel et al., Cardiovasc Res. 1994 April;28(4):445-55; Galpin et al., Hum Gene Ther. 1994 August;5(8):997-1017; Morgan R A, Walker R. Hum Gene Ther 1996 Jun. 20;7(10):1281-306 Gene therapy for AIDS using retroviral mediated gene transfer to deliver HIV-1 antisense TAR and transdominant Rev protein genes to syngeneic lymphocytes in HIV-1 infected identical twins; Wong-Staal et al., Hum Gene Ther. 1998 Nov. 1 ;9(16):2407-25). Vectors currently in use for gene therapy treatments and animal tests include those derived from Moloney murine leukemia virus, such as MFG and derivative thereof, and the MSCV retroviral expression system (Clontech, Palo Alto, Calif.). Many other vectors are also commercially available.

Viral vectors are especially important in applications when a specific tissue type is to be targeted, such as for gene therapy applications. There are two available methods for targeting genes to specific cell or tissue type. One strategy is designed to control expression of the required gene using a tissue specific promoter (discussed above), and another strategy is to control viral entry into cells. Viruses tend to enter specific cell types according to the envelope proteins that they express. However, by engineering the envelope proteins to express specific proteins as fusions, such as erythropoietin, insulin-like growth factor I and single chain variable fragment antibodies, viral vectors can be targeted to specific cell-types (Kasahara et al., Science. 1994 Nov. 25;266(5189):1373-6; Somia et al., Proc Natl Acad Sci USA. 1995 Aug. 1;92(16):7570-4; Jiang et al., J Virol. 1998 December;72(12):10148-56; Chadwick et al., J Mol Biol. 1999 Jan. 15;285(2):485-94).

In one example of tissue specific targeting in transgenic mice, a novel transgene delivery system has been developed in which the target tissue type expresses an avian viral receptor (TVA), under the control of a tissue specific promoter. Transgenic mice expressing the TVA receptor are then infected with avian leukosis virus, carrying the transgene(s) of interest (Fisher, G. H. et al., Onogene 18: 5253-5260 (1999).

h. Construction of Zinc Finger Libraries

Zinc finger libraries may be constructed from naturally-occurring human zinc finger modules. Thus, the invention provides libraries of zinc finger modules. Module libraries according to the invention may be assembled combinatorially into zinc finger polypeptides. The combinatorial assembly may be carried out biologically, using random assembly and selection technologies, or in a directed manner under computer control, assembling desired modules to produce zinc fingers having defined or random specificity. In accordance with the invention, libraries may be constructed entirely from natural zinc finger polypeptide modules from which zinc finger polypeptides having any desired specificity may be isolated. The invention, in its most preferred aspect, does not require the engineering of the specificity of any zinc finger module in order to produce a zinc finger polypeptide having specificity for any desired nucleic acid sequence.

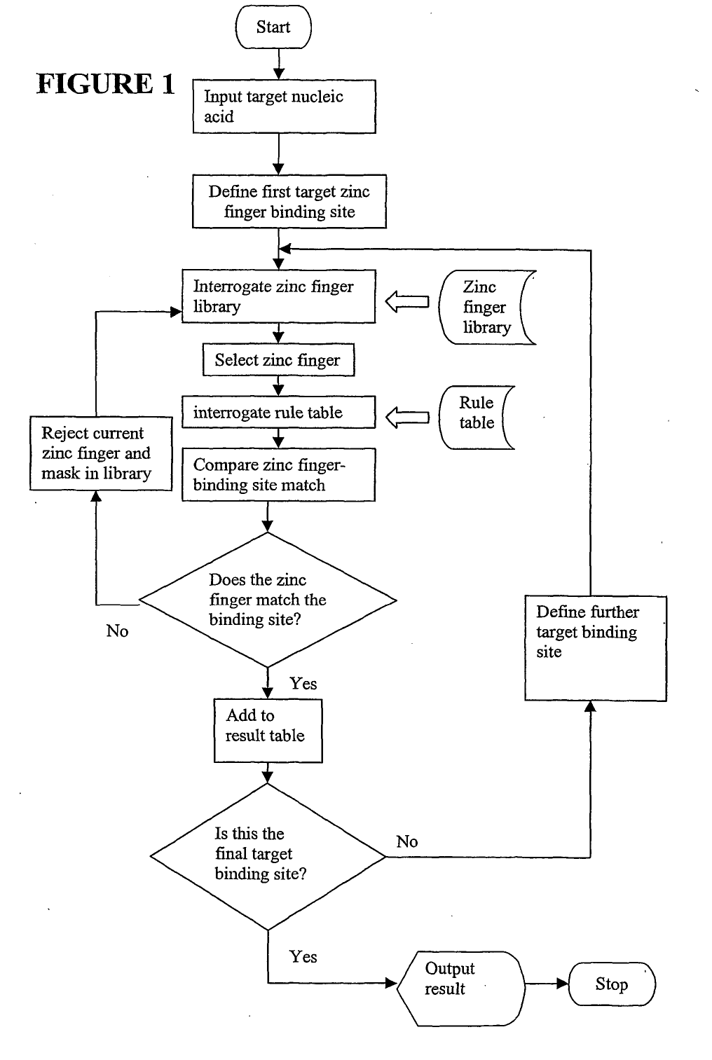

Selection of appropriate zinc finger modules for assembly into libraries of composite binding polypeptides having a predetermined binding specificity can be accomplished by applying the rules for zinc finger binding specificity set forth herein. In the case of zinc finger assembly under computer control, a rule table may be used to select zinc fingers for binding to the target site. FIG. 1 shows a flowchart depicting part of the logic used in the selection of zinc fingers from a natural library in accordance with the invention. The logic set forth in FIG. 1 may be supplemented, for example using Rules relating to zinc finger overlap. Functional testing of zinc fingers for binding to the desired binding site may be implemented in an automated fashion and integrated with the zinc finger design system.

The invention thus provides libraries of zinc finger modules. In one embodiment, the modules are human zinc finger modules. Preferably, the modules are DNA-binding zinc finger modules.

In a preferred aspect the invention provides a library of DNA-binding human zinc finger modules as set out in Example 1 below. Moreover, the invention provides a library of human zinc finger modules as set forth in Example 2 below. Sub-libraries can be prepared from either of the libraries of the invention.

The invention furthermore encompasses libraries in which zinc finger modules as set forth in Examples 1 or 2 herein are combined with other zinc finger modules to provide further libraries that may be used to generate zinc finger polypeptides.

In a still further aspect, the invention relates to libraries derived from animals other than humans, for use in said organisms in order to derive some or all of the same advantages as may be obtained with human zinc fingers for use in humans. Example 3 sets forth databases of zinc fingers from mouse, chicken and plants. Sequences of zinc fingers can be identified in other organisms by the same means, i.e. by analysis of sequence information and identification of zinc fingers in accordance with the guidance given herein.

### EXAMPLES

### Example 1

### List of Selected Human DNA-Binding Zinc Fingers

These fingers have been selected from the human genome on the basis of a prediction that they have a DNA-binding potential. This prediction is based on coded contacts (WO 96/06166, WO 98/53057, WO 98/53058; WO 98/53059 and WO 98/53060); accordingly, for each peptide unit, a 3-nucleotide DNA target subsite is shown, as the preferred sequence to which the zinc finger binds. Hence, by constructing 2- or 3-finger libraries from these 200 or so units, in the manner described in the Examples infra, there exists the potential to screen a large variety of novel DNA target sites. Note that the predicted DNA target subsites listed below are merely intended to be a guide to the DNA-binding potential. It is anticipated that, in practice, an even wider range of DNA sequences can be targeted using a library engineered from this database, through the exertion of a positive selection pressure in the library screening system.

The fingers listed below are in a format that can be linked with classical wild-type canonical “TGEKP” (SEQ ID NO:3) linkers (i.e . . . TGEKP— zinc finger peptide sequence -TGEKP— zinc finger peptide sequence -TGEKP— etc . . . ). For each peptide sequence, an oligonucleotide is designed to encode the peptide sequence; the oligonucleotide can then be linked into a library selection system, as described in the Examples infra.

Database of predicted human
DNA-binding zinc fingers
227 finger units

| Zinc | DNA | SEQ | |
|---|---|---|---|
| finger | site | ID NO | Peptide sequence |
| ZIF268 F1 | GCG | 31 | YACPVESCDRRFSRSDELTRHIRIH |
| ZIF268 F2 | TGG | 32 | FQCRICMRNFSRSDHLSTHIRTH |
| ZIF268 F3 | GCG | 33 | FACDICGRKFARSDERKRHTKIH |
| Kr-like13 | NGT | 34 | HKCHYAGCEKVYGKSSHLKAHLRTH |
| MAZ F1 | AGG | 35 | YQCPVCQQRFKRKDRMSYHVRSH |
| MAZ F2 | TGG | 36 | YNCSHCGKSFSRPDHLNSHVRQVH |
| MAZ F3 | NGT | 37 | FKCEKCEAAFATKDRLRAHTVRH |
| TIEG2 | GGG | 38 | FVCPVCDRRFMRSDHLTKHARRH |
| (SP1) F3 | | | |
| SP1 F1 | GGG | 39 | HKCHYAGCEKVYGKSSHLKAHLRTH |
| SP1 F2 | GCG | 40 | FACSWQDCNKKFARSDELARHYRTH |
| SP1 F3 | GGG | 41 | FSCPICEKRFMRSDHLTKHARRH |
| WT1 F1 | TGT | 42 | FMCAYPGCNKRYFKLSHLQMHSRKH |
| WT1 F2 | GAG | 43 | YQCDFKDCERRFSRSDQLKRHQRRH |
| WT1 F3 | TGG | 44 | FQCKTCQRKFSRSDHLKTHTRTH |
| WT1 F4 | GCG | 45 | FSCRWPSCQKKFARSDELVRHHNMH |
| TYY1 | TAT | 46 | FQCTFEGCGKRFSLDFNLRTHVRIH |
| TYY1 | NAA | 47 | YVCPFDGCNKKFAQSTNLKSHILTH |
| TF3A | GGG | 48 | FVCDYEGCGKAFIRDYHLSRHILTH |
| TF3A | GGC | 49 | FKCTQEGCGKHFASPSKLKRHAKAH |
| MAZ | GGC | 50 | HACEMCGKAFRDVYHLNRHKLSH |
| GLI1 | GCA | 51 | YMCEHEGCSKAFSNASDRAKHQNRTH |
| ZIC3 | GCA | 52 | FKCEFEGCDRRFANSSDRKKHMHVH |
| SP4 | NGG | 53 | HICHIEGCGKVYGKTSHLRAHLRWH |
| SP2 | NTG | 54 | HVCHIPDCGKTFRKTSLLRAHVRLH |
| BTE1 | NGG | 55 | HKCPYSGCGKVYGKSSHLKAHYRVH |
| GLI2 | TAG | 56 | HKCTFEGCSKAYSRLENLKTHLRSH |
| Q14872 | TAT | 57 | YQCTFEGCPRTYSTAGNLRTHQKTH |
| Q14872 | TGC | 58 | FRCDHDGCGKAFAASHHLKTHVRTH |
| ZIC3 | TAG | 59 | FPCPFPGCGKIFARSENLKIHKRTH |
| Z143 | CTT | 60 | FKCPFEGCGRSFTTSNIRKVHVRTH |
| Z143 | CGT | 61 | FRCEYDGCGKLYTTAHHLKVHERSH |
| O00153 | AAT | 62 | FMCHESGCGKQFTTAGNLKNHRRIH |
| Z143 | AAC | 63 | YYCTEPGCGRAFASATNYKNHVRIH |
| Q14872 | TCT | 64 | FVCNQEGCGKAFLTSHSLRIHVRVH |
| O00153 | TGT | 65 | FICPAEGCGKSFYVLQRLKVHMRTH |
| Q14872 | GCT | 66 | FNCESEGCSKYFTTLSDLRKHIRTH |
| Z143 | GCT | 67 | YRCSEDNCTKSFKTSGDLQKHIRTH |
| BTE1 | GCG | 68 | FPCTWPDCLKKFSRSDELTRHYRTH |
| O15391 | TAA | 69 | FVCPFDVCNRKFAQSTNLKTHILTH |
| Z143 | GNC | 70 | YVCTVPGCDKRFTEYSSLYKHHVVH |
| O43591 | GGT | 71 | HVCEHCNAAFRTNYHLQRHVFIH |
| BCL6 | TAG | 72 | YRCNICGAQFNRPANLKTHTRIH |
| O75626 | TAC | 73 | HECQVCHKRFSSTSNLKTHLRLH |
| O75626 | YAA | 74 | YECNVCAKTFGQLSNLKVHLRVH |
| BCL6 | NGA | 75 | YKCETCGARFVQVAHLRAHVLIH |
| O75626 | GGA | 76 | FKCQTCNKGFTQLAHLQKHYLVH |
| ZN45 | N | 77 | YRCDVCGKRFRQRSYLQAHQRVH |
| | (N/T)A | | |
| BCL6 | YTY | 78 | YPCEICGTRFRHLQTLKSHLRIH |
| GFI1 | GCA | 79 | YPCQYCGKRFHQKSDMKKHTFIH |
| Z263 | GAN | 80 | YQCNICGKCFSCNSNLHRHQRTH |
| ZN75 | TAY | 81 | YRCSWCGKSFSHNTNLHTHQRIH |
| Z186 | TTT | 82 | YKCIECGKTFTVNQLLTLHHRTH |
| | (YYY) | | |
| Z136 | TTT | 83 | FKCKQCGKAFSCSPTLRIHERTH |
| | (YYY) | | |
| Z136 | TGA | 84 | YKCKVCGKAFDYPSRFRTHERSH |
| Z136 | TTT | 85 | YKCKVCGKPFHSLSSFQVHERIH |
| | (YYY) | | |
| Z177 | TTA | 86 | YECKECGKAFRNSSCLRVHVRTH |
| Z136 | TNN | 87 | FECKRCGKAFRSSSSFRLHERTH |
| O60765 | A/ | 88 | YRCNECGKGFTSISRLNRHRIIH |
| | T—YT | | |
| ZN42 | TYT | 89 | YHCGECGLGFTQVSRLTEHQRIH |
| ZN42 | CGG | 90 | FVCGDCGQGFVRSARLEEHRRVH |
| O14913 | TCG | 91 | YKCEKCGKGFFRSSDLQHHQKIH |
| O14913 | C—O/ | 92 | YKCEECGKGFSRSSKLQEHQTIH |
| | T—G | | |
| ZN45 | YYC | 93 | YKCEECGKGFCRASNLLDHQRGH |
| ZN45 | AAA | 94 | YKCEECGKGFSQASNLLAHQRGH |
| ZN45 | NAG | 95 | YQCEECGKGFCRASNFLAHRGVH |
| Z239 | YYG | 96 | YKCEQCGKGFTRSSSLLIHQAVH |
| O94892 | YNY | 97 | YRCSECGKGFIVNSGLMLHQRTH |
| ZN45 | AAY | 98 | YQCAECGKGFSVGSQLQAHQRCH |
| ZN45 | NGY | 99 | YKCEECGKGFSVGSHLQAHQISH |
| ZN45 | YCG | 100 | YQCDACGKGFSRSSDFNIHFRVH |
| ZN45 | CCG | 101 | YKCGTCGKGFSRSSDLNVHCRIH |
| ZN45 | TGA | 102 | YKCNACGKSFSYSSHLNIHCRIH |
| Z239 | TCA | 103 | YQCYECGKGFSQSSDLRIHLRVH |
| Z239 | YAA | 104 | YKCGECGKGFSQSSNLHIHRCIH |
| Z239 | YGA | 105 | YKCDKCGKGFSQSSKLHIHQRVH |
| Z239 | CGA | 106 | YHCGKCGKGFSQSSKLLIHQRVH |
| O60765 | AYA | 107 | FKCSECGRAFSQSASLIQHERIH |
| O60792 | GYY | 108 | YECKECGKAFIRSSSLAKHERIH |
| ZN07 | ATA | 109 | YPCKECGKAFSQSSTLAQHQRMH |
| O43296 | AYY | 110 | YKCSECGKAFSRSSSLTQHQRMH |
| Z134 | ATG | 111 | YKCSECGKAFSRKDTLVQHQRIH |
| Z134 | ATG | 112 | YECSECGKAFSRKATLVQHQRIH |
| ZN84 | AYC | 113 | YECSECGKAFSEKLSLTNHQRIH |
| Z191 | AYG | 114 | YGCVECGKAFSRSSILVQHQRVH |
| ZN24 | ACG | 115 | YGCVECGKAFSRSSILVQHQRVH |
| O43338 | GTA | 116 | YVCGQCGKSFSQRATLIKHHRVH |
| O43339 | GTA | 117 | YECSQCGKSFSQKATLVKHQRVH |
| O43338 | AYA | 118 | YDCGQCGKSFIQKSSLIQHQVVH |
| O43339 | ANA | 119 | YECGQCGKSFSQKSGLIQHQVVH |
| O43338 | CAA | 120 | YECGECGKSFSQSSNLIEHCRIH |
| Q13398 | AAA | 121 | YECGECGKSFSQRSNLMQHRRVH |
| Z135 | CYA | 122 | YECGECGKAFSQSTLLTEHRRIH |
| Q13398 | ACA | 123 | YECSECGKSFSQSSSLIQHRRVH |
| O14709 | AAA | 124 | YKCNECGKAFSQSAYLLNHQRIH |
| O14709 | CAA | 125 | YKCNECGKVFSQNAYLIDHQRLH |
| O14709 | CAA | 126 | YKCTECGKAFTQSAYLFDNQRLH |
| O14709 | CAA | 127 | YKCDECGKTFAQTTYLIDHQRLH |
| O60792 | AAA | 128 | YNCNECRKTFSQSTYLIQHQRIH |
| O15535 | ANA | 129 | YHCKECGKVFSQSAGLIQHQRIH |
| Q15776 (a) | TNA | 130 | YHCKECGKAFSQNTGLILHQRIH |
| Q15776 (b) | TNA | 131 | YQCNQCGKAFSQSAGLILHQRIH |
| Q15776 | CNA | 132 | YKCNECGRAFSQKSGLIEHQRIH |
| ZN84 | AAC | 133 | YGCNECGRAFSEKSNLINHQRIH |
| Z191 | ANA | 134 | YKCLECGKAFSQNSGLINHQRIH |
| ZN24 | ANA | 135 | YKCLECGKAFSQNSGLINHQRIH |
| O60765 | AYA | 136 | YRCEECGISFGQSSALIQHRRIH |
| ZN07 | YYA | 137 | YRCEECGKAFGQSSSLIHHQRIH |
| O43340 | ACA | 138 | YECDECGKSYSQSSALLQHRRVH |
| Z135 | CYY | 139 | YKCQECGKAFSHSSALIEHHRTH |
| O43340 | AYA | 140 | YDCSECGKSFRQVSVLIQHQRVH |
| O43340 | AYA | 141 | YVCSECGKSFGQKSVLIQHQRVH |
| Q13398 | AYT | 142 | YQCSQCGKSFGCKSVLIQHQRVH |
| O15535 | GNA | 143 | HKCDECGKSFTQSSGLIRHQRIH |
| Q15776 | GNA | 144 | HKCDECGKSFAQSSGLVRHWRIH |
| O75802 | ANG | 145 | HKCEECGKAFSRSSGLIQHQRIH |
| Z189 | ANG | 146 | HKCEECGKAFSRSSGLIQHQRIH |
| O75802 | ANG | 147 | HKCDECGKAFSRNSGLIQHQRIH |
| Q13398 | YYG | 148 | HECNECGKSFSRSSSLIHHRRLH |
| Z195 | YAA | 149 | YKCDECGKNFTQSSNLIVHKRIH |
| O43309 | CYA | 150 | YKCDKCGKAFTQRSVLTEHQRIH |
| Z195 | CGA | 151 | YKCDECGKAYTQSSHLSEHRRIH |
| ZN45 | YYA | 152 | YKCERCGKAFSQFSSLQVHQRVH |
| O60893 | YYN | 153 | YECEDCGKTFIGSSALVIHQRVH |
| ZN07 | TAT | 154 | YECLQCGKAFSMSTQLTIHQRVH |
| O60893 | CYA | 155 | YECDDCGKTFSQSCSLLEHHKIH |
| Q15776 | NGG | 156 | YECDECGKTFRRSSHLIGHQRSH |
| ZN84 | YGG | 157 | YECGECGKAFSRKSHLISHWRTH |
| Z177 | YGA | 158 | YECDHCGKSFSQSSHLNVHKRTH |
| O43296 | AYG | 159 | YECMECGKAFNRKSYLTQHQRIH |
| O43296 | GNG | 160 | YECVECGKAFTRMSGLTRHKRIH |
| O43340 | AGG | 161 | YECRECGKSFTRKNHLIQHKTVH |
| Z134 | AAG | 162 | YECSECGKTFSRKDNLTQHKRIH |
| O43338 | CGA | 163 | YECSECGKSFSQTSHLNDHRRIH |
| O75467 | AGA | 164 | YECAQCGKAFSQTSHLTQHQRIH |
| Z135 | AGA | 165 | YECSECGKAFRQSIHLTQHLRIH |
| Z135 | AGA | 166 | YECHDCGKSFRQSTHLTQHRRIH |
| Z205 | AGG | 167 | YACTDCGKRFGRSSHLIQHQIIH |
| O43296 | AGG | 168 | YECTECGKTFIKSTHLLQHHMIH |
| O75290 | AAG | 169 | YECKECGKYFSRSANLIQHQSIH |
| O75290 | AGG | 170 | YECKECGKGFNRGAHLIQHQKIH |
| O75290 | AGG | 171 | YECKECGKGFNRGAHLIQHQKIH |
| O60792 | CGA | 172 | YTCNECGKAFSQRGHFMEHQKIH |
| O75123 | CGA | 173 | YTCDQCGKGFGQSSHLMEHQRIH |
| O43337 | GYA | 174 | YECNACGKAFSQSSTLIRHYLIH |
| O75802 | GYY | 175 | YECNYCGKTFSVSSTLIRHQRIH |
| Z165 | GGY | 176 | YECSECGKTFRVSSHLIRHFRIH |
| Z124 | CYY | 177 | YVCNNCGKGFRCSSSLRDHERTH |
| Z135 | AYY | 178 | YGCNECGKTFSHSSSLSQHERTH |
| O15361 | GAY | 179 | YDCNHCGKSFNHKTNLNKHERIH |
| O75123 | AAA | 180 | YVCNECGKRFSQTSNFTQHQRIH |
| Q13398 | AAY | 181 | YVCGECGKSFSHSSNLKNHQRVH |
| ZN35 | YYA | 182 | YTCNECGKAFRQRSSLTVHQRTH |
| Z157 | YYC | 183 | YECTECGKTFSEKATLTIHQRTH |
| O43338 | GYY | 184 | YECDECGKAFGSKSTLVRHQRTH |
| ZN84 | TYC | 185 | YECSECGKAFGEKSSLATHQRTH |
| ZN07 | GAA | 186 | YGCRECGKAFSQQSQLVRHQRTH |
| ZN84 | YAA | 187 | YNCSQCGKAFSQKSQLTSHQRTH |
| Z186 | YGY | 188 | YACDHCEKAFSHKSKLTVHQRTH |
| O43338 | GGC | 189 | YVCGECGKAFMFKSKLVRHQRTH |
| OZF | YYA | 190 | YECNVCGKAFSQSSSLTVHVRSH |
| O95779 | YYY | 191 | YKCKECGKAFNHCSLLTIHERTH |
| Z135 | GYY | 192 | YACRDCGKAFTHSSSLTKHQRTH |
| ZN80 | GYA | 193 | YECKECGKGFYYSYSLTRHTRSH |
| Z177 | GYC | 194 | YECSDCGKAFIDQSSLKKHTRSH |
| Z177 | GYY | 195 | YDCKECGKAFTVPSSLQKHVRTH |
| O43337 | ACT | 196 | YDCMACGKAFRCSSELIQHQRIH |
| Q14585 | AGY | 197 | YECKECEKAFRSGSKLIQHQRMH |
| Q14585 | AAY | 198 | YECIDCGKAFGSGSNLTQHRRIH |
| Q14585 | GYY | 199 | YECKACGMAFSSGSALTRHQRIH |
| Q14585 | AYY | 200 | YECKECGKAFYSGSSLTQHQRIH |
| Q14585 | AAY | 201 | YECKECGKAFGSGANLAYHQRIH |
| Q14585 | GAY | 202 | FECKECGKAFGSGSNLTHHQRIH |
| Q14585 | ACY | 203 | YVCKECGKAFNSGSDLTQHQRIH |
| O60792 | ACY | 204 | YQCHECGKTFSYGSSLIQHRKIH |
| O60893 | GNA | 205 | HYCHECGKSFAQSSGLTKHRRIH |
| Z165 | GCC | 206 | YECNECGKSFAESSDLTRHRRIH |
| O60893 | GAY | 207 | YECEECGKVFSHSSNLIKHQRTH |
| Q15776 | NGY | 208 | YECNECGKAFSHSSHLIGHQRIH |
| Z135 | GYY | 209 | YQCGECGKAFSHSSSLTKHQRIH |
| Z165 | GGY | 210 | HQCNECGKAFRHSSKLARHQRIH |
| Z135 | TYG | 211 | YECHECLKGFRNSSALTKHQRIH |
| O43361 | YGC | 212 | YECNECGKFFLDSYKLVIHQRIH |
| O43361 | YGC | 213 | YECSECGKFFRDSYKLIIHQRVH |
| Z140 | YYG | 214 | YGCHECGKTFGRRFSLVLHQRTH |
| O60792 | AAA | 215 | YECNECGKAFSQHSNLTQHQKTH |
| Z135 | ANA | 216 | YKCTQCGRTFNQIAPLIQHQRTH |
| Z135 | ANA | 217 | YECNQCGRAFSQLAPLIQHQRIH |
| Z135 | ANA | 218 | YECHECGKAFTQITPLIQHQRTH |
| O43309 | AGA | 219 | YKCNECGKAFGRWSALNQHQRLH |
| ZN83 | AGA | 220 | YKCNECGKVFHNMSHLAQHRRIH |
| ZN83 | AGY | 221 | YRCNVCGKVFHHISHLAQHQRIH |
| ZN83 | AGA | 222 | YKCNECGKVFNQISHLAQHQRIH |
| O14709 | CAY | 223 | FECSECGRAFSSNRNLIEHKRIH |
| ZN74 | GYA | 224 | YKCSECGRAFSQNHCLIKHQKIH |
| Q13398 | ANA | 225 | YECSECGKSFSQNFSLIYHQRVH |
| O75123 | GYA | 226 | FECKECGKGFSQSSLLIRHQRIH |
| Z132 (a) | GGA | 227 | FECSECGRDFSQSSHLLRHQKVH |
| Z132 | GYA | 228 | YECNECGKFFSQNSILIKHQKVH |
| Z132 (b) | GGA | 229 | YECDECGKAFSNRSHLIRHEKVH |
| Z132 | GGN | 230 | YECSECGRAFSSNSHLVRHQRVH |
| Z132 | AAA | 231 | YECSECGRAFNNNSNLAQHQKVH |
| Z134 | ATY | 232 | YKCSDCGKVFRHKSTLVQHESIH |
| O75290 | AAT | 233 | YECKECGKAFRLYLQLSQHQKTH |
| Z157 | AYC | 234 | YECGECGKNFRAKKSLNQHQRIH |
| Z157 | TTT | 235 | YECGECGKFFRMKMTLNNHQRTH |
| ZN07 | AAT | 236 | YECAECGKVFRLCSQLNQHQRIH |
| Z157 | AYT | 237 | YECSECGKIFSMKKSLCQHRRTH |
| O43361 | GGY | 238 | YECNKCGKFFMYNSKLIRHQKVH |
| O43361 | GTY | 239 | YKCSKCGKFFRYRCTLSRHQKVH |
| Z157 | CGY | 240 | YECNECGNAFYVKARLIEHQRMH |
| Z157 | CGY | 241 | YECSECGNAFYVKVRLIEHQRIH |
| O75123 | AGG | 242 | FECNECGKAFIRSSKLIQHQRIH |
| ZN07 | AGT | 243 | FKCTECGKAFRLSSKLIQHQRIH |
| O75123 | GYT | 244 | YECNECGKAFFLSSYLIRHQKIH |
| O75802 | AAT | 245 | HKCGECGKAFRLSTYLIQHQKIH |
| Z174 | GCG | 246 | YKCDDCGKSFTWNSELKRHKRVH |
| | RNA | | |
| Z202 | GCG | 247 | YRCDDCGKHFRWTSDLVRHQRTH |
| | RNA | | |
| O43345 | GTG | 248 | YKCEECGKAYKWPSTLSYHKKIH |
| | RNA | | |
| O43345 | CA? | 249 | YKCEECGKAFNWSSNLMEHKKIH |
| | RNA | | |
| O75346 | TAA | 250 | YRCEECGKAFNQSANLTTHKRIH |
| ZN43 | TAA | 251 | YKCEECGKAFTQSSNLTTHKKIH |
| ZN85 | GGA | 252 | YKCEECGKAFNQSSKLTKHKKIH |
| ZN85 | GAA | 253 | YTCEECGKAFNQSSNLTKHKRIH |
| Q02313 | GAA | 254 | YKCEECGKAFNQLSNLTRHKVIH |
| Q02313 | CAA | 255 | YKCEECGKAFKQFSNLTDHKKIH |
| Z141 | GTG | 256 | YKCEECGKAFNRSTTLTKHKRIH |
| ZN91 | TTG | 257 | YKCEECGKAFSRSSTLTKHKTIH |

### Example 2

### List of All Human C₂H₂ Zinc Fingers

This list represents an even more comprehensive database of human zinc fingers, including those with non-DNA-binding activities such as those mediating protein-protein interactions and those involved in RNA binding. By including fingers from this database into a natural finger selection system as disclosed herein, many new zinc finger proteins having unique target specificities can be obtained. All of these peptides would necessarily possess properties required for potential therapeutic agents, such as non-immunogenicity.

The fingers listed below are in a format that can be linked with classical canonical “TGEKP” linkers (i.e . . . TGEKP—zinc finger peptide sequence—TGEKP— zinc finger peptide sequence -TGEKP—etc . . . ). For each peptide sequence, an oligonucleotide is designed to encode the peptide sequence; the oligonucleotide can then be linked into a library selection system, as described in the Examples infra.

Human zinc finger database
968 finger units

| | SEQ | |
|---|---|---|
| Name | ID NO | Peptide sequence |
| Q92981_HUMAN | 258 | HQCAHCEKTFNRKDHLKNFQTH |
| O76019_HUMAN | 259 | HQCAHCEKTFNRKDHLKNHLQTH |
| ZFY_HUMAN | 260 | HRCEYCKKGFRRPSEKNQHIMRH |
| ZFX_HUMAN | 261 | HRCEYCKKGFRRPSEKNQHIMRH |
| ZFX_BOVIN | 262 | HRCEYCKKGFRRPSEKNQHIMRH |
| Q15558_HUMAN | 263 | HRCEYCKKGFRRPSEKNQHIMRH |
| ZFX_HUMAN | 264 | HKCDMCDKGFHRPSELKKHVAAH |
| ZFY_HUMAN | 265 | HKCEMCEKGFHRPSELKKHVAVH |
| Q15558_HUMAN | 266 | HKCEMCEKGFHRPSELKKHVAVH |
| Z161_HUMAN | 267 | YTCSVCGKGFSRPDHLSCHVKHVH |
| MAZ_HUMAN | 268 | YNCSHCGKSFSRPDHLNSHVRQVH |
| O43829_HUMAN | 269 | YSCEVCGKSFIRAPDLKKHERVH |
| O00403_HUMAN | 270 | YSCEVCGKSFIRAPDLKKHERVH |
| Z151_HUMAN | 271 | HKCPHCDKKFNQVGNLKAHLKIH |
| Q92618_HUMAN | 272 | YKCPYCDHRASQKGNLKIHIRSH |
| ZFX_HUMAN | 273 | FRCKRCRKGFRQQSELKKHMKTH |
| Q14526_HUMAN | 274 | YPCTICGKKFTQRGTMTRHMRSH |
| HKR3_HUMAN | 275 | FECTECGYKFTRQAHLRRHMEIH |
| Q14526_HUMAN | 276 | YACDACGMRFTRQYRLTEHMRIH |
| O75626_HUMAN | 277 | YECNVCAKTFGQLSNLKVHLRVH |
| CTCF_HUMAN | 278 | HKCPDCDMAFVTSGELVRHRRYKH |
| O75701_HUMAN | 279 | YSCPDCSLRFAYTSLLAIHRRIH |
| O75701_HUMAN | 280 | YACSDCKSRFTYPYLLAIHQRKH |
| O43167_HUMAN | 281 | YACKDCGKVFKYNHFLAIHQRSH |
| O75850_HUMAN | 282 | CACPDCGRSFTQRAHMLLHQRSH |
| O75850_HUMAN | 283 | YACPDCGRGFSHGQHLARHPRVH |
| ZN42_HUMAN | 284 | FVCGDCGQGFVRSARLEEHRRVH |
| O75467_HUMAN | 285 | FRCVDCGKAFAKGAVLLSHRRIH |
| O15015_HUMAN | 286 | YKCSECGRAYRHRGSLVNHRHSH |
| O75701_HUMAN | 287 | YPCPDCGRRFRQRGSLAIHRRAH |
| Q92951_HUMAN | 288 | YECAICQRSFRNQSNLAVHRRVH |
| BCL6_HUMAN | 289 | YKCDRCQASFRYKGNLASHKTVH |
| ZN42_HUMAN | 290 | YACQDCGRRFHQSTKLIQHQRVH |
| O75701_HUMAN | 291 | YPCPDCGRRFTYSSLLLSHRRIH |
| O75701_HUMAN | 292 | HVCTDCGRRFTYPSLLVSHRRMH |
| O75701_HUMAN | 293 | HSCPDCGRNFSYPSLLASHQRVH |
| ZN42_HUMAN | 294 | YACVECGERFGRRSVLLQHRRVH |
| O43298_HUMAN | 295 | YGCGVCGKKFKMKHHLVGHMKIH |
| O15209_HUMAN | 296 | YDCPVCNKKFKMKHHLTEHMKTH |
| O43829_HUMAN | 297 | YACHMCDKAFKHKSHLKDHERRH |
| O00403_HUMAN | 298 | YACHMCDKAFKHKSHLKDHERRH |
| O60315_HUMAN | 299 | HQCQICKKAFKHKHHLIEHSRLH |
| Q12924_HUMAN | 300 | HECGICKKAFKHKHHLIEHMRLH |
| NIL2_HUMAN | 301 | HECGICKKAFKHKHHLIEHMRLH |
| Q12924_HUMAN | 302 | FKCTECGKAFKYKHHLKEHLRIH |
| O60315_HUMAN | 303 | FKCTECGKAFKYKHHLKEHLRIH |
| NIL2_HUMAN | 304 | FKCTECGKAFKYKHHLKEHLRIH |
| O95780_HUMAN | 305 | YKCEECGKAFKRCSHLNEHKRVQ |
| O95779_HUMAN | 306 | YKCEECGKAFKRCSHLNEHKRVQ |
| O43296_HUMAN | 307 | FKCSECGKVFNKKHLLAGHEKIH |
| O14709_HUMAN | 308 | YKCKECGKGFYRHSGLIIHLRRH |
| O14709_HUMAN | 309 | HKCKECGKGFIQRSSLLMHLRNH |
| ZN80_HUMAN | 310 | CKCVECGKVFNRRSHLLCYRQIH |
| O43337_HUMAN | 311 | YKCIECGKAFKRRSHLLQHQRVH |
| O60765_HUMAN | 312 | YICKECGKAFTLSTSLYKHLRTH |
| Z136_HUMAN | 313 | FECKRCGKAFRSSSSFRLHERTH |
| Z136_HUMAN | 314 | FVCKQCGKAFRSASTFQIHERTH |
| Z136_HUMAN | 315 | YVCKHCGKAFVSSTSIRIHERTH |
| Z136_HUMAN | 316 | FKCKQCGKAFSCSPTLRIHERTH |
| Z124_HUMAN | 317 | YVCNNCGKGFRCSSSLRDHERTH |
| Z177_HUMAN | 318 | YECKECGKAFRNSSCLRVHVRTH |
| Z124_HUMAN | 319 | YECKHCGKAFRYSNCLHYHERTH |
| O95780_HUMAN | 320 | YKCKECGKAFNHCSLLTIHERTH |
| O95779_HUMAN | 321 | YKCKECGKAFNHCSLLTIHERTH |
| Z124_HUMAN | 322 | YPCKQCGKAFRYASSLQKHEKTH |
| Z136_HUMAN | 323 | YECKQCGKAFSYLNSFRTHEMIH |
| Z136_HUMAN | 324 | YECKQCGKAFSYLPSLRLHERIH |
| O15060_HUMAN | 325 | YSCKVCGKRFAHTSEFNYHRRIH |
| Z136_HUMAN | 326 | YKCKVCGKPFHSLSPFRIHERTH |
| Z136_HUMAN | 327 | YKCKVCGKPFHSLSSFQVHERIH |
| Z136_HUMAN | 328 | YKCKVCGKAFDYPSRFRTHERSH |
| ZN35_HUMAN | 329 | YVCNECGKAFTCSSYLLIHQRIH |
| O15322_HUMAN | 330 | YNCKECGKSFRWSSYLLIHQRIH |
| Q92951_HUMAN | 331 | YRCDQCGKAFSQKGSLIVHIRVH |
| Q92951_HUMAN | 332 | YQCKECGKSFSQRGSLAVHERLH |
| Q92951_HUMAN | 333 | YECQECGKSFRQKGSLTLHERIH |
| OZF_HUMAN | 334 | YECNECGKAFSQRTSLIVHVRIH |
| OZF_HUMAN | 335 | YECNVCGKAFSQSSSLTVHVRSH |
| ZN07_HUMAN | 336 | YVCNDCGKAFSQSSSLIYHQRIH |
| Z151_HUMAN | 337 | CQCVMCGKAFTQASSLIAHVRQH |
| Z177 HUMAN | 338 | YDCKECGKAFTVPSSLQKHVRTH |
| OZF_HUMAN | 339 | FECKDCGKAFIQKSNLIRHQRTH |
| Z177_HUMAN | 340 | YECSDCGKAFIDQSSLKKHTRSH |
| Z177_HUMAN | 341 | YECSDCGKAFIFQSSLKKHMRSH |
| O60792_HUMAN | 342 | YECKECGKAFIRSSSLAKHERIH |
| Z161_HUMAN | 343 | YACTYCSKAFRDSYHLRRHESCH |
| Z161_HUMAN | 344 | HACEMCGKAFRDVYHLNRHKLSH |
| MAZ_HUMAN | 345 | HACEMCGKAFRDVYHLNRHKLSH |
| O60792_HUMAN | 346 | FKCDECDKTFTRSTHLTQHQKIH |
| O60792_HUMAN | 347 | YKCNECDKAFSRSTHLTEHQNTH |
| Z263_HUMAN | 348 | YKCNECGKSFRQGMHLTRHQRTH |
| Z263_HUMAN | 349 | HKCLECGKCFSQNTHLTRHQRTH |
| Z135_HUMAN | 350 | YECSQCGKAFRQSTHLTQHQRIH |
| Z135_HUMAN | 351 | YECHDCGKSFRQSTHLTQHRRIH |
| Z135_HUMAN | 352 | YECSECGKAFRQSIHLTQHLRIH |
| O75467_HUMAN | 353 | YECAQCGKAFSQTSHLTQHQRIH |
| ZN07_HUMAN | 354 | YECLQCGKAFSMSTQLTIHQRVH |
| O95270_HUMAN | 355 | YPCQFCGKRFHQKSDMKKHTYIH |
| GFI1_HUMAN | 356 | YPCQYCGKRFHQKSDMKKHTFIH |
| O75850_HUMAN | 357 | FPCTECEKRFRKKTHLIRHQRIH |
| Q15552_HUMAN | 358 | FRCDECGMRSIQKYHMERHKRTH |
| O43591_HUMAN | 359 | FRCDECGMRFIQKYHMERHKRTH |
| Q15552_HUMAN | 360 | FQCSQCDMRFIQKYLLQRHEKIH |
| O43591_HUMAN | 361 | FQCSQCDMRFIQKYLLQRHEKIH |
| O75850 HUMAN | 362 | FPCSECDKRFSKKAHLTRHLRTH |
| O75850_HUMAN | 363 | YPCAECGKRFSQKIHLGSHQKTH |
| O94892_HUMAN | 364 | FMCSECGKGFTMKRYLIVHQQIH |
| O43336_HUMAN | 365 | YQCSECGKSFIYKQSLLDHHRIH |
| O43167_HUMAN | 366 | FKCNECGKGFAQKHSLQVHTRMH |
| O43167_HUMAN | 367 | YTCDQCGKYFSQNRQLKSHYRVH |
| PLZF_HUMAN | 368 | YECNGCDKKFSLKHQLETHYRVH |
| HKR3_HUMAN | 369 | YACPTCHKKFLSKYYLKVHNRKH |
| O43336_HUMAN | 370 | YVCNVCGKSFRHKQTFVGHQQRIH |
| O43336_HUMAN | 371 | YVCNICGKSFLHKQTLVGHQQRIH |
| Z134_HUMAN | 372 | YDCSDCGKSFGHKYTLIKHQRIH |
| Z200_HUMAN | 373 | YDCNHCGKSFNHKTNLNKHERIH |
| O15361_HUMAN | 374 | YDCNHCGKSFNHKTNLNKHERIH |
| ZN84_HUMAN | 375 | YDCNHCGKAFSRKSQLVRHQRTH |
| ZN84_HUMAN | 376 | FECRECGKAFSRKSQLVTHHRTH |
| ZN07_HUMAN | 377 | YGCRECGKAFSQQSQLVRHQRTH |
| ZN84_HUMAN | 378 | YRCIECGKAFSQKSQLINHQRTH |
| ZN84_HUMAN | 379 | YGCSECRKAFSQKSQLVNHQRIH |
| ZN84_HUMAN | 380 | HGCIQCGKAFSQKSHLISHQMTH |
| ZN84_HUMAN | 381 | YNCSQCGKAFSQKSQLTSHQRTH |
| ZN84_HUMAN | 382 | YVCSECGKAFCQKSHLISHQRTH |
| Z157_HUMAN | 383 | FECNECGKSFGRKSQLILHTRTH |
| ZN84_HUMAN | 384 | FECSECGKAFSRKSHLIPHQRTH |
| ZN84_HUMAN | 385 | YECGECGKAFSRKSHLISHWRTH |
| Z136_HUMAN | 386 | YHCKECGKAYSCRASFQRHMLTH |
| Z136_HUMAN | 387 | YECKECGEAFSCIPSMRRHMIKH |
| Z136_HUMAN | 388 | YECQECGKAFTCITSVRRHMIKH |
| ZN80_HUMAN | 389 | YECQECGKAFPEKVDFVRHMRIH |
| O43338_HUMAN | 390 | YVCGECGKAFMFKSKLVRHQRTH |
| O43338_HUMAN | 391 | YECDECGKAFGSKSTLVRHQRTH |
| Z133_HUMAN | 392 | YACGECGRGFSQKSNLVAHQRTH |
| Z133_HUMAN | 393 | YMCSECGRGFSQKSNLIIHQRTH |
| Z133_HUMAN | 394 | YACKDCGRGFSQQSNLIRHQRTH |
| Z133_HUMAN | 395 | YACSDCGLGFSDRSNLISHQRTH |
| Z133_HUMAN | 396 | YACRECGRGFNRKSTLIIHERTH |
| Z133_HUMAN | 397 | YVCRECGRGFSHQAGLIRHKRKH |
| Z133_HUMAN | 398 | CVCRECGQGFLQKSHLTLHQMTH |
| Z133_HUMAN | 399 | YVCRECGKGFSQKSAVVRHQRTH |
| O94892_HUMAN | 400 | YICSECGKGFPRKSNLIVHQRNH |
| O94892_HUMAN | 401 | YICNECGKGFPGKRNLIVHQRNH |
| O94892_HUMAN | 402 | YTCSECGKGFPLKSRLTVHQRTH |
| O94892_HUMAN | 403 | YICSECGKGFTTKHYVIIHQRNH |
| O94892_HUMAN | 404 | YICSECGKGFTGKSMLIIHQRTH |
| O94892_HUMAN | 405 | YLCSECGKGFTVKSMLIIHQRTH |
| O94892_HUMAN | 406 | YGCNECGKGFTMKSRLIVHQRTH |
| O94892_HUMAN | 407 | YICNECGKGFTMKSRMIEHQRTH |
| O94892_HUMAN | 408 | FICSECGKVFTMKSRLIEHQRTH |
| O94892_HUMAN | 409 | YICNECGKGFAFKSNLVVHQRTH |
| Z186_HUMAN | 410 | YECNECGKTFHQKSFLTVHQRTH |
| Z186_HUMAN | 411 | YECNELGKTFHCKSFLTVHQKTH |
| Z186_HUMAN | 412 | YGCNECGKTVRCKSFLTLHQRTH |
| ZN35_HUMAN | 413 | YTCNECGKAFRQRSSLTVHQRTH |
| Z186_HUMAN | 414 | YQCSECGKTFSQKSYLTIHHRTH |
| Z157_HUMAN | 415 | YECSECGKTFRVKISLTQHHRTH |
| Z186_HUMAN | 416 | YKCIECGKTFTVNQLLTLHHRTH |
| Z157_HUMAN | 417 | YECTECGKTFSEKATLTIHQRTH |
| ZN84_HUMAN | 418 | YACSDCRKAFFEKSELIRHQTIH |
| ZN84_HUMAN | 419 | YECSLCRKAFFEKSELIRHLRTH |
| Z140_HUMAN | 420 | YECNECRKALRCHSFLIKHQRIH |
| ZN84_HUMAN | 421 | YECNECRKAFREKSSLINHQRIH |
| ZN84_HUMAN | 422 | YECSECRKAFRERSSLINHQRTH |
| ZN84_HUMAN | 423 | YECSECGKAFGEKSSLATHQRTH |
| ZN84_HUMAN | 424 | YECSECGKAFSEKLSLTNHQRIH |
| O43339_HUMAN | 425 | YECSKCGKAFRGKYSLVQHQRVH |
| Z157_HUMAN | 426 | YECSECGKIFSMKKSLCQHRRTH |
| Z157_HUMAN | 427 | YECGECGKFFRMKMTLNNHQRTH |
| Z157_HUMAN | 428 | YECGECGKNFRAKKSLNQHQRIH |
| O43361_HUMAN | 429 | YKCSECGKAFSLKHNVVQHLKIH |
| Z134_HUMAN | 430 | YECSECGKAFSRKATLVQHQRIH |
| Z134_HUMAN | 431 | YKCSECGKAFSRKDTLVQHQRIH |
| Z134_HUMAN | 432 | YECSECGKTFSRKDNLTQHKRIH |
| O14709_HUMAN | 433 | YKCKECGKVFIRSKSLLLHQRVH |
| O14709 HUMAN | 434 | YECDECGKCFILKKSLIGHQRIH |
| O14709_HUMAN | 435 | YECNECGKVFILKKSLILHQRFH |
| O14709_HUMAN | 436 | YKCNKCQKAFILKKSLILHQRIH |
| Z140_HUMAN | 437 | YACAECDKAFSRSFSLILHQRTH |
| Z140_HUMAN | 438 | YGCHECGKTFGRRFSLVLHQRTH |
| O95878_HUMAN | 439 | YACAQCGKTFNNTSNLRTHQRIH |
| O14709_HUMAN | 440 | YKCDMCCKHFNKISHLINHRRIH |
| ZN83_HUMAN | 441 | FKCDICGKIFNKKSNLASHQRIH |
| ZN07_HUMAN | 442 | HQCEDCEKIFRWRSHLIIHQRIH |
| Z137_HUMAN | 443 | HKCDDCGKVLTSRSHLIRHQRIH |
| Z140_HUMAN | 444 | HECKDCNKTFSYLSFLIEHQRTH |
| Z189_HUMAN | 445 | HKCSDCGKAFSWKSHLIEHQRTH |
| O75802_HUMAN | 446 | HKCSDCGKAFSWKSHLIEHQRTH |
| O14709_HUMAN | 447 | YKCNDCGKVFSYRSNLIAHQRIH |
| O43309_HUMAN | 448 | YGCDDCGKAFSQHSHLIEHQRIH |
| O75123_HUMAN | 449 | YTCDQCGKGFGQSSHLMEHQRIH |
| O43336_HUMAN | 450 | YNCTACEKAFIYKNKLVEHQRIH |
| O43309_HUMAN | 451 | YKCDVCEKAFIQRTSLTEHQRIH |
| O60792_HUMAN | 452 | YKCDQCGKGFIEGPSLTQHQRIH |
| O43309_HUMAN | 453 | YKCDKCGKAFTQRSVLTEHQRIH |
| ZN91_HUMAN | 454 | YKCEECGKAFKQLSTLTTHKRIH |
| ZN91_HUMAN | 455 | YKCKECGKAFKQFSTLTTHKIIH |
| ZN91_HUMAN | 456 | YKCKECDKTFKRLSTLTKHKIIH |
| ZN91_HUMAN | 457 | YKCKECDKTFKRLSTLTKHKIIH |
| ZN85_HUMAN | 458 | YKCEKCGKAFNHFSHLTTHKIIH |
| ZN85_HUMAN | 459 | YKCEECGKAFNRFSTLTTHKIIH |
| ZN43_HUMAN | 460 | YKCEECGKAFNQFSTLTKHKIIH |
| ZN43_HUMAN | 461 | YTCEECGKVFNWSSRLTTHKRIH |
| ZN43_HUMAN | 462 | YKCEECGKAFNKSSILTTHKIIR |
| O75437_HUMAN | 463 | YKWEKFGKAFNRSSHLTTDKITH |
| O43345_HUMAN | 464 | YKCEEGGKAFNWSSTLTYYKSAH |
| ZN91_HUMAN | 465 | YKCEECGKAFNQSSNLTTHKIIH |
| ZN91_HUMAN | 467 | YKCEECGKAFNRSSKLTTHKIIH |
| Q02313_HUMAN | 468 | YKCEECGKAFNQSSTLTTHNIIH |
| ZN91_HUMAN | 469 | YKCEECGKAFNHSSSLSTHKIIH |
| ZN43_HUMAN | 470 | YKCEECGKAFKLSSTLSTHKIIH |
| ZN91_HUMAN | 471 | YKCEECGKAFSQSSTLTTHKIIH |
| Q02313_HUMAN | 472 | YKCEECGKAFNQSSTLTTHKRIH |
| O95780_HUMAN | 473 | YKCEECGKAFNSSSILTEHKVIH |
| O95779_HUMAN | 474 | YKCEECGKAFNSSSILTEHKVIH |
| ZN91_HUMAN | 475 | YKCKECGKAFKHSSALAKHKIIH |
| ZN85_HUMAN | 476 | YKCKECGKAFKHSSTLTKHKIIH |
| ZN85_HUMAN | 477 | YKCEECDKAFKWSSVLTKHKIIH |
| ZN43_HUMAN | 478 | YKCEECGKAFKWSSTLTKHKIIH |
| ZN85_HUMAN | 479 | YKCEECGKGFKWPSTLTIHKIIH |
| ZN91_HUMAN | 480 | YKCGECGKAFKESSALTKHKIIH |
| ZN91_HUMAN | 481 | YKCEECGKAFRKSSTLTEHKIIH |
| ZN91_HUMAN | 482 | YKCEECGKAFRQSSTLTKHKIIH |
| Q02313 HUMAN | 483 | YKCGECGKAFNQSSALNTHKIIH |
| ZN91_HUMAN | 484 | CKCKECEKTFHWSSTLTNHKEIH |
| O75437_HUMAN | 485 | YKCKECGKTFNWSSTLTNHRKIY |
| ZN91_HUMAN | 486 | YKCKECGKAFSNSSTLANHKITH |
| ZN91_HUMAN | 487 | YKCKECGKAFSNSSTLANHKITH |
| O43345_HUMAN | 488 | YKCKECGKTFIKVSTLTTHKAIH |
| O43345_HUMAN | 489 | YKCEECGKTFSKVSTLTTHKAIH |
| O43345_HUMAN | 490 | YKCEECGKTFSKVSTLTTHKAIH |
| O43345_HUMAN | 491 | YKCEECGKAFSKVSTLTTHKAIH |
| O43345_HUMAN | 492 | YKCKECGKAFSKVSTLITHKAIH |
| O95270_HUMAN | 493 | YACRMCGKAFKRSSTLSTHLLIH |
| GFI1_HUMAN | 494 | YDCKICGKSFKRSSTLSTHLLIH |
| O75346 HUMAN | 495 | YKCIICGKAFKRSSTLTTHKKIH |
| ZN43_HUMAN | 496 | YKCKECGKAFNQYSNLTTHNKIH |
| ZN85_HUMAN | 497 | YKCKECGKAFNRSSTLTTHRKIH |
| ZN91_HUMAN | 498 | YKCSEECDKAFIWSSTLTEHKRIH |
| ZN91_HUMAN | 499 | YKCEECGKAFISSSTLNGHKRIH |
| ZN43_HUMAN | 500 | YKCEECGKAFNYSSHLNTHKRIH |
| O95780 HUMAN | 501 | YKCEECGKAFNWSSILTEHKRIH |
| O95779_HUMAN | 502 | YKCEECGKAFNWSSILTEHKRIH |
| O43345_HUMAN | 503 | YKCEECGKAFNWSSNLMEHKRIH |
| O43345_HUMAN | 504 | YKCEECGKAFNWSSNLMEHKRIH |
| O43345_HUMAN | 505 | YKCEECGKAFNWSSNLMEHKKIH |
| O43345_HUMAN | 506 | YKCEECGKAFNWSSNLMEHKKIH |
| ZN91_HUMAN | 507 | FKCKECGKAFIWSSTLTRHKRIH |
| ZN91_HUMAN | 508 | FKCKECGKGFIWSSTLTRHKRIH |
| ZN91_HUMAN | 509 | YKCEECGKAFLWSSTLRRHKRIH |
| ZN91_HUMAN | 510 | YKCEECGKAFLWSSTLTRHKRIH |
| Q02313_HUMAN | 511 | YKCEAYGRAFNWSSTLNKHKRIH |
| ZN91_HUMAN | 512 | YKFEECGKAFRQSLTLNKHKIIH |
| Z141_HUMAN | 513 | YKCEECGKAFRRSTDRSQHKKIH |
| O75346_HUMAN | 514 | YKCEECGKAFNWSSDLNKHKKIH |
| ZN91_HUMAN | 515 | YKCEECGKAFNWSSSLTKHKRIH |
| ZN91_HUMAN | 516 | YKCEECGKAFNWSSSLTKHKRFH |
| ZN85_HUMAN | 517 | YKCEECGKAFNWSSTLTKHKRIH |
| ZN43_HUMAN | 518 | YKCEECGKAPNWPSTLTKHNRIH |
| ZN43_HUMAN | 519 | YKCEECGKAFNWPSTLTKHKRIH |
| O75437_HUMAN | 520 | YKCEECGKAFFWSSTLTKHKRIH |
| O95780_HUMAN | 521 | YKCEECGKAFNWCSSLTKHKRIH |
| O95779_HUMAN | 522 | YKCEECGKAFNWCSSLTKHKRIH |
| ZN43_HUMAN | 523 | YKCEECGKAFSRSSNLTKHKKIH |
| ZN43_HUMAN | 524 | YKCTECGEAFSRSSNLTKHKKIH |
| ZN91_HUMAN | 525 | YKCEECGKAFSRSSTLTKHKTIH |
| O75437_HUMAN | 526 | YKCEECGKAFNRSSTFTKHKVIH |
| Z141_HUMAN | 527 | YKCEECGKAFNRFTTLTKHKRIH |
| Z141_HUMAN | 528 | YKCEECGKAFNRSTTLTKHKRIH |
| ZN43_HUMAN | 529 | CKCEKCGKAFNCPSIITKHKRIN |
| O43345_HUMAN | 530 | YKCEACGKAYNTFSILTKHKVIH |
| O43345_HUMAN | 531 | YKCEECGKAFSTFSILTKHKVIH |
| O43345_HUMAN | 532 | YKCEECGKSFSTFSILTKHKVIH |
| O43345_HUMAN | 533 | YKCEECGKSFSTFSVLTKHKVIH |
| O43345_HUMAN | 534 | YKCEECGKGFVMFSILAKHKVIH |
| O43345_HUMAN | 535 | YKCEECGKGFSMFSILTKHEVIH |
| O43345_HUMAN | 536 | YKCEECGKGFSMFSILTKHEVIH |
| O43345_HUMAN | 537 | YKCKECGKAFSKFSILTKHKVIH |
| O43345_HUMAN | 538 | YKCKECGKAFSKFSILTKHKVIH |
| O43345_HUMAN | 539 | YKCKECGKAFSKFSILTKHKVIH |
| O43345_HUMAN | 540 | YRCKECGKAFSKFSILTKHKVIH |
| Z195_HUMAN | 541 | FKCEECDSIFKWFSDLTKHKRIH |
| O95780_HUMAN | 542 | YKCEKCDKVFKRFSYLTKHKRIH |
| O95779_HUMAN | 543 | YKCEKCDKVFKRFSYLTKHKRIH |
| O95780_HUMAN | 544 | CICEECGKTFKWFSYLTKHKRIH |
| O95779_HUMAN | 545 | CICEECGKTFKWFSYLTKEKRIH |
| ZN43_HUMAN | 546 | YKCEECGKAFNHFSILTKHKRIH |
| ZN91_HUMAN | 547 | YKCEKCCKAFNQSSILTNHKKIH |
| Q02313_HUMAN | 548 | YKCEKCVRAFNQASKLTEHKLIH |
| ZN85_HUMAN | 549 | YKSKECEKAFNQSSKLTEHKKIH |
| ZN43_HUMAN | 550 | YKCKECAKAFNQSSNLTEHKKIH |
| ZN85_HUMAN | 551 | YKCEECGKAFNQSSKLTKHKKIH |
| ZN85_HUMAN | 552 | YKCEECGKAFNQSSNLIKHKKIH |
| O43345_HUMAN | 553 | YKCEECGKAFNRSAILIKHKRIH |
| O43345_HUMAN | 554 | YKCEECGKAFNQSAILIKHKRIH |
| O43345 HUMAN | 555 | YKCEECGKAFNQSAILTKHKIIH |
| ZN43_HUMAN | 556 | YKCEVCGKAFNQFSNLTTHKRIH |
| ZN43_HUMAN | 557 | YTCEECGKAFNQFSNLTTHKRIH |
| O75346_HUMAN | 558 | YRCEECGKAFNQSANLTTHKRIH |
| ZN85_HUMAN | 559 | YTCEECGKAFNQSSNLTKHKRIH |
| Z141_HUMAN | 560 | YKCKDCDKAFKRFSHLNKHKKIH |
| Z141_HUMAN | 561 | YKCKECDKAFKQFSLLSQHKKIH |
| Q02313_HUMAN | 562 | YKCEECGKAFKQFSNLTDHKKIH |
| ZN43_HUMAN | 563 | YKCEECGKAFTQSSNLTTHKKIH |
| ZN43_HUMAN | 564 | YKCEECGKAFTQSSNLTTHKKIH |
| ZN85_HUMAN | 565 | YKCEECGRAFKQSSNLTTHKIIH |
| Q02313_HUMAN | 566 | YKCEECGKAFNQLSNLTRHKVIH |
| ZN85_HUMAN | 567 | YECEKCGKAFNQSSNLTRHKKSH |
| O95780_HUMAN | 568 | YNCEECGKAFNRCSHLTRHKKIH |
| O95779_HUMAN | 569 | YNCEECGKAFNRCSHLTRHKKIH |
| O95780_HUMAN | 570 | YTCEDCGRAFNRHSHLTKHKTIH |
| O95779_HUMAN | 571 | YTCEDCGRAFNRHSHLTKHKTIH |
| Q02313_HUMAN | 572 | YECEECGKAFNRSSKLTEHKYIH |
| ZN91_HUMAN | 573 | YKCEECGKAFNRSSNLTIHKFIH |
| ZN91_HUMAN | 574 | YKCEECGKAFNRSSNLTIHKFIH |
| ZN43_HUMAN | 575 | YKCEKCGKAFNRPSNLIEHKKIH |
| Z141_HUMAN | 576 | YTCEECRKIFTSSSNFAKHKRIH |
| Z141_HUMAN | 577 | FTCEECGSIFTTSSHFAKHKIIH |
| Z141_HUMAN | 578 | YTCEECGKAFKWSLIFNEHKRIH |
| Z141_HUMAN | 579 | YTCEECGKAFRQSSKLNEHKKVH |
| O43345_HUMAN | 580 | YKCEECGKAYKWSSTLSYHKKIH |
| O43345_HUMAN | 581 | YKCEECGKAYKWSSTLSYHKKIH |
| O43345_HUMAN | 582 | YKCEECGKAYKWPSTLSYHKKIH |
| O43345_HUMAN | 583 | YKCEECGKAYKWPSTLSYHKKIH |
| O43345_HUMAN | 584 | YKCEECGKAYKWPSTLRYHKKIH |
| O43345_HUMAN | 585 | YKCEECGKGFSWSSTLSYHKKIH |
| O43345_HUMAN | 586 | YKCEECGKAFSWLSVFSKHKKIH |
| O43345_HUMAN | 587 | YKCEECGKAFSWLSVFSKHKKTH |
| O95780_HUMAN | 588 | YKCEECGKAFHWCSPFVRHKKIH |
| O95779_HUMAN | 589 | YKCEECGKAFHWCSPFVRHKKIH |
| Z195_HUMAN | 590 | YTCEECGNIFKQLSDLTKHKKTH |
| Z195_HUMAN | 591 | YKCEECGRAFMWFSDITKHKQTH |
| O43345_HUMAN | 592 | YKCEECGKAFSWPSRLTEHKATH |
| O43345_HUMAN | 593 | YKCEECDKAFSWPSSLTEHKATH |
| ZN43_HUMAN | 594 | YKCEECGKAFKWSSKLTEHKITH |
| ZN43_HUMAN | 595 | YKCEECGKAFKWSSKLTEHKLTH |
| ZN91_HUMAN | 596 | YKCEECGKAFSHSSALAKHKRIH |
| ZN91 HUMAN | 597 | YKCEECGKAFSHSSALAKHKRIH |
| ZN91_HUMAN | 598 | YKCEECGKAFSHSSTLAKHKRIH |
| ZN91_HUMAN | 599 | YKCEECGKAFSQPSHLTTHKRMH |
| ZN91_HUMAN | 600 | YKCEECGKAFSQSSTLTRHKRLH |
| ZN91_HUMAN | 601 | YKCEECGKAFSQSSTLTRHTRMH |
| Z124_HUMAN | 602 | YECMECGKALGFSRSLNRHKRIH |
| Z141_HUMAN | 603 | YKCDECGKAFGRSRVLNEHKKIH |
| ZN74_HUMAN | 604 | YKCDECGKAFTWSTNLLEHRRIH |
| Z195_HUMAN | 605 | YKCDECGKAYTQSSHLSEHRRIH |
| Z195_HUMAN | 606 | YKCDECGKNFTQSSNLIVHKRIH |
| Z195_HUMAN | 607 | YKCDECGKNFTQSSNLIVHKRIH |
| ZN80_HUMAN | 608 | YKCKECGSVFNKNSLLVRHQQIH |
| Z165_HUMAN | 609 | FGCKECGRAFNLNSHLIRHQRIH |
| Q02313_HUMAN | 610 | YKCKECGKAFNQTSHLIRHKRIH |
| O60792_HUMAN | 611 | YKCNECGRAFNQNIHLTQHKRIH |
| ZN74_HUMAN | 612 | YRCGECGKAFNQRTHLTRHHRIH |
| Q15776_HUMAN | 613 | YKCKECGKAFNGNTGLIQHLRIH |
| O43309_HUMAN | 614 | YKCDECGNAFRGITSLIQHQRIH |
| O43309_HUMAN | 615 | YKCEECGKAFRGRTVLIRHKIIH |
| O75123_HUMAN | 616 | YVCNECGKRFSQTSNFTQHQRIH |
| O60792_HUMAN | 617 | YKCNECGKAFNGPSTFIRHHMIH |
| O43296_HUMAN | 618 | FVCSECGKAFTHCSTFILHKRAH |
| O43337_HUMAN | 619 | YECSQCRKAFTHRSTFIRHNRTH |
| O43296_HUMAN | 620 | YKCNECGKAFTHRSNFVLHNRRH |
| OZF_HUMAN | 621 | YGCNECGKAFSQFSTLALHLRIH |
| ZN83_HUMAN | 622 | YKCNERGKAFHQGLHLPIHQIIH |
| ZN07_HUMAN | 623 | YKCNECGKAFSQNSTLFQHQIIH |
| ZN83_HUMAN | 624 | YKCNECGKVFSRNSYLAQHLIIH |
| ZN83_HUMAN | 625 | YECNKCGKVFSRNSYLVQHLIIH |
| ZN83_HUMAN | 626 | YKCNECGKVFGLNSSLAHHRKIH |
| ZN83_HUMAN | 627 | YKCNECGKVFHQISHLAQHRTIH |
| ZN83_HUMAN | 628 | YKCNECGKVFHNMSHLAQHRRIH |
| ZN83_HUMAN | 629 | YKCNECGKVFNQISHLAQHQRIH |
| ZN83_HUMAN | 630 | YRCNVCGKVFHHISHLAQHQRIH |
| ZN83_HUMAN | 631 | YKCDECGKVFSQNSYLAYHWRIH |
| Z189_HUMAN | 632 | YKCDECGKTFSVSAHLVQHQRIH |
| O75802_HUMAN | 633 | YKCDECGKTFSVSAHLVQHQRIH |
| ZN83_HUMAN | 634 | YKCDECDRAFSQNSHLVQHHRIH |
| O60792_HUMAN | 635 | YKCDECGKAFSQRTHLVQHQRIH |
| O43361_HUMAN | 636 | YECGESSKVFKYNSSLIKHQIIH |
| ZN83_HUMAN | 637 | FKCNECGKAFSMRSSLTNHHAIH |
| O60792_HUMAN | 638 | YKCNECGKAFSYCSSLTQHRRIH |
| Z137_HUMAN | 639 | YKYHDCGKVFSQASSYAKHRRIH |
| O14709_HUMAN | 640 | YKCEDCGKAFSYNSSLLVHRRIH |
| Z124_HUMAN | 641 | YVCMECGKAFSCLSSLQGHIKAH |
| O60792_HUMAN | 642 | YQCHECGKTFSYGSSLIQHRKIH |
| O60792_HUMAN | 643 | YDCAECGKSFSYWSSLAQHLKIH |
| ZN83_HUMAN | 644 | YKCNECGKVFSHKSSLVNHWRIH |
| ZN83_HUMAN | 645 | YKCNECGKVFSHKSSLVNHWRIH |
| Z132_HUMAN | 646 | YKCSECGKFFSRKSSLICHWRVH |
| O43339_HUMAN | 647 | YKCNECGKFFSQTSHLNDHRRIH |
| O43338_HUMAN | 648 | YECSECGKSFSQTSHLNDHRRIH |
| ZN45_HUMAN | 649 | YKCNACGKSFSYSSHLNIHCRIH |
| ZN45_HUMAN | 650 | YKCGTCGKGFSRSSDLNVHCRIH |
| Z263_HUMAN | 651 | YKCPLCGKNFSNNSNLIRHQRIH |
| Z202_HUMAN | 652 | YTCPTCGKSFSRGYHLIRHQRTH |
| O75850_HUMAN | 653 | FSCPQCGKSFSRKTHLVRHQLIH |
| Z205_HUMAN | 654 | YACPLCGKSFSRRSNLHRHEKIH |
| O15535_HUMAN | 655 | HQCIECGKSFNRHCNLIRHQKIH |
| ZN24_HUMAN | 656 | YECVQCGKSYSQSSNLFRHQRRH |
| Z191_HUMAN | 657 | YECVQCGKSYSQSSNLFRHQRRH |
| Q99592_HUMAN | 658 | YTCTQCGKSFQYSHNLSRHAVVH |
| Q13397_HUMAN | 659 | YTCTQCGKSFQYSHNLSRHAVVH |
| Z189_HUMAN | 660 | YLCRQCGKSFSQLCNLIRHQGVH |
| O75802_HUMAN | 661 | YLCRQCGKSFSQLCNLIRHQGVH |
| Z189_HUMAN | 662 | YQCKECGKSFSQLCNLTRHQRIH |
| O75802_HUMAN | 663 | YQCKECGKSFSQLCNLTRHQRIH |
| Z263_HUMAN | 664 | YKCTLCGENFSHRSNLIRHQRIH |
| Z263_HUMAN | 665 | YKCPECGEIFAHSSNLLRHQRIH |
| O95878_HUMAN | 666 | YKCSECGKSFSRSSNRIRHERIH |
| Z263_HUMAN | 667 | YTCHECGDSFSHSSNRIRHLRTH |
| O43336_HUMAN | 668 | YVCIICGKSFIRSSDYMRHQRIH |
| O43336_HUMAN | 669 | YVCMECGKSFIHSYDRIRHQRVH |
| BCL6_HUMAN | 670 | YRCNICGAQFNRPANLKTHTRIH |
| Z133_HUMAN | 671 | YKCGECGLSFSKMTNLLSHQRIH |
| ZN75_HUMAN | 672 | YRCSWCGKSFSHNTNLHTHQRIH |
| O60893_HUMAN | 673 | YKCNECERSFTRNRSLIEHQKIH |
| ZN74_HUMAN | 674 | YKCSECGRAFSQNHCLIKHQKIH |
| O14709_HUMAN | 675 | YACSECGKGFTYNRNLIEHQRIH |
| Z177_HUMAN | 676 | YKCFQCEKAFSTSTNLIMHKRIH |
| O60792_HUMAN | 677 | YKCNECEKAFSRSENLINHQRIH |
| O94892_HUMAN | 678 | YGCTLCAKVFSRKSRLNEHQRIH |
| Z189_HUMAN | 679 | YHCTKCKKSFSRNSLLVEHQRIH |
| O75802_HUMAN | 680 | YHCTKCKKSFSRNSLLVEHQRIH |
| O43309_HUMAN | 681 | YQCTQCNKSFSRRSTLTQHQGVH |
| O15535_HUMAN | 682 | YQCSQCSKSYSRRSFLIEHQRSH |
| Z205_HUMAN | 683 | YTCPACRKSFSHHSTLIQHQRIH |
| Z189_HUMAN | 684 | YTCIECGKSFSRSSFLIEHQRIH |
| O75802_HUMAN | 685 | YTCIECGKSFSRSSFLIEHQRIH |
| Z189_HUMAN | 686 | FQCNECGKSFSRSSFVIEHQRIH |
| O75802_HUMAN | 687 | FQCNECGKSFSRSSFVIEHQRIH |
| Z189_HUMAN | 688 | YLCTVCGKSFSRSSFLIEHQRIH |
| O75802_HUMAN | 689 | YLCTVCGKSFSRSSFLIEHQRIH |
| O14709_HUMAN | 690 | YECHVCRKVLTSSRNLMVHQRIH |
| O14709_HUMAN | 691 | YECDKCRKSFTSKRNLVGHQRIH |
| ZN35_HUMAN | 692 | YECNECGKTFTRSSNLIVHQRIH |
| O75123_HUMAN | 693 | YECNECGKSFIRSSSLIRHYQIH |
| O43296_HUMAN | 694 | YECVECGKSFCWSTNLIRHAIIH |
| O43296_HUMAN | 695 | YECSECGKVFLESAALIHHYVIH |
| O43337_HUMAN | 696 | YECTQCGKAFHRSTYLIQHSVIH |
| O43296_HUMAN | 697 | YECTECGKTFIKSTHLLQHHMIH |
| O75290_HUMAN | 698 | YECKECGKYFSRSANLIQHQSIH |
| Z205_HUMAN | 699 | YACTDCGKRFGRSSHLIQHQIIH |
| Z165_HUMAN | 700 | YECSECGKTFRVSSHLIRHFRIH |
| Q15776_HUMAN | 701 | YECDECGKTFRRSSHLIGHQRSH |
| Q15776_HUMAN | 702 | YECNECGKAFSHSSHLIGHQRIH |
| Z189_HUMAN | 703 | YECNYCCKTFSVSSTLIRHQRIH |
| O75802_HUMAN | 704 | YECNYCGKTFSVSSTLIRHQRIH |
| O43337_HUMAN | 705 | YECNACGKAFSQSSTLIRHYLIH |
| ZN07_HUMAN | 706 | YECSECGKAFSRSSYLIEHQRIH |
| Z132_HUMAN | 707 | YECSECGKAFAHSSTLIEHWRVH |
| O43340_HUMAN | 708 | YECSECGKAFSCNIYLIHHQRFH |
| Z135_HUMAN | 709 | YECGECGKAFSQSTLLTEHRRIH |
| O43338_HUMAN | 710 | YECGECGKSFSQSSNLIEHCRIH |
| O43338_HUMAN | 711 | YECGKCGKSFTQHSGLILHRKSH |
| Z140_HUMAN | 712 | YECDECGKVFTWHASLIQHTKSH |
| Q13398_HUMAN | 713 | YACPECGKSFSQIYSLNSHRKVH |
| Q13398_HUMAN | 714 | YECSKCGKSFKQSSSFSSHRKVH |
| O43340_HUMAN | 715 | YECSECGKSFSHSTNLFRHWRVH |
| O43340_HUMAN | 716 | YECSECGKSFSHSTNLYRHRSAH |
| O43340_HUMAN | 717 | YECSECGKSFSQSSGLLRHRRVH |
| O43340_HUMAN | 718 | YKCSECGKSFSQSSGFLRHRKAH |
| O43340_HUMAN | 719 | YECSECQKVFSQSSGLFRHRRAH |
| O43340_HUMAN | 720 | YECDECGKSYSQSSALLQHRRVH |
| Q13398_HUMAN | 721 | YECSECGKSFSQSSSLIQHRRVH |
| Q13398_HUMAN | 722 | YECGECGKSFSQRSNLMQHRRVH |
| Z132_HUMAN | 723 | YECSECRKSFSRSSSLIQHWRIH |
| Z132_HUMAN | 724 | YECSQCGKSFSRSSALIQHWRVH |
| Q13398_HUMAN | 725 | HECNECGKSFSRSSSLIHHRRLH |
| O43339_HUMAN | 726 | YKCGECGNSFSQSAILNQHRRIH |
| O43339_HUMAN | 727 | YKCGDCGKSFSQSSILIQHRRIH |
| O60765_HUMAN | 728 | YRCEECGISFGQSSALIQHRRIH |
| O43338_HUMAN | 729 | YECGQCGKSFSLKCGLIQHQLIH |
| O43339_HUMAN | 730 | YECGQCGKSFSQKSGLIQHQVVH |
| O43338_HUMAN | 731 | YDCGQCGKSFIQKSSLIQHQVVH |
| Q13398_HUMAN | 732 | YQCSQCGKSFGCKSVLIQHQRVH |
| O43340_HUMAN | 733 | YVCSECGKSFGQKSVLIQHQRVH |
| O43340_HUMAN | 734 | YDCSECGKSFRQVSVLIQHQRVH |
| Q13398_HUMAN | 735 | YECSECSKSFSCKSNLIKHLRVH |
| O43339_HUMAN | 736 | YECGQCGKSFSQKATLIKHQRVH |
| O43338_HUMAN | 737 | YVCGQCGKSFSQRATLIKHHRVH |
| O43339_HUMAN | 738 | YECSQCGKSFSQKATLVKHQRVH |
| Q13398_HUMAN | 739 | YECSECGKSFSQNFSLIYHQRVH |
| O43340_HUMAN | 740 | YECSVCGKSFIRKTHLIRHQTVH |
| O43340 HUMAN | 741 | YECSECEKSFSCKTDLIRHQTVH |
| O43340_HUMAN | 742 | YECRECGKSFTRKNHLIQHKTVH |
| Z189_HUMAN | 743 | HKCEECGKGFVRKAHFIQHQRVH |
| O75802_HUMAN | 744 | HKCEECGKGFVRKAHFIQHQRVH |
| O43340_HUMAN | 745 | HECSECGKSFSRKTHLTQHQRVH |
| O43309_HUMAN | 746 | YQCKECGKSFSQSGLIQHQRIH |
| Q15776_HUMAN | 747 | YQCNQCGKAFSQSAGLILHQRIH |
| O15535_HUMAN | 748 | YHCKECGKVFSQSAGLIQHQRIH |
| O60792_HUMAN | 749 | YNCNECRKTFSQSTYLIQHQRIH |
| Q15776_HUMAN | 750 | YHCKECGKAFSQNTGLILHQRIH |
| ZN84_HUMAN | 751 | YGCNECGRAFSEKSNLINHQRIH |
| Q15776_HUMAN | 752 | YKCNECGRAFSQKSGLIEHQRIH |
| Z189_HUMAN | 753 | HKCDECGKAFSRNSGLIQHQRIH |
| O75802_HUMAN | 754 | HKCDECGKAFSRNSGLIQHQRIH |
| Z189_HUMAN | 755 | HKCEECGKAFSRSSGLIQHQRIH |
| O75802_HUMAN | 756 | HKCEECGKAFSRSSGLIQHQRIH |
| ZN24_HUMAN | 757 | YKCLECGKAFSQNSGLINHQRIH |
| Z191_HUMAN | 758 | YKCLECGKAFSQNSGLINHQRIH |
| OZF_HUMAN | 759 | YQCSECGKAFSQKSHHIRHQKIH |
| Q15776_HUMAN | 760 | YQCNECGKAFIQRSSLIRHQRIH |
| ZN35_HUMAN | 761 | YDCSECGKAFSQLSSLIVHQRIH |
| ZN07_HUMAN | 762 | YRCEECGKAFGQSSSLIHHQRIH |
| O60765_HUMAN | 763 | FKCNTCGKTFRQSSSRIAHQRIH |
| OZF_HUMAN | 764 | FKCSECGTAFGQKKYLIKHQNIH |
| OZF_HUMAN | 765 | FECNECGKAFSQKQYVTKHQNTH |
| Q92951_HUMAN | 766 | FECTHCGKSFRAKGNLVTHQRIH |
| OZF_HUMAN | 767 | FECNECGKSFSQKENLLTHQKIH |
| ZN74_HUMAN | 768 | FKCNECGKAFSSHAYLIVHRRIH |
| ZN74_HUMAN | 769 | FKCADCGKGFSCHAYLLVHRRIH |
| O60765_HUMAN | 770 | FKCSECGRAFSQSASLIQHERIH |
| ZN35_HUMAN | 771 | FECHECGKAFIQSANLVVHQRIH |
| ZN35_HUMAN | 772 | FTCSVCGKGFSQSANLVVHQRIH |
| ZN35_HUMAN | 773 | FACNDCGRAFTQSANLIVHQRSH |
| O14709_HUMAN | 774 | YKCNECGKDFSQNKNLVVHQRMH |
| O14709_HUMAN | 775 | YKCDECGKTFAQTTYLIDHQRLH |
| O14709_HUMAN | 776 | YKCNECGRVFSQNAYLIDHQRLH |
| O14709_HUMAN | 777 | YKCTECGKAFTQSAYLFDHQRLH |
| O14709_HUMAN | 778 | YKCNECGKAFSQSAYLLNHQRIH |
| Z157_HUMAN | 779 | YQCNECGKSFRVHSSLGIHQRIH |
| O60765_HUMAN | 780 | YNCNECGKALSSHSTLIIHERIH |
| EVI1_HUMAN | 781 | YKCDQCPKAFNWKSNLIRHQMSH |
| Q15776_HUMAN | 782 | YQCNVCGKAFSYRSALLSHQDIH |
| O43309_HUMAN | 783 | YECNECGKAFVYNSSLVSHQEIH |
| Z200_HUMAN | 784 | YGCKKCGRRFGRLSNCTRHEKTH |
| O15361_HUMAN | 785 | YGCKKCGRRFGRLSNCTRHEKTH |
| ZN07_HUMAN | 786 | YKCNDCGKAFNRSSRLTQHQKIH |
| ZN74_HUMAN | 787 | YQCGSCGKAFTCHSSLTVHEKIH |
| ZN35_HUMAN | 788 | YVCSKCGKAFTQSSNLTVHQKIH |
| Z140_HUMAN | 789 | YECIECGKAFRRFSHLTRHQSIH |
| O60893_HUMAN | 790 | YQCNMCGKAFRRNSHLLRHQRIH |
| Q13396_HUMAN | 791 | YSCTECEKSFVQKQHLLQHQKIH |
| O43361_HUMAN | 792 | YECTQCAKAFVRKSHLVQHEKIH |
| O43361_HUMAN | 793 | YECTECEKAFVRKSHLVQHQKIH |
| O75123_HUMAN | 794 | YECKECGKAFLQKAHLTEHQKIH |
| O75290_HUMAN | 795 | YECKECGKGFNRGAHLIQHQKIH |
| O75290_HUMAN | 796 | YECKECGKGFNRGAHLIQHQKIH |
| O75290_HUMAN | 797 | FECKECGKAFRLHMQLIRHQKLH |
| O75290_HUMAN | 798 | FECKECGKAFRLHMHLIRHQKLH |
| O75290_HUMAN | 799 | FECKECGKAFRLHIQFTRHQKFH |
| O75290_HUMAN | 800 | YECKECGKAFRLYLQLSQHQKTH |
| Z140_HUMAN | 801 | YECTECGKAFSRASNLTRHQRIH |
| O43296_HUMAN | 802 | YECVECGKAFTRNSGLTRHKRIH |
| O43296_HUMAN | 803 | YECMECGKAFNRKSYLTQHQRIH |
| O14913_HUMAN | 804 | HECVECGKRFSSSSRLQEHQKIH |
| EVI1_HUMAN | 805 | HACPECGKTFATSSGLKQHKHIH |
| O15535_HUMAN | 806 | YECNECGKAFSRSSGLFNHRGIH |
| Z132_HUMAN | 807 | YECNDCGKAFSNSSTLIQHQKVH |
| Z132_HUMAN | 808 | YECIQCGKAFSERSTLVRHQKVH |
| Z132_HUMAN | 809 | YECDECGKAFSNRSHLIRHEKVH |
| Z124_HUMAN | 810 | YECQKCGKAFSRASTLWKHKKTH |
| ZN35_HUMAN | 811 | FKCNECEKAFSYSSQLARHQKVH |
| O60792_HUMAN | 812 | FECSECGKAFSYLSNLNQHQKTH |
| O75467_HUMAN | 813 | FRCSECGKAFSHGSNLSQHRKIH |
| O75467_HUMAN | 814 | FACPQCGRAFSHSSNLTQHQLLH |
| OZF_HUMAN | 815 | FACKVCGKVFSHKSNLTEHEHFH |
| Z132_HUMAN | 816 | YECSQCGKLFSHLCNLAQHKKIH |
| O60765_HUMAN | 817 | YECNTCGKLFNHRSSLTNHYKIH |
| O60792_HUMAN | 818 | YECAECGKAFRHCSSLAQHQKTH |
| O43336_HUMAN | 819 | CECSECGKCFRHRTSLIQHQKVH |
| O43336_HUMAN | 820 | CECNECGKVFSHQKRLLEHQKVH |
| O95878_HUMAN | 821 | YECTECGRTFSDISNFGAHQRTH |
| O60792_HUMAN | 822 | YECNECGKAFSQHSNLTQHQKTH |
| O43309_HUMAN | 823 | YHCNDCGKAFSQKAGLFHHIKIH |
| O43336_HUMAN | 824 | YECSDCGKAFISKQTLLKHHKIH |
| O60893_HUMAN | 825 | YECDDCGKTFSQSCSLLEHHKIH |
| O43338_HUMAN | 826 | FECDECGKSFSQRTTLNKHHKVH |
| O75123_HUMAN | 827 | YVCSYCGKGFIQRSNFLQHQKIH |
| O60792_HUMAN | 828 | YTCNECGKAFSQRGHFMEHQKIH |
| ZN42_HUMAN | 829 | YTCDVCGKVFSQRSNLLRHQKIH |
| O14709_HUMAN | 830 | YGCNDCSKVFRQRKNLTVHQKIH |
| O43361_HUMAN | 831 | YVCSECGKAFLTQAHLDGHQKIQ |
| O43361_HUMAN | 832 | YTCSECGKAFLTQAHLVGHQKIH |
| O43361_HUMAN | 833 | YECTQCGKAFLTQAHLVGHQKTH |
| Z157_HUMAN | 834 | YECGECAKTFSARSYLIAHQKTH |
| O75123_HUMAN | 835 | YECNECGKAFFLSSYLIRHQKIH |
| Q13398_HUMAN | 836 | YECNECGKFFTYYSSFIIHQRVH |
| O43361_HUMAN | 837 | YKCSKCGKFFRYRCTLSRHQKVH |
| O43361_HUMAN | 838 | YECNKCGKFFMYNSKLIRHQKVH |
| Z132_HUMAN | 839 | YECNECGKFFSQNSILIKWQKVH |
| Q13396_HUMAN | 840 | YECGYCGKSFSHPSDLVRHQRIH |
| O75467_HUMAN | 841 | YACPVCGKAFRHSSSLVRHQRIH |
| Z165_HUMAN | 842 | HQCNECGKAFRHSSKLARHQRIH |
| Z205_HUMAN | 843 | YHCLDCGKSFSHSSHLTAHQRTH |
| Z135_HUMAN | 844 | YACRDCGKAFTHSSSLTKHQRTH |
| Z135_HUMAN | 845 | YECNDCGKAFSHSSSLTKHQRIH |
| Z135_HUMAN | 846 | YQCGECGKAFSHSSSLTKHQRIH |
| ZN74_HUMAN | 847 | FDCSQCWKAFSCHSSLIMHQRIH |
| ZN74_HUMAN | 848 | YTCGECGKAFSCHSSLNVHQRIH |
| ZN35_HUMAN | 849 | YECKECGKAFSCFSHLIVHQRIH |
| O43309_HUMAN | 850 | YKCNECGKAFGRWSALNQHQRLH |
| ZN24_HUMAN | 851 | YGCVECGKAFSRSSILVQHQRVH |
| Z191_HUMAN | 852 | YGCVECGKAFSRSSILVQHQRVH |
| O43296_HUMAN | 853 | YKCSECGKAFSRSSSLTQHQRMH |
| ZN75_HUMAN | 854 | FKCQECGKSFRVSSDLIKHHRIH |
| O75290_HUMAN | 855 | FVCKBCGMAFRYHYQLIEHCQIH |
| O75467_HUMAN | 856 | FVCTQCGRAFRERPALFHHQRIH |
| ZN74_HUMAN | 857 | FKCEKCGEMFNWSSHLTEHQRLH |
| ZN85_HUMAN | 858 | FKCTKCGKSFGMISCLTEHSRIH |
| ZN43_HUMAN | 859 | FKCKECGKSFCMLPHLAQHKIIH |
| Z195_HUMAN | 860 | FKCQECGKSFQMLSFLTEHQKIH |
| ZN07_HUMAN | 861 | FKCDECGKAFRWISRLSQHQLIH |
| Z189_HUMAN | 862 | HKCGECGKAFRLSTYLIQHQKIH |
| O75802_HUMAN | 863 | HKCGECGKAFRLSTYLIQHQKIH |
| ZN07_HUMAN | 864 | FKCTECGKAFRLSSKLIQHQRIH |
| O75290_HUMAN | 865 | FECKECGKAFTLLTKLVRHQKIH |
| O75290_HUMAN | 866 | FECKECGKVFSLPTQLNRHKNIH |
| O75290_HUMAN | 867 | FECRECGKAFSLLNQLNRHKNIH |
| O75290_HUMAN | 868 | FECKECEKAFSNRAHLIQHYIIH |
| O43296_HUMAN | 869 | FECKECGKAFSNRKDLIRHFSIH |
| O62425_CAEEL | 870 | FVCKVCGKAFRQASTLCRHKIIH |
| O75123_HUMAN | 871 | FECKDCGKAFIQSSKLLLHQIIH |
| O75290_HUMAN | 872 | FECKECGKFFRRGSNLNQHRSIH |
| O75290_HUMAN | 873 | FECKECGKSFNRSSNLVQHQSIH |
| O75290_HUMAN | 874 | FECKECGKSFNRSSNLVQHQSIH |
| O75290_HUMAN | 875 | FECQDCGKAFNRGSSLVQHQSIH |
| O94892_HUMAN | 876 | FVCSECRKAFSSKRNLIVHQRTH |
| O14709_HUMAN | 877 | FECSECGRAFSSNRNLIEHKRIH |
| Z135_HUMAN | 878 | YECNQCGRASARATLLIEHQRIH |
| Z157_HUMAN | 879 | FECQECGKAFCRKAHLTEHQRTH |
| Z157_HUMAN | 880 | FECNECGKAYCRKSNLVEHLRIH |
| O75123_HUMAN | 881 | FECNECGKAFIRSSKLIQHQRIH |
| ZN42_HUMAN | 882 | FRCAECGQSFRQRSNLLQHQRIH |
| ZN42_HUMAN | 883 | FACPECGQSFRQHANLTQHRRIH |
| ZN42_HUMAN | 884 | FACAECGQSFRQRSNLTQHRRIH |
| ZN42_HUMAN | 885 | --CAECGKAFRQRPTLTQHLRVH |
| ZN42_HUMAN | 886 | YACPECGKAFRQRPTLTQHLRTH |
| O14913_HUMAN | 887 | YKCEECGNSFYYPAMLKQHQRIH |
| Z174_HUMAN | 888 | YTCGECGNCFGRQSTLKLHQRIH |
| PLZF_HUMAN | 889 | YECEFCGSCFRDESTLKSHKRIH |
| BCL6_HUMAN | 890 | YPCEICGTRFRHLQTLKSHLRIH |
| O43296_HUMAN | 891 | FECLECGKAFNHRSYLKRHQRIH |
| O43337_HUMAN | 892 | YKCLECGKAFKRRSYLMQHHPIH |
| O43296_HUMAN | 893 | YECLECGKVFKHRSYLMWHQQTH |
| O75123_HUMAN | 894 | YECKECGKAFRHRSDLIEHQRIH |
| O43336_HUMAN | 895 | YECKECGKAFIHKKRLLEHQRIH |
| Z157_HUMAN | 896 | YECSECGNAFYVKVRLIEHQRIH |
| Z157_HUMAN | 897 | YECNECGNAFYVKARLIEHQRMH |
| OZF_HUMAN | 898 | FVCKECGKTFSGKSNLTEHEKIH |
| Z134_HUMAN | 899 | YKCSDCGKVFRHKSTLVQHESIH |
| O60893_HUMAN | 900 | YECEDCGKTFIGSSALVIHQRVH |
| O43339_HUMAN | 901 | YECSECGKLFRQNSSLVDHQKIH |
| O43338_HUMAN | 902 | FECSECGKFFRQSYTLVEHQKIH |
| O43338_HUMAN | 903 | YECGECGKLFRQSFSLVVHQRIH |
| O43361_HUMAN | 904 | YECSECGKLFMDSFTLGRHQRVH |
| O43361_HUMAN | 905 | YECSECGKFFRDSYKLIIHQRVH |
| O43361_HUMAN | 906 | YECNECGKFFLDSYKLVIHQRIH |
| O43336_HUMAN | 907 | YECSECGKGFYLEVKLLQHQRIH |
| ZN07_HUMAN | 908 | YECAECGKVFRLCSQLNQHQRIH |
| Z132_HUMAN | 909 | HVCKECGKAFSHSSKLRKHQKFH |
| TYY1_HUMAN | 910 | HVCAECGKAFVESSKLKRHQLVH |
| O15391_HUMAN | 911 | HVCAECGKAFLESSKLRRHQLVH |
| O94892_HUMAN | 912 | HVCSECGKAFVKKSQLTDHERVH |
| ZFX_HUMAN | 913 | HICVECGKGFRHPSELKKHMRIH |
| ZFY_HUMAN | 914 | HICVECGKGFRYPSELRKHMRIH |
| Q15558_HUMAN | 915 | HICVECGKGFRHPSELRKHMRIH |
| Z135_HUMAN | 916 | YECHECLKGFRNSSALTKHQRIH |
| ZN74_HUMAN | 917 | YTCGECGKAFRQSSSLTLHRRWH |
| Z174_HUMAN | 918 | YQCGQCGKSFRQSSNLHQHHRLH |
| Z195_HUMAN | 919 | YQCEECGKVFRTCSSLSNHKRTH |
| HKR3_HUMAN | 920 | FQCHLCGKTFRTQASLDKHNRTH |
| O43337_HUMAN | 921 | YDCMACGKAFRCSSELIQHQRIH |
| O60765_HUMAN | 922 | YLCNECGNTFKSSSSLRYHQRIH |
| O60765_HUMAN | 923 | YKCNECGKTFRCNSSLSNHQRIH |
| Z140_HUMAN | 924 | YKCNECGKAFSSGSELIRHQITH |
| Q14585_HUMAN | 925 | YECKECGKAFSFGSGLIRHQIIH |
| Q14585_HUMAN | 926 | YICNECGKAFSFGSALTRHQRIH |
| Q14585_HUMAN | 927 | YECKECGKSFSSGSALNRHQRIH |
| Q14585_HUMAN | 928 | YECKACGMAFSSGSALTRHQRIH |
| Q14585_HUMAN | 929 | YECKECGKSFSFESALIRHHRIH |
| Q14585_HUMAN | 930 | YECKECGKTFSSGSDLTQHHRIH |
| Q14585_HUMAN | 931 | YVCKECGKAFNSGSDLTQHQRIH |
| Q14585_HUMAN | 932 | YECKECGKAFYSGSSLTQHQRIH |
| Q14585 HUMAN | 933 | FECKECGKAFGSGSNLTHHQRIH |
| Q14585_HUMAN | 934 | YECKECGKAFGSGANLAYHQRIH |
| Q14585_HUMAN | 935 | YECIDCGKAFGSGSNLTQHRRIH |
| Q14585_HUMAN | 936 | YECKECGKAFGSGSKLIQHQLIH |
| Q14585_HUMAN | 937 | YECKECEKAFRSGSKLIQHQRMH |
| ZN80_HUMAN | 938 | YECKECGKTFYYNSSLTRHMKIH |
| ZN80_HUMAN | 939 | YECKECGKGFYYSYSLTRHTRSH |
| Z165_HUMAN | 940 | YECNECGKSFAESSDLTRHRRIH |
| Z202_HUMAN | 941 | YKCTICGKSFSQKSVLTTHQRIH |
| O43167_HUMAN | 942 | YTCEICGKSFTAKSSLQTHIRIH |
| Q92618_HUMAN | 943 | HTCCICGKSFPFQSSLSQHMRKH |
| Q15776_HUMAN | 944 | HKCDECGKSFAQSSGLVRHWRIH |
| O15535_HUMAN | 945 | HKCDECGKSFTQSSGLIRHQRIH |
| O60893_HUMAN | 946 | HYCHECGKSFAQSSGLTKHRRIH |
| ZN24_HUMAN | 947 | HICDECGKHFSQGSALILHQRIH |
| Z191_HUMAN | 948 | HICDECGKHFSQGSALILHQRIH |
| Z140_HUMAN | 949 | YACKECGKTFSQISNLVKHQMIH |
| Q14585_HUMAN | 950 | YECKECGKDFSFVSVINRHQRIH |
| O75123_HUMAN | 951 | FECKECGKGFSQSSLLIRHQRIH |
| UKLF_HUMAN | 952 | FKCNHCDRCFSRSDHLALHMKRH |
| O95600_HUMAN | 953 | FRCTDCNRSFSRSDHLSLHRRRH |
| SP2_HUMAN | 954 | YACAQCQKRFMRSDHLTKHYKTH |
| SP4_HUMAN | 955 | YACPECSKRFMRSDHLSKHVKTH |
| O60402_HUMAN | 956 | YACPECSKRFMRSDHLSKHVKTH |
| O75411_HUMAN | 957 | YACPMCDRRFMRSDHLTKHARRH |
| Q13118_HUMAN | 958 | YACPMCDRRFMRSDHLTKHARRH |
| O14901_HUMAN | 959 | YACPVCDRRFMRSDHLTKHARRH |
| BTE1_HUMAN | 960 | YACPLCEKRFMRSDHLTKHARRH |
| SP2_HUMAN | 961 | FVCNWFFCGKRFTRSDELQRHARTH |
| SP4_HUMAN | 962 | FICNWMFCGKRFTRSDELQRHRRTH |
| O60402_HUMAN | 963 | FICNWMFCGKRFTRSDELQRHRRTH |
| EZF_HUMAN | 964 | YHCDWDGCGWKFARSDELTRHYRKH |
| O95600_HUMAN | 965 | YKCTWDGCSWKFARSDELTRHFRKH |
| UKLF_HUMAN | 966 | YKCSWEGCEWRFARSDELTRHYRKH |
| EKLF_HUMAN | 967 | YACTWEGCGWRFARSDELTRHYRKH |
| BTE2_HUMAN | 968 | YKCTWEGCDWRFARSDELTRHYRKH |
| O14901_HUMAN | 969 | FNCSWDGCDKKFARSDELSRHRRTH |
| Q13118_HUMAN | 970 | FSCSWKGCERRFARSDELSRHRRTH |
| O75411_HUMAN | 971 | FSCSWKGCERRFARSDELSRHRRTH |
| BTE1_HUMAN | 972 | FPCTWPDCLKKFSRSDELTRHYRTH |
| EGR4_HUMAN | 973 | FACPVESCVRSFARSDELNRHLRIH |
| EGR2_HUMAN | 974 | YPCPAEGCDRRFSRSDELTRHIRIH |
| EGR1_HUMAN | 975 | YACPVESCDRRFSRSDELTRHIRIH |
| EGR3_HUMAN | 976 | HACPAEGCDRRFSRSDELTRHLRIH |
| Q16256_HUMAN | 977 | YQCDFKDCERRFFRSDQLKRHQRRH |
| WT1_HUMAN | 978 | YQCDFKDCERRFSRSDQLKRHQRRH |
| Q15881_HUMAN | 979 | YQCDFKDCERRFSRSDQLKRHQRRH |
| Q15881_HUMAN | 980 | FQCKACQRKFSRSDHLKTHTRTH |
| Q16256_HUMAN | 981 | FQCKTCQRKFSRSDHLKTHTRTH |
| WT1_HUMAN | 982 | FQCKTCQRKFSRSDHLKTHTRTH |
| EGR4_HUMAN | 983 | FQCRICLRNFSRSDHLTSHVRTH |
| EGR3_HUMAN | 984 | FQCRICMRSFSRSDHLTTHIRTH |
| EGR2_HUMAN | 985 | FQCRICMRNFSRSDHLTTHIRTH |
| EGR1_HUMAN | 986 | FQCRICMRNFSRSDHLTTHIRTH |
| EVI1_HUMAN | 987 | YTCRYCGKIFPRSANLTRHLRTH |
| O95878_HUMAN | 988 | YRCTVCGKHFSRSSNLIRHQKTH |
| Z140_HUMAN | 989 | YVCKVCNKSFSWSSNLAKHQRTH |
| O60893_HUMAN | 990 | YECEECGKVFSHSSNLIKHQRTH |
| Z135_HUMAN | 991 | YECSECGKSFSFRSSFSQHERTH |
| O95878_HUMAN | 992 | YICCECGKSPSNSSSFGVHHRTH |
| ZN80_HUMAN | 993 | CKCSECGKTFTYRSVFFRHSMTH |
| ZN80_HUMAN | 994 | YECSECGKTFSYHSVFIQHRVTH |
| Z135_HUMAN | 995 | YGCNECGKSFSHSSSLSQHERTH |
| Z135_HUMAN | 996 | YGCNECGKTFSHSSSLSQHERTH |
| Z263_HUMAN | 997 | YKCPECGKSFSRSSHLVIHERTH |
| Z263_HUMAN | 998 | YKCSECGESFSRSSRLMSHQRTH |
| Z202_HUMAN | 999 | CRCNECGKSFSRRDHLVRHQRTH |
| ZN74_HUMAN | 1000 | FKCSDCEKAFNSRSRLTLHQRTH |
| ZN42_HUMAN | 1001 | FACPECGQRFSQRLKLTRHQRTH |
| Z205_HUMAN | 1002 | YPCPECGKCFSQRSNLIAHNRTH |
| ZN75_HUMAN | 1003 | FKCDECGKRFIQNSHLIKHQRTH |
| ZN07_HUMAN | 1004 | FKCDECGKGFVQGSHLIQHQRIH |
| O15090_HUMAN | 1005 | YPCPLCGKRFRFNSILSLHMRTH |
| O94892_HUMAN | 1006 | YRCSECGKGFIVNSGLMLHQRTH |
| O95270_HUMAN | 1007 | HKCQVCGKAFSQSSNLITHSRKH |
| GFI1_HUMAN | 1008 | HKCQVCGKAFSQSSNLITHSRKH |
| Z135_HUMAN | 1009 | YKCQECGKAFSHSSALIEHHRTH |
| O60765_HUMAN | 1010 | FKCKECSKAFSQSSALIQHQITH |
| O60765_HUMAN | 1011 | CKCKVCGKAFRQSSALIQHQRMH |
| O60792_HUMAN | 1012 | CKCNECGKAFSYCSALIRHQRTH |
| Z151_HUMAN | 1013 | YVCERCGKRFVQSSQLANHIRHH |
| EVI1_HUMAN | 1014 | YECENCAKVFTDPSNLQRHIRSQH |
| Z205_HUMAN | 1015 | YVCDRCAKRFTRRSDLVTHQGTH |
| Z205_HUMAN | 1016 | HKCPICAKCFTQSSALVTHQRTH |
| Z124_HUMAN | 1017 | YGCTICEKVFNIPSSFQIHQRNH |
| Z200_HUMAN | 1018 | YTCPLCGKQFNESSYLISHQRTH |
| O15361_HUMAN | 1019 | YTCPLCGKQFNESSYLISHQRTH |
| ZN07_HUMAN | 1020 | YKCNKCTKAFGCSSRLIRHQRTH |
| Z263_HUMAN | 1021 | YQCNICGKCFSCNSNLHRHQRTH |
| Q13134_HUMAN | 1022 | YKCELCPYSSSQKTHLTRHMRTH |
| Q13127_HUMAN | 1023 | YKCELCPYSSSQKTHLTRHMRTH |
| CTCF_HUMAN | 1024 | FQCSLCSYASRDTYKLKRHMRTH |
| Q99592_HUMAN | 1025 | YTCSLCGKTFSCMYTLKRHERTH |
| Q13397_HUMAN | 1026 | YTCSLCGKTFSCMYTLKRHERTH |
| O60765_HUMAN | 1027 | YKCSLCEKTFINTSSLRKHEKNH |
| ZN74_HUMAN | 1028 | YKCSACEKAFSCSSLLSMHLRVH |
| ZN75_HUMAN | 1029 | YKCQQCDRRFRWSSDLNKHFMTH |
| Z189_HUMAN | 1030 | YQCNQCKQSFSQRRSLVKHQRIH |
| O75802_HUMAN | 1031 | YQCNQCKQSFSQRRSLVKHQRIH |
| Z186_HUMAN | 1032 | YACNCCEKLFSYKSSLTIHQRIH |
| Z186_HUMAN | 1033 | YACDHCEKAFSHKSXLTVHQRTH |
| ZN84_HUMAN | 1034 | YECRDCEKAFSQKSQLNTHQRIH |
| O60792_HUMAN | 1035 | YQCNKCEKTFSQSSHLTQHQRIH |
| O75066_HUMAN | 1036 | YACQYCDAVFAQSIELSRHVRTH |
| O95878_HUMAN | 1037 | YRCDICGKSFSQSATLAVHHRTH |
| P91805_SARPE | 1038 | YQCKVCQKRFPQLSTLHNHERTH |
| Z133_HUMAN | 1039 | YACKECGRCFRQRTTLVNHQRTH |
| Z133_HUMAN | 1040 | YVCGVCGHSFSQNSTLISHRRTH |
| O43336_HUMAN | 1041 | YVCIECGKSLSSKYSLVEHQRTH |
| O75467_HUMAN | 1042 | YACAQCGRRFCRNSHLIQHERTH |
| Z124_HUMAN | 1043 | YECKQCGKAFSRSSHLRDHERTH |
| Z177_HUMAN | 1044 | YECNQCGKSFSTGSYLIVHKRTH |
| Z177_HUMAN | 1045 | YECDHCGKSFSQSSHLNVHKRTH |
| ZN84_HUMAN | 1046 | YACGNCGKTFPQKSQFITHHRTH |
| Z135_HUMAN | 1047 | YECHECGKAFTQITPLIQHQRTH |
| Z135_HUMAN | 1048 | YECNQCGRAFSQLAPLIQHQRIH |
| Z135_HUMAN | 1049 | YKCTQCGRTFNQIAPLIQHQRTH |
| O60893_HUMAN | 1050 | YQCDTCGKGFTRTSYLVQHQRSH |
| O43337_HUMAN | 1051 | YKCKQCGKGFNRKWYLVRHQRVH |
| Z205_HUMAN | 1052 | YRCEQCGKGFSWHSHLVTHRRTH |
| Z202_HUMAN | 1053 | YRCDDCGKHFRWTSDLVRHQRTH |
| ZN45_HUMAN | 1054 | YRCDVCGKRFRQRSYLQAHQRVH |
| ZN45_HUMAN | 1055 | YQCDACGKGFSRSSDFNIHFRVH |
| Z239_HUMAN | 1056 | YQCYECGKGFSQSSDLRIHLRVH |
| Z239_HUMAN | 1057 | YKCDKCGKGFSQSSKLHIHQRVH |
| Z239_HUMAN | 1058 | YHCGKCGKGFSQSSKLLIHQRVH |
| Z239_HUMAN | 1059 | YKCGECGKGFSQSSNLHIHRCIH |
| O15322_HUMAN | 1060 | YKCDMCGKEFSQSSCLQTHERVH |
| Z239_HUMAN | 1061 | YACQYCGKNFSQSSELLLHQRDH |
| ZN07_HUMAN | 1062 | YPCKECGKAFSQSSTLAQHQRMH |
| Z133_HUMAN | 1063 | YVCKTCGRGFSLKSHLSRHRKTH |
| Z133_HUMAN | 1064 | YVCGVCGRGFSLKSHLARHQNIH |
| Z133_HUMAN | 1065 | YVCGVCEKGFSLKKSLARHQKAH |
| EVI1_HUMAN | 1066 | YRCKYCDRSFSISSNLQRHVRNIH |
| RRE1_HUMAN | 1067 | YKCQTCERTFTLKHSLVRHQRIH |
| O75850_HUMAN | 1068 | YACAQCGRRFSRKSHLQRHQAVH |
| O75850_HUMAN | 1069 | HACAVCARSFSSKTNLVRHQAIH |
| O75850_HUMAN | 1070 | YQCAQCARSFTHKQHLVRHQRVH |
| ZN42_HUMAN | 1071 | FVCSECGRSFSRSSHLLRHQLTH |
| Z132_HUMAN | 1072 | FECSECGRDFSQSSHLLRHQKVH |
| ZN35_HUMAN | 1073 | YECEKCGAAFISNSHLMRHHRTH |
| Z132_HUMAN | 1074 | YECSECGRAFSSNSHLVRHQRVH |
| Z202_HUMAN | 1075 | YKCMECGKSYTRSSHLARHQKVH |
| Z134_HUMAN | 1076 | YECSECGKAYSLSSHLNRHQKVH |
| Z239_HUMAN | 1077 | YECSKCGKGFSQSSNLHSHQRVH |
| Z165_HUMAN | 1078 | YECSECGRAFSQSSNLSQHQRIH |
| Z132_HUMAN | 1079 | YECSECGRAFNNNSNLAQHQKVH |
| Z239_HUMAN | 1080 | YECEECGMSFSQRSNLHIHQRDH |
| O00153_HUMAN | 1081 | HQCQVCGKTFSQSGSRNVHMRKH |
| Q13398_HUMAN | 1082 | YVCGECGKSFSHSSNLKNHQRVH |
| O15322_HUMAN | 1083 | YKCEICGKSFCLRSSLNRHYMVH |
| O75123_HUMAN | 1084 | FKCAQCGKAFCHSSDLIRHQRVH |
| O14913_HUMAN | 1085 | YKCEECDKAFLYHSFLRRHKAVH |
| O14913_HUMAN | 1086 | YKCEECDKAFLHHSYLRKHQAVH |
| ZN83_HUMAN | 1087 | FKCNECGKLFRDNSYLVRHQRFH |
| O15322_HUMAN | 1088 | HTCNECGKSFCYISALRIHQRVH |
| O60792_HUMAN | 1089 | FGCNDCGKSFRYRSALNKHQRLH |
| Z137_HUMAN | 1090 | YKCNKCGKIFRHRSYLAVYQRTH |
| O75123_HUMAN | 1091 | YVCNVCGKDFIHYSGLIEHQRVH |
| Z134_HUMAN | 1092 | YKCNECGKYFSHHSNLIVHQRVH |
| O43361_HUMAN | 1093 | FECSICGKFFSHRSTLNMHQRVH |
| Z134_HUMAN | 1094 | FECIECGKFFSRSSDYIAHQRVH |
| Z134_HUMAN | 1095 | FVCSKCGKDFIRTSHLVRHQRVH |
| O14913_HUMAN | 1096 | YKCQECGKSFCYRSYLREHYRMH |
| Z174_HUMAN | 1097 | YKCDDCGKSFTWNSELKRHKRVH |
| O60765_HUMAN | 1098 | YRCKECGKSFSRRSGFIHQKIH |
| O43167_HUMAN | 1099 | YSCGICGKSFSDSSAKRRHCILH |
| O43829_HUMAN | 1100 | FVCEMCTKGFTTQAHLKEHLKIH |
| O00403_HUMAN | 1101 | FVCEMCTKGFTTQAHLKEHLKIH |
| O75626_HUMAN | 1102 | FKCQTCNKGFTQLAHLQKHYLVH |
| O15322_HUMAN | 1103 | FKCEQCGKGFRCRAILQVHCKLH |
| BCL6_HUMAN | 1104 | YKCETCGARFVQVAHLRAHVLIH |
| Z195_HUMAN | 1105 | YKCEKCGKAFTQFSHLTVHESIH |
| ZN85_HUMAN | 1106 | YKCKKCGKAFNQSAHLTTHEVIH |
| Z239 HUMAN | 1107 | YKCEKCGKGFTRSSSLLIHHAVH |
| Z239_HUMAN | 1108 | YKCEQCGKGFTRSSSLLIHQAVH |
| O15322_HUMAN | 1109 | YKCEECGKGFTDSLDLHKHQIIH |
| O15322_HUMAN | 1110 | YICEKCGPAFIHDLKLQKHQIIH |
| O14913_HUMAN | 1111 | YKCEKCGKGFFRSSDLQHHQKIH |
| O14913_HUMAN | 1112 | YKCEECGKCFSSFTSLKRHQIIH |
| O14913_HUMAN | 1113 | YPYKCEECGKGFSRSSKLQEHQTIH |
| ZN45_HUMAN | 1114 | YKGEHCVKSFSWSSHLQINQRAH |
| ZN45_HUMAN | 1115 | YKCEECGKGFSWSSSLIIHQRVH |
| ZN45_HUMAN | 1116 | YKCEECGKVFSWSSYLQAHQRVH |
| ZN45_HUMAN | 1117 | YKCEKCDNAFRRFSSLQAHQRVH |
| ZN45_HUMAN | 1118 | YKCERCGKAFSQFSSLQVHQRVH |
| ZN45_HUMAN | 1119 | YKCEECGVGFSQRSYLQVHLKVH |
| ZN45_HUMAN | 1120 | YKCEECGKSFSWRSRLQAHERIH |
| ZN45_HUMAN | 1121 | YKCEECGKGFSVGSHLQAHQISH |
| ZN45_HUMAN | 1122 | YQCAECGKGFSVGSQLQAHQRCH |
| ZN45_HUMAN | 1123 | YQCEECGKGFCPASNFLAHRGVH |
| ZN45_HUMAN | 1124 | YKCEECGKGFCPASNLLDHQRGH |
| ZN45_HUMAN | 1125 | YKCEECGKGFSQASNLLAHQRGH |
| O75467_HUMAN | 1126 | FVCALCGAAFSQGSSLFKHQRVH |
| ZN42_HUMAN | 1127 | YHCGECGLGFTQVSRLTEHQRIH |
| O60765_HUMAN | 1128 | YRCNECGKGFTSISRLNRHRIIH |
| TYY1_HUMAN | 1129 | YVCPFDGCNKKFAQSTNLKSHILTH |
| O15391_HUMAN | 1130 | FVCPFDVCNRKFAQSTNLKTHILTH |
| TYY1_HUMAN | 1131 | FQCTFEGCGKRFSLDFNLRTHVRIH |
| O15391_HUMAN | 1132 | FQCTFEGCGKRFSLDFNLRTHLRIH |
| Q14872_HUMAN | 1133 | YQCTFEGCPRTYSTAGNLRTHQKTH |
| GLI1_HUMAN | 1134 | HKCTFEGCRKSYSRLENLKTHLRSH |
| GLI3_HUMAN | 1135 | HKCTFEGCTKAYSRLENLKTHLRSH |
| O60255_HUMAN | 1136 | HKCTFEGCSKAYSRLENLKTHLRSH |
| O60254_HUMAN | 1137 | HKCTFEGCSKAYSRLENLKTHLRSH |
| O60253_HUMAN | 1138 | HKCTFEGCSKAYSRLENLKTHLRSH |
| O60252_HUMAN | 1139 | HKCTFEGCSKAYSRLENLKTHLRSH |
| GLI2_HUMAN | 1140 | HKCTFEGCSKAYSRLENLKTHLRSH |
| O95409_HUMAN | 1141 | FQCEFEGCDRRFANSSDRKKHMHVH |
| Q15915_HUMAN | 1142 | FKCEFEGCDRRFANSSDRKKHMHVH |
| ZIC3_HUMAN | 1143 | FKCEFEGCDRRFANSSDRKKHMHVH |
| GLI1_HUMAN | 1144 | YMCEHEGCSKAFSNASDRAKHQNRTH |
| O60255_HUMAN | 1145 | YVCEHEGCNKAFSNASDRAKHQNRTH |
| O60254_HUMAN | 1146 | YVCEHEGCNKAFSNASDRAKHQNRTH |
| O60253_HUMAN | 1147 | YVCEHEGCNKAFSNASDRAKHQNRTH |
| O60252_HUMAN | 1148 | YVCEHEGCNKAFSNASDRAKHQNRTH |
| GLI3_HUMAN | 1149 | YVCEHEGCNKAFSNASDRAKHQNRTH |
| GLI2_HUMAN | 1150 | YVCEHEGCNKAFSNASDRAKHQNRTH |
| Z143_HUMAN | 1151 | YVCTVPGCDKRFTEYSSLYKHHVVH |
| TF3A_HUMAN | 1152 | FKCTQEGCGKHFASPSKLKRHAKAH |
| TF3A_HUMAN | 1153 | FVCDYEGCGKAFIRDYHLSRHILTH |
| Q14872_HUMAN | 1154 | FECDVQGCEKAFNTLYRLKAHQRLH |
| Q14872_HUMAN | 1155 | FVCNQEGCGKAFLTSHSLRIHVRVH |
| ZN76_HUMAN | 1156 | YRCDFPSCGKAFATGYGLKSHVRTH |
| Z143_HUMAN | 1157 | YQCEHAGCGKAFATGYGLKSHVRTH |
| Q14872_HUMAN | 1158 | FRCDHDGCGKAFAASHHLKTHVRTH |
| O00153_HUMAN | 1159 | FICPAEGCGKSFYVLQRLKVHMRTH |
| ZN76_HUMAN | 1160 | FQCPFEGCGRSFTTSNIRKVHVRTH |
| Z143_HUMAN | 1161 | FKCPFEGCGRSFTTSNIRKVHVRTH |
| Q15915_HUMAN | 1162 | FPCPFPGCGKVFARSENLKIHKRTH |
| O95409_HUMAN | 1163 | FPCPFPGCGKVFARSENLKIHKRTH |
| ZIC3_HUMAN | 1164 | FPCPFPGCGKIFARSENLKIHKRTH |
| ZN76_HUMAN | 1165 | YTCPEPHCGRGFTSATNYKNHVRIH |
| Z143_HUMAN | 1166 | YYCTEPGCGPAFASATNYKNHVRIH |
| O00153_HUMAN | 1167 | FMCHESGCGKQFTTAGNLKNHRRIH |
| ZN76_HUMAN | 1168 | YKCPEELCSKAFKTSGDLQKHVRTH |
| Z143_HUMAN | 1169 | YRCSEDNCTKSFKTSGDLQKHIRTH |
| Q14872_HUMAN | 1170 | FNCESEGCSKYFTTLSDLRKHIRTH |
| ZN76_HUMAN | 1171 | FRCGYKGCGRLYTTAHHLKVHERAH |
| Z143_HUMAN | 1172 | FRCEYDGCGKLYTTARHLKVHERSH |
| BTE1_HUMAN | 1173 | HKCPYSGCGKVYGKSSHLKAHYRVH |
| BTE2_HUMAN | 1174 | HYCDYPGCTKVYTKSSHLKAHLRTH |
| O43839_HUMAN | 1175 | HRCHFNGCRKVYTKSSHLKAHQRTH |
| UKLF_HUMAN | 1176 | HRCQFNGCRKVYTKSSHLKAHQRTH |
| O95600_HUMAN | 1177 | HQCDFAGCSKVYTKSSHLKAHRRIH |
| Q13118_HUMAN | 1178 | HICSHPGCGKTYFKSSHLKAHTRTH |
| O75411_HUMAN | 1179 | HICSHPGCGKTYFKSSHLKAHTRTH |
| EZF_HUMAN | 1180 | HTCDYAGCGKTYTKSSHLKAHLRTH |
| O14901_HUMAN | 1181 | YVCSFPGCRKTYFKSSHLKAHLRTH |
| SP4_HUMAN | 1182 | HICHIEGCGKVYGKTSHLRAHLRWH |
| O60402_HUMAN | 1183 | HICHIEGCGKVYGKTSHLRAHLRWH |
| EKLF_HUMAN | 1184 | HTCAHPGCGKSYTKSSHLKAHLRTH |
| WT1_HUMAN | 1185 | FMCAYPGCNKRYFKLSHLQMHSRKH |
| Q16256_HUMAN | 1186 | FMCAYPGCNKRYFKLSHLQMHSRKH |
| Q15881_HUMAN | 1187 | FMCAYPGCNKRYFKLSHLQMHSRKH |
| SP2_HUMAN | 1188 | HVCHIPDCGKTFRKTSLLRAHVRLH |
| O43167_HUMAN | 1189 | YACKDCHRKFMDVSQLKKHLRTH |
| O75467_HUMAN | 1190 | YACRACSKVFVKSSDLLKHLRTH |
| ZEP1_HUMAN | 1191 | YICEYCNRACAKPSVLLKHIRSH |
| Q02646_HUMAN | 1192 | YICPYCSRACAKPSVLKKHIRSH |
| O75362_HUMAN | 1193 | YACSYCGKFFRSNYYLNIHLRTH |
| Q92981_HUMAN | 1194 | YKCVQPDCGKAFVSRYKLMRHMATH |
| O76019_HUMAN | 1195 | YKCVQPDCGKAFVSRYKLMRHMATH |
| RRE1_HUMAN | 1196 | YACSVCNKRFWSLQDLTRHMRSH |
| O75626_HUMAN | 1197 | HECQVCHKRFSSTSNLKTHLRLH |
| Z202_HUMAN | 1198 | HDCSVCGKSFTCNSHLVRHLRTH |
| O75123_HUMAN | 1199 | YACDICGKTFTFNSDLVRHRISH |
| Z151_HUMAN | 1200 | HKCSVCSKAFVNVGDLSKHIIIH |
| SNAI_HUMAN | 1201 | YACVCGTCGKAFSRPWLLQGHVRTH |
| O43623_HUMAN | 1202 | YACVCKICGKAFSRPWLLQGHIRTH |
| O95409_HUMAN | 1203 | HVCFWEECPREGKPFKAKYKLVNHIRVH |
| ZIC3_HUMAN | 1204 | HVCYWEECPREGKSFKAKYKLVNHIRVH |
| O00146_HUMAN | 1205 | HECKLCGASFRTKGSLIRHHRRH |
| O00146_HUMAN | 1206 | HVCQFCSRGFREKGSLVRHVRHH |
| IKAR_HUMAN | 1207 | FQCNQCGASFTQKGNLLRHIKLH |
| CTCF_HUMAN | 1208 | HKCHLCGRAFRTVTLLRNHLNTH |
| HKR3_HUMAN | 1209 | HVCEFCSHAFTQKANLNMHLRTH |
| Q15552_HUMAN | 1210 | HVCEHCNAAFRTNYHLQRHVFIH |
| O43591_HUMAN | 1211 | HVCEHCNAAFRTNYHLQRHVFIH |
| PLZF_HUMAN | 1212 | YICSECNRTFPSHTALKRHLRSH |
| Z151_HUMAN | 1213 | YVCIHCQRQFADPGALQRHVRIH |
| MAZ_HUMAN | 1214 | YICALCAKEFKNGYNLRRHEAIH |
| O14753_HUMAN | 1215 | HLCTGCGKGFNDTFDLKRHVRTH |
| O95365_HUMAN | 1216 | YECNICKVRFTRQDKLKVHMRKH |
| O15156_HUMAN | 1217 | YACEVCGVRFTRNDKLKIHMRKH |
| O75066_HUMAN | 1218 | YSCEECGAKFAANSTLKNHLRLH |
| O95365_HUMAN | 1219 | YLCQQCGAAFAHNYDLKNHMRVH |
| O15156_HUMAN | 1220 | YSCPHCPARFLHSYDLKNHMHLH |
| Z151_HUMAN | 1221 | HKCEDCGKEFTHTGNFKRHIRIH |
| Z151_HUMAN | 1222 | YRCEDCGKLFTTSGNLKRHQLVH |
| Z151_HUMAN | 1223 | YKCRECGKQFTTSGNLKRHLRIH |
| O15090_HUMAN | 1224 | YDCPYCGKTFRTSHHLKVHLRIH |

### Example 3

### Non-Human Zinc Finger Databases

For providing novel combinations of non-antigenic, optimised zinc fingers, for use in species other than humans, separate species-specific zinc finger databases are required, such as mouse, chicken, pig, cow, etc.

The fingers listed below are in a format that can be linked with classical wild-type canonical “TGEKP” linkers (i.e . . . TGEKP— zinc finger peptide sequence—TGEKP—zinc finger peptide sequence -TGEKP—etc . . . ). For each peptide sequence, an oligonucleotide is designed to encode the peptide sequence; the oligonucleotide can then be linked into a library selection system, as described in the Examples infra.

Mouse Zinc Finger Database.

| 544 zinc | | |
|---|---|---|
| finger units | SEQ | |
| Name | ID NO | Peptide sequence |
| O35745_MOUSE | 1225 | HQCTHCEKTFNRKDHLKNHLQTH |
| ZFX2_MOUSE | 1226 | HRCEYCKKGFRRPSEKNQHIMRH |
| ZFX1_MOUSE | 1227 | HRCEYCKKGFRRPSEKNQHIMRH |
| ZFY2_MOUSE | 1228 | HKCDMCSKGFHRPSELKKHVATH |
| ZFY1_MOUSE | 1229 | HKCDMCSKGFHRPSELKKHVATH |
| ZFX2_MOUSE | 1230 | HKCDMCDKGFHRPSELKKWVAAH |
| ZFX1_MOUSE | 1231 | HKCDMCDKGFHRPSELKKHVAAH |
| ZFA_MOUSE | 1232 | HKCDMCDKGFHRPSELKKHVAAH |
| Q9Z162_MOUSE | 1233 | YTCSVCGKGFSRPDHLSCHVKHVH |
| MAZ_MOUSE | 1234 | YNCSHCGKSFSRPDHLNSHVRQVH |
| Q08376_MOUSE | 1235 | YSCEVCGKSFIRAPDLKKHERVH |
| Z151_MOUSE | 1236 | HKCPHCDKKFNQVGNLKAHLKIH |
| ZFX2_MOUSE | 1237 | FRCKRCRKGFRQQSELKKHMKTH |
| ZFX1_MOUSE | 1238 | FRCKRCRKGFRQQSELKKHMKTH |
| Q62518_MOUSE | 1239 | YVCTMCGKGYTLNSNLQVHLRVH |
| Q60636_MOUSE | 1240 | YECNVCAKTFGQLSNLKVHLRVH |
| Q9Z117_MOUSE | 1241 | CSCPECGKVLHQLSHLRSHYRLH |
| Q61898_MOUSE | 1242 | CSCPECGREFHQLSHLRKHYRLH |
| Q88631_MOUSE | 1243 | YSCQYCGKVFHQLSHFKSHFTLH |
| Q61164_MOUSE | 1244 | HKCPDCDMAFVTSGELVRHRRYKH |
| O35483_MOUSE | 1245 | FRCADCGRGFAQRSNLAKHRRGH |
| O35483_MOUSE | 1246 | FVCGVCGAGFSRRAHLTAHGRAH |
| O70162_MOUSE | 1247 | FVCRDCGQGFVRSARLEEHRRVH |
| Q9Z1D8_MOUSE | 1248 | HRCGDCGKFFLQASNFIQHRRIH |
| O35483_MOUSE | 1249 | HRCPDCGKGFGHSSDFKRHRRTH |
| O35483_MOUSE | 1250 | ---ADCGKSFVYGSHLARHRRTH |
| O35483_MOUSE | 1251 | FPCPDCGKRFVYKSHLVTHRRIH |
| O88282_MOUSE | 1252 | YKCQLCRSAFRYKGNLASHRTVH |
| Q61065_MOUSE | 1253 | YKCDRCQASFRYKGNLASHKTVH |
| BCL6_MOUSE | 1254 | YKCDRCQASFRYKGNLASHKTVH |
| O70162_MOUSE | 1255 | FACQDCGRRFNQSTKLIQHQRVH |
| O70162_MOUSE | 1256 | --CVECGERFGRRSVLLQHRRVH |
| Q9Z0G7_MOUSE | 1257 | -DCPVCNKKFKMKHHLTEHMKTH |
| Q08376_MOUSE | 1258 | ---HMCDKAFKHKSHLKDHERRH |
| Q64318_MOUSE | 1259 | HECGICRKAFKHKHHLIEHMRLH |
| Q64318_MOUSE | 1260 | FKCTECGKAFKYKHHLKEHLRIH |
| Q9Z1D8_MOUSE | 1261 | FKCNECGKGFGRRSHLAGHLRLH |
| Q9Z1D8_MOUSE | 1262 | YGCNECGKSFGRHSHLIEHLKRH |
| Q9Z2X6_MOUSE | 1263 | ----YVCKQCGKAFTLSSSLRRH |
| KID1_MOUSE | 1264 | YVCKECGKAFTLSTSLYKHLRTH |
| Q9Z1D7_MOUSE | 1265 | HGCDECGKSFTQHSRLIEHKRVH |
| ZF90_MOUSE | 1266 | YRCNLCGRSFRHSTSLTQHEVTH |
| Q9Z2X6_MOUSE | 1267 | YVCKECGKAFARSTSLHIHEGTH |
| Q9Z2X6_MOUSE | 1268 | YVCKHCGKAYTTYNTLRAHERSH |
| Q9Z2X6_MOUSE | 1269 | YVCKHCGKAYTTYNTLRAHERSH |
| Q9Z2X6_MOUSE | 1270 | YVCKHCGKAYTSYSTLRAHERSH |
| Q9Z2X6_MOUSE | 1271 | YVCKHCGKAYTSYSTLRAHERSH |
| Q9Z2X6_MOUSE | 1272 | YVCKHCGKAYTSYSTLRARERSH |
| Q9Z2X6_MOUSE | 1273 | YVCKHCGKAFTQSSYLRIHKRTH |
| ZF37_MOUSE | 1274 | YECEQCGKAHGHKHALTDHLRIH |
| Q62514_MOUSE | 1275 | YECEQCGKAHGHKHALTDHLRIH |
| Q61491_MOUSE | 1276 | YECNQCGKAFTQFFPLKRHEITH |
| ZF37_MOUSE | 1277 | YKCDECGKAFGHSSSLTYHMRTH |
| Q62514_MOUSE | 1278 | YKCDECGKAFGHSSSLTYHMRTH |
| Q61491_MOUSE | 1279 | YQCNQCAKAFPYHRTLQIHERTH |
| Q61491_MOUSE | 1280 | CEYNQCWKAFAYHKTLQIHERTH |
| Q61491_MOUSE | 1281 | YECNQCGKAFACYQSFQIHKRTH |
| Q61491_MOUSE | 1282 | YECNQCGKAFACNRYLQIHKRTH |
| Q61491_MOUSE | 1283 | YECNQCGKAFACPRYLQIHKRTH |
| Q61491_MOUSE | 1284 | YECNQCGKAFACLRNLQNHKTTH |
| Q61491_MOUSE | 1285 | FECNQCGKAFAHHSTLQRHKRTH |
| Q61491_MOUSE | 1286 | YECNQCGKAFTRHSTLQIHKRTH |
| Q61491_MOUSE | 1287 | YECNQCGKAFTCRSNLQIHKRTH |
| Q9Z2X6_MOUSE | 1288 | YVCKQCGKAFTRSSHLQIHKITH |
| Q9Z2X6_MOUSE | 1289 | YICKQCGKAFARSSHLQIHKRSH |
| Q61491_MOUSE | 1290 | YKCKQCGKDFTHHSTLHIHKRIH |
| Q9Z2X6_MOUSE | 1291 | YSCKLCGKAFTHSNYLQIHKRIH |
| Q61491_MOUSE | 1292 | YECNQCGKAFARNSNLLDHKRIH |
| Q64247_MOUSE | 1293 | YICKQCGKTFRYLSCFQKHERIH |
| Q9Z2X6_MOUSE | 1294 | YACKQCDKAFKYLSSLQNHKRIH |
| Q9Z2X6_MOUSE | 1295 | HACKQCGKSFKRQSNVQAHERNH |
| Q64247_MOUSE | 1296 | YTCKHCTKTETTSSTRNSHEKTH |
| Q64247_MOUSE | 1297 | YACKHCGKAFTTSSARNSHERIH |
| Q64247_MOUSE | 1298 | YACKHCGKAFTSSSDRNSHERIH |
| Q64247_MOUSE | 1299 | YPCKYCGKAFATSSDRNSHERIH |
| Q64247_MOUSE | 1300 | YSCTHCGKAFSSPSDYNSCERIH |
| Q88412_MOUSE | 1301 | YVCNECGKAFTCSSYLLIHQRIH |
| ZF35_MOUSE | 1302 | YMCNHCYKHFSQSSDLIKHQRIH |
| Q9Z2X6_MOUSE | 1303 | YVCKQCGKAFAQSSYLHIHQRSH |
| ZF38_MOUSE | 1304 | YQCKDCGKAFSGKGSLIRHYRIH |
| OZF_NOUSE | 1305 | YECNKCGKAFSRITSLIVHVRIH |
| Q9Z0Q5_MOUSE | 1306 | YECNECGKAFSQRTSLIVHVRIH |
| ZF90_MOUSE | 1307 | YQCNVCGKAFKRSTSFIEHHRIH |
| OZF_MOUSE | 1308 | YECKICGKAFCQSSSLTVHMRSH |
| Q9Z0Q5_MOUSE | 1309 | YECNVCGKAFSQSSSLTVHVRSH |
| ZF90_MOUSE | 1310 | YECIDCGKAFSQSSSLIQHERTH |
| Z151_MOUSE | 1311 | CQCVICGKAFTQASSLIAHVRQH |
| OZF_MOUSE | 1312 | YECKGCGKAFIQKSSLIRHQRSH |
| Q9Z0Q5_MOUSE | 1313 | FECKDCGKAFIQKSNLIRHQRTH |
| Q9Z162_MOUSE | 1314 | ---TYCSKAFRDSYHLRRHQSCH |
| Q9Z162_MOUSE | 1315 | HACEMCGKAFRDVYHLNRHKLSH |
| MAZ_MOUSE | 1316 | HACEMCGKAFRDVYHLNRHKLSH |
| Q61898_MOUSE | 1317 | FRCTECDKSFIRSSHLREHQKIH |
| Q60585_MOUSE | 1318 | FDCKECGKTFSRGYHLTLHQRIH |
| Q35483_MOUSE | 1319 | YACAECGRRFGQSAALTRHQWAH |
| Q60585_MOUSE | 1320 | YACTECGKSFRQVAHLTRHQRLN |
| Q9Z1D9_MOUSE | 1321 | YACPECGECFRQSSHLSRHQRTH |
| Q9Z1D9_MOUSE | 1322 | YKCFQCGERFRQSTHLVRHQRIH |
| Q88631_MOUSE | 1323 | YKCTKCDKLFTQYSHLRRHQRIY |
| Q60585_MOUSE | 1324 | YKCTECKKAFRQHSHLTYHQRIH |
| MLZ4_MOUSE | 1325 | HKCTECAKASAASPHLIQHQRTH |
| Q9Z116_MOUSE | 1326 | YECTECSKAFCQKSHLTQHQRVH |
| Q70237_MOUSE | 1327 | YPCQFCGKRFHQKSDMKKHTYIH |
| GFI1_MOUSE | 1328 | YPCQYCGKRFHQKSDMKKHTFIH |
| Q61624_MOUSE | 1329 | FRCDECGMRFIQKYHMERHKRTH |
| P97475_MOUSE | 1330 | PRCDECGMRFIQKYHMERHKRTH |
| Q61624_MOUSE | 1331 | FQCSQCDMRFIQKYLLQRHEKIH |
| P97475_MOUSE | 1332 | FQCSQCDMRFIQKYLLQRHEKIH |
| ZFP1_MOUSE | 1333 | FVCNYCDKTFSFKSLLVSHKRIH |
| Q9Z116_MOUSE | 1334 | YICFECRKAFYRKSELTDHQRIH |
| Q9Z116_MOUSE | 1335 | YECKECGKAFCQKPQLTLHQRIH |
| ZFP1_MOUSE | 1336 | YGCSECGKTFAQKFELTTHQRIH |
| Q06054_MOUSE | 1337 | YKCSDCGKCFIQKANLRTHQKIH |
| Q06054_MOUSE | 1338 | YKCSDCGKCFIQKANLRTHERIH |
| Q06054_MOUSE | 1339 | YKCSDCDKCFIQKAKLKKHQRIH |
| Q06054_MOUSE | 1340 | YKCSECDKCFIQKDHLRTHQRLH |
| Q06054_MOUSE | 1341 | YKCSECDKCFIRKANLRRHHRIH |
| Q06054_MOUSE | 1342 | YKCSECHKCFIRKAHLRRHQRIH |
| Q06054_MOUSE | 1343 | YKCSECHKCFIQQAHLRRHQKIH |
| Q06054_MOUSE | 1344 | YICAECNKCFIQKSQLKTHQRIH |
| MLZ4_MOUSE | 1345 | HICSQCGKAFSQISDLNRHQKTH |
| ZF37_MOUSE | 1346 | YECNECGIAFSQKSHLVVHQRTH |
| Q62514_MOUSE | 1347 | YECNECGIAFSQKSHLVLHQRTH |
| ZF37_MOUSE | 1348 | YECVECGKAFSQKSHLIVHQRPH |
| Q62514_MOUSE | 1349 | YECVECGKAFSQKSHLIVHQRTH |
| ZF37_MOUSE | 1350 | FECNECGKTFSKKSHLVIHQRTH |
| Q62514_MOUSE | 1351 | FECNECGKTFSKKSHLNIHQRTH |
| MFG3_MOUSE | 1352 | FECKECGKAFHFSSQLNNHKTSH |
| Q62514_MOUSE | 1353 | FECYECGKAFNAKSQLVIHQRSH |
| ZF37_MOUSE | 1354 | FECYECGKAFNAKSQLVIHQRSH |
| Q9Z116_MOUSE | 1355 | YECKICGKCFYWKTSFNRHQSTH |
| O88412_MOUSE | 1356 | YSCNECGKAFRQKSSLTVHQRTH |
| Q9Z116_MOUSE | 1357 | YECAECGKAFSTKSYLTVHQRTH |
| P70405_MOUSE | 1358 | YECSKCGKTFRGKYSLDQHQRVH |
| ZF90_MOUSE | 1359 | HECADCGKTFLWRTQLTEHQRIH |
| KR2_MOUSE | 1360 | YECMICGKHFTGRSSLTVHQVTH |
| KR2_MOUSE | 1361 | YECDQCGKAFIKNSSLIVHQRIH |
| Q9Z1D7_MOUSE | 1362 | YKCSVCGKAFIQKISLIEHEQIH |
| Q61116_MOUSE | 1363 | YKCDTCGKAFSQKSSLQVHQRIH |
| O70237_MOUSE | 1364 | --CRMCGKAFKRSSTLSTHLLIH |
| GFI1_MOUSE | 1365 | -DCKICGKSFKRSSTLSTHLLIH |
| Q9Z150_MOUSE | 1366 | HSCGICGKCFTQKSTLHDHLNLH |
| Q9Z1D7_MOUSE | 1367 | YKCEVCGKTFRWRTVLIRHKVVH |
| ZF35_MOUSE | 1368 | -YKCMCGKAFSQCSAFTLHQRIH |
| ZF38_MOUSE | 1369 | YKCKECGKAFNHSSNFNKHHRIH |
| OZF_MOUSE | 1370 | YGCNECGKAFSQFSTLALHMRIH |
| Q9Z0Q5_MOUSE | 1371 | YGCNECGKAFSQFSTLALHLRIH |
| ZFP1_MOUSE | 1372 | YECTECGKTFSQRSTLRLHLRIH |
| MLZ4_MOUSE | 1373 | YKCDECGKNFSQNSDLVRHRRAH |
| Q62514_MOUSE | 1374 | YECNECGKAFKYGSSLTKHMRIH |
| ZF37_MOUSE | 1375 | YECNECGKAFKYGSSLTKHMRIH |
| KR2_MOUSE | 1376 | YKCHDCGKAFSKNSSLTQHRRIH |
| P70405_MOUSE | 1377 | CRDCGKFFSQTSHLWDHRRIHTG |
| Q61117_MOUSE | 1378 | YKCSTCGKGFSRSSDLNVHCRIH |
| ZF92_MOUSE | 1379 | YLCQQCGKSPSRSFNLIKHRIIH |
| ZF29_MOUSE | 1380 | YACKECGESFSYNSNLIRHQRIH |
| O88282_MOUSE | 1381 | YRCSICGARFNRPANLKTHSRIH |
| Q61065_MOUSE | 1382 | YRCNICGAQFNRPANLKTHTRIH |
| BCL6_MOUSE | 1383 | YRCNICGAQFNRPANLKTHTRIH |
| ZF29_MOUSE | 1384 | YKCRDCGKSFSRSANLITHQRIH |
| Q9Z1D7_MOUSE | 1385 | YQCLQCNKSFNRRSTLSQHQGVH |
| ZF35_MOUSE | 1386 | YPCNSCSKSFSRGSDLIKHQRVH |
| ZF35_MOUSE | 1387 | YPCSWCIKSFSRSSDLIKHQRVH |
| ZF35_MOUSE | 1388 | YPCNQCTKSFSRLSDLINHQRIH |
| ZFP1_MOUSE | 1389 | YECDVCQKTFSHKANLIKHQRIH |
| ZF35_MOUSE | 1390 | YECDKCGKTFSQSSNLILHQRIH |
| O88412_MOUSE | 1391 | YECNECGKTFTRSSNLIVHQRIH |
| MLZ4_MOUSE | 1392 | YDCNECGKSFGRSSHLIQHQTIH |
| MLZ4_MOUSE | 1393 | YECTACGKSFSRSSHLITHQKIH |
| KR2_MOUSE | 1394 | YECTECGKAFSQSAALIEHRRIH |
| ZF90_MOUSE | 1395 | YACKECGRNPSRSSALTKHHRVH |
| MLZ4_MOUSE | 1396 | YECTECDKSFSRSSALIKHKRVH |
| P70405_MOUSE | 1397 | YKCSECGKSFSQSSILIQHRRIH |
| P70405_MOUSE | 1398 | YKCSECGNSFSQSAILNQHRRIH |
| Q9Z1D8_MOUSE | 1399 | HQCNECGKSFIQSAHLIQHRRIH |
| KID1_MOUSE | 1400 | YRCQECGMSFGQSSALIQHRRIH |
| P70405_MOUSE | 1401 | YECSQCGKSFSQKSGLIQHQVVH |
| P70405_MOUSE | 1402 | YECRECGKSFSQKATLIKHQRVH |
| P70405_MOUSE | 1403 | YECSQCGKSFSQKATLVKHKRVH |
| Q9Z1D8_MOUSE | 1404 | HQCNECGRGFSLKSHLSQHQRIH |
| OZF_MOUSE | 1405 | YQCSECGKAFSQKSHHIRHQRIH |
| Q9Z0Q5_MOUSE | 1406 | YQCSECGKAFSQKSHHIRHQKIH |
| O88412_MOUSE | 1407 | YDCSECGKAFSQLSCLIVHQRIH |
| ZF35_MOUSE | 1408 | YKCSECGKAFNQSSVLILHQRIH |
| ZF35_MOUSE | 1409 | YKCDVCGKAFSQSSDRILHQRIH |
| KID1_MOUSE | 1410 | FKCNTCGKTFRQSSSRIAHQRIH |
| OZF_MOUSE | 1411 | YKCNECGTIFRQKQYLIKHHNIH |
| Q9Z0Q5_MOUSE | 1412 | FKCNECGTAFGQKKYLIKHQNIH |
| OZF_MOUSE | 1413 | FECSQCGRAFSQKQYLIKHQNIH |
| Q9Z0Q5_MOUSE | 1414 | FECNECGKAFSQKQYVIKHQSTH |
| OZF_MOUSE | 1415 | FKCNECGKAFSQKENLIIHQRIH |
| Q9Z0Q5_MOUSE | 1416 | FECSDCGKAFSQKENLLTHQKIH |
| KID1_MOUSE | 1417 | FKCSECGRAFSQSASLIQHERIH |
| O88412_MOUSE | 1418 | FECHECGKAFIQSANLVVHQRIH |
| O88412_MOUSE | 1419 | FTCSECGKGFSQSANLVVHQRIH |
| O88412_MOUSE | 1420 | FACSDCGKAFTQSANLIVHQRSH |
| KR2_MOUSE | 1421 | YKCHECGKAFSQSMNLTVHQRTH |
| ZF38_MOUSE | 1422 | YQCNECGKSFSQHAGLSSHQRLH |
| KID1_MOUSE | 1423 | YNCNECGKALSSHSTLIIHERIH |
| O35700_MOUSE | 1424 | YKCDQCPKAFNWKSNLIRHQMSH |
| EVI1_MOUSE | 1425 | YKCDQCPKAFNWKSNLIRHQMSH |
| Q62518_MOUSE | 1426 | YKCDVCGKSFGWRSNLIIHHRIH |
| Q9Z1D8_MOUSE | 1427 | YACHLCGKAFRVRSHLVQHQSVH |
| Q9Z1D8_MOUSE | 1428 | YKCQVCGKAFRVSSHLVQHHSVH |
| Q9Z1D7_MOUSE | 1429 | YECNDCGKAFVYNSSLATHQETH |
| MFG3_MOUSE | 1430 | YKCNACGRAFNRRSNLMQHEKIH |
| MFG3_MOUSE | 1431 | YKCNVCGKAFNRRSNLLQHQKIH |
| O88412_MOUSE | 1432 | YVCGKCGKAFTQSSNLTVHQKIH |
| Q9Z116_MOUSE | 1433 | YECKECRKAFYDKSNLKRHQKIH |
| Q60585_MOUSE | 1434 | YECKECRKFFRRYSELISHQGIH |
| Q60585_MOUSE | 1435 | YECKECGKAFRQCAHLSRHQRIH |
| ZF37_MOUSE | 1436 | YECIECGKAFKQNASLTKHMKIH |
| Q62514_MOUSE | 1437 | YECIECGKAFKQNASLTKHMKIH |
| Q61849_MOUSE | 1438 | YECNECGKAFKRHRSFVRHQKIH |
| MFG3_MOUSE | 1439 | FECKDCGKVFRLNIHLIRHQRFH |
| Q61849_MOUSE | 1440 | YECKECGKAFRLPQQLTRHQKCH |
| Q06054_MOUSE | 1441 | HRCNECGKSLSSSSGLQRHQRIH |
| O35700_MOUSE | 1442 | HACPECGKTFATSSGLKQHKHIH |
| EVI1_MOUSE | 1443 | HACPECGKTFATSSGLKQHKHIH |
| ZF92_MOUSE | 1444 | YECGECGKTFTRSSNLVKHQVIH |
| O88412_MOUSE | 1445 | FKCSECEKAFSYSSQLARHQKVH |
| ZF90_MOUSE | 1446 | FECNVCGKAFRHSSSLGQHENAH |
| KID1_MOUSE | 1447 | YECNTCGKLFNHRSSLTNHYKIH |
| ZF29_MOUSE | 1448 | YKCDECGKSFSDGSNFSRHQTTH |
| OZF_MOUSE | 1449 | YKCGECGKAFSQRGNFLSHQKQH |
| O70162_MOUSE | 1450 | CDVCGKVFSQRSNLLRHQKIHTG |
| ZFP1_MOUSE | 1451 | YECNECAKTFFKKSNLIIHQKIH |
| O88412_MOUSE | 1452 | YKCKDCEKAFSCFSHLIVHQRIH |
| Q9Z1D7_MOUSE | 1453 | YKCNECGRAFGQWSALNQHQRLH |
| ZF90_MOUSE | 1454 | YQCSLCGKAFQRSSSLVQHQRIH |
| Q64247_MOUSE | 1455 | -----CGKVFILSGDLIKHERIH |
| MFG3_MOUSE | 1456 | YECEQCGSAFRLPYQLTQHQRIH |
| Q61849_MOUSE | 1457 | FECELCGSAFRCRSQLNKHLRIH |
| MFG3_MOUSE | 1458 | FKCKLCESAFRRKYQLSEHQRIH |
| Q61849_MOUSE | 1459 | FKCQECGKAFVVLAYLIEHQSIH |
| Q64247_MOUSE | 1460 | FVCKQCGEAFVNSSHLISHERIH |
| MFG3_MOUSE | 1461 | FQCKECGRAFVRSTGLRIHERIH |
| Q64247_MOUSE | 1462 | FVCKTCGKAFSRSDYLINHKRIH |
| Q64247_MOUSE | 1463 | FVCKKCGKAFKRLGHFMNHERIH |
| ZF90_MOUSE | 1464 | FQCKECGKAFSRCSSLVQHERTH |
| MFG3_MOUSE | 1465 | FECKDCGKAFTVLAQLTRHQTIH |
| MFG3_MOUSE | 1466 | FHCKVCGKAFTVLAQLTRHENIH |
| MFG3_MOUSE | 1467 | FECKECGKSFKRVSSLVEHRIIH |
| ZFP1_MOUSE | 1468 | FECPECGKAFTHQSNLIVHQRAH |
| ZF92_MOUSE | 1469 | FECTECGKAFSRSSNLIEHQRIH |
| O54978_MOUSE | 1470 | FECQECGEAFARRSELIEHQKIH |
| O70162_MOUSE | 1471 | FRCTECGQSFRQRSNLLQHQRIH |
| O70162_MOUSE | 1472 | FACAECGQSFRQRSNLTQHQRIH |
| O70162_MOUSE | 1473 | FACPECGQSFRQHANLTQHRRIH |
| O70162_MOUSE | 1474 | YACAECGKAFRQRPTLTQHLRTH |
| O70162_MOUSE | 1475 | AECGKTFRQRATLTQHLCVHTGE |
| Q9Z1D8_MOUSE | 1476 | FRCEECGKSYNQRVHLIQHHRVH |
| Q9Z1D8_MOUSE | 1477 | FKCGECGKSYNQRVHLTQHQRVH |
| ZF37_MOUSE | 1478 | FECNQCGKAFKQIEGLTQHQRVH |
| Q62514_MOUSE | 1479 | FECNQCGKAFKQIEGLTQHQRVH |
| O88282_MOUSE | 1480 | YPCPTCGTRFRHLQTLKSHVRIH |
| Q61065_MOUSE | 1481 | YPCEICGTRFRHLQTLKSHLRIH |
| BCL6_MOUSE | 1482 | YPCEICGTRFRRLQTLKSHIRIH |
| Q60585_MOUSE | 1483 | YDCKECGKAFRVRQQLTLHERIH |
| Q60585_MOUSE | 1484 | YDCKECGKAFRVRGQLMLHQRIH |
| Q60585_MOUSE | 1485 | YECGECGKAFKVRQQLTFHQRIH |
| OZF_MOUSE | 1486 | YACKECGKAFNGKSYLKEHEKIH |
| OZF_MOUSE | 1487 | YTCKECGKAFSGKSNLTEHEKIH |
| Q9Z0Q5_MOUSE | 1488 | FICKECGKTFSGKSNLTEHEKIH |
| MFG3_MOUSE | 1489 | YKCKDCGKCFGCKSNLHQHESIH |
| Q61849_MOUSE | 1490 | YQCKECGKCFRQRSKLTEHESIH |
| Q61849_MOUSE | 1491 | YECKECGKCFGCRSTLTQHQSVH |
| Q61849_MOUSE | 1492 | FECEECGKKFRTARHLVKHQRIH |
| ZF92_MOUSE | 1493 | FVCRMCGKVFRRSFALLEHTRIH |
| ZF92_MOUSE | 1494 | YECSECGKQFQRSLALLEHQRIH |
| ZF35_MOUSE | 1495 | YECEECGKAFRMSSALVLHQRIH |
| P70405_MOUSE | 1496 | YECSECGKLFRQNSSLVDHQKTH |
| REX1_MOUSE | 1497 | HVCAECGKAFTESSKLKRHFLVH |
| TYY1_MOUSE | 1498 | HVCAECGKAFVESSKLKRHQLVH |
| ZFX2_MOUSE | 1499 | HICVECGKGFRHPSELKKHMRIH |
| ZFX1_MOUSE | 1500 | HICVECGKGFRRPSELKKHMRIH |
| ZFA_MOUSE | 1501 | HICVECGKGFCHPSELKKHMRIH |
| ZFY2_MOUSE | 1502 | HICGECGKGFRHPSALKKHIRVH |
| ZFY1_MOUSE | 1503 | FICGECGKGFRHPSALKKHIRVH |
| Q61116_MOUSE | 1504 | --CHECGKGFRQSSALQTHQRVH |
| Q06054_MOUSE | 1505 | YQCRKCGKCFRTYSSLYRHRRTH |
| Q9Z117_MOUSE | 1506 | HQCEKCRKCFSTASSLTVHKRIH |
| Q61898_MOUSE | 1507 | HQCGKCGKCFNTSSSLTVHHRIH |
| Q60585_MOUSE | 1508 | YDCKECGKAFRLFSQLTQHQSIH |
| Q60585_MOUSE | 1509 | YKCMECEKTFRLLSQLTQHQSIH |
| Q60585_MOUSE | 1510 | YDCKECGKAFRLHSSLIQHQRIH |
| KR2_MOUSE | 1511 | YQCKECGKAFRKNSSLIQHERIH |
| KID1_MOUSE | 1512 | YLCNECGNTFKSSSSLRYHQRIH |
| KR2_MOUSE | 1513 | YGCDECGKTFRQSSSLLKHQRIH |
| ZF37_MOUSE | 1514 | YKCNECGKTFRHSSNLMQHLRSH |
| Q62514_MOUSE | 1515 | YKCNECGKTFRHSSNLMQHLRSH |
| KID1_MOUSE | 1516 | YKCNECGKTFRCNSSLSNHQRTH |
| ZF37_MOUSE | 1517 | YECKECGKSFRYNSSLTEHVRTH |
| Q62514_MOUSE | 1518 | YECKECGKSFRYNSSLTEHVRTH |
| Q9Z117_MOUSE | 1519 | YKCKECGKSFLELSHLKRHYRIH |
| O88631_MOUSE | 1520 | HKCKECGKSFFILSHLKTHYRIH |
| Q61898_MOUSE | 1521 | YECKECGKSFIELSHLKRHYRIH |
| Q9Z1D7_MOUSE | 1522 | HGCDECGKSFTQHSRLIEHKRVH |
| O35738_MOUSE | 1523 | FKCADCDRRFSRSDHLALHRRRH |
| O89090_MOUSE | 1524 | --CPECPKRFMRSDHLSKHIKTH |
| Q64167_MOUSE | 1525 | --CPECPKRFMRSDHLSKHIKTH |
| O89087_MOUSE | 1526 | --CPECPKRFMRSDHLSKHIKTH |
| Q62445_MOUSE | 1527 | --CPECSKRFMRSDHLSKHVKTH |
| O89091_MOUSE | 1528 | --CPMCDRRFMRSDHLTKHARRH |
| Q61596_MOUSE | 1529 | --CPMCDRRFMRSDHLTKHARRH |
| BTE1_MOUSE | 1530 | --CPLCEKRFMRSDHLTKHARRH |
| Q62445_MOUSE | 1531 | FICNWMFCGKRFTRSDELQRHRRTH |
| Q64167_MOUSE | 1532 | FMCNWSYCGKRFTRSDELQRHKRTH |
| O89090_MOUSE | 1533 | FMCNWSYCGKRFTRSDELQRHKRTH |
| O89087_MOUSE | 1534 | FMCNWSYCGKRFTRSDELQRHKRTH |
| Q60843_MOUSE | 1535 | YHCNWEGCGWKFARSDELTRHYRKH |
| EZF_MOUSE | 1536 | YHCDWDGCGWKFARSDELTRHYRKH |
| Q60980_MOUSE | 1537 | YKCTWEGCTWKFARSDELTRHFRKh |
| O35738_MOUSE | 1538 | YKCTWEGCTWKFGRSDELTRHYRKH |
| Q9Z0Z7_MOUSE | 1539 | YKCTWEGCDWRFARSDELTRHYRKH |
| O70261_MOUSE | 1540 | YACSWDGCDWRFARSDELTRHYRKH |
| EKLF_MOUSE | 1541 | YACSWDGCDWRFARSDELTRHYRKH |
| Q61596_MOUSE | 1542 | FSCSWKGCERRFARSDELSRHRRTH |
| O89091_MOUSE | 1543 | FSCSWKGCERRFARSDELSRHRRTH |
| BTE1_MOUSE | 1544 | FPCTWPDCLKKFSRSDELTRHYRTH |
| EGR2_MOUSE | 1545 | YPCPAEGCDRRFSRSDELTRHIRIH |
| WT1_MOUSE | 1546 | YQCDFKDCERRFSRSDQLKRHQRRH |
| WT1_MOUSE | 1547 | FQCKTCQRKFSRSDHLKTHTRTH |
| EGR1_MOUSE | 1548 | FQCRICMRNFSRSDHLTTHIRTH |
| KR2_MOUSE | 1549 | YQCNECGKPFSRSTNLTRHQRTH |
| O35700_MOUSE | 1550 | YTCRYCGKIFPRSANLTRHLRTH |
| EVI1_MOUSE | 1551 | YTCRYCGKIFPRSANLTRHLRTH |
| ZF29_MOUSE | 1552 | FQCAECGKSFSRSPNLIAHQRTH |
| ZF38_MOUSE | 1553 | YVCTKCGKAFSHSSNLTLHYRTH |
| Q9Z1D8_MOUSE | 1554 | YQCDSCGKAFSYSSDLIQHYRTH |
| ZF29_MOUSE | 1555 | YQCGECGKNFSRSSNLATHRRTH |
| ZF29_MOUSE | 1556 | YRCPECGKGFSNSSNFITHQRTH |
| ZF38_MOUSE | 1557 | YICAECGKAFSNSSNLIKHRRTH |
| ZF29_MOUSE | 1558 | YECLTCGESFSWSSNLIKHQRTH |
| ZF90_MOUSE | 1559 | YECNECGEAFSRLSSLTQHERTH |
| MLZ4_MOUSE | 1560 | YHCNECGENFSRISHLVQHQRTH |
| ZF29_MOUSE | 1561 | YKCLMCGKSFSRGSILVMHQRAH |
| MLZ4_MOUSE | 1562 | YECEECGKSFSRSSHLAQHQRTH |
| MLZ4_MOUSE | 1563 | YKCYECGKGFSRSSHLIQHQRTH |
| O70162_MOUSE | 1564 | FACPECGQRFSQRLKLTRHQRTH |
| O35483_MOUSE | 1565 | FPCPECGKRFSQRSVLVTHQRTH |
| O35483_MOUSE | 1566 | --CDECGKGFVYRSHLAIHQRTH |
| ZFP1_MOUSE | 1567 | YECSECGKSFIQNSQLIIHRRTH |
| GFI1_MOUSE | 1568 | HKCQVCGKAFSQSSNLITHSRKH |
| O70237_MOUSE | 1569 | HKCQVCGKAFSQSSNLITHSRKH |
| ZF29_MOUSE | 1570 | YKCTECGQKFSQSSALITHRRTH |
| KID1_MOUSE | 1571 | FKCKECSKAFSQSSALIQHQITH |
| KID1_MOUSE | 1572 | CKCKVCGKAFRQSSALIQHQRMH |
| Z151_MOUSE | 1573 | YVCERCGKRFVQSSQLANHIRHH |
| O35700_MOUSE | 1574 | YECENCAKVFTDPSNLQRHIRSQH |
| EVI1_MOUSE | 1575 | YECENCAKVFTDPSNLQRHIRSQH |
| Q60585_MOUSE | 1576 | YECKKCAKIFTCSSDLRGHQRSH |
| Q9Z116_MOUSE | 1577 | YECTVCRKSFICKSSFSHHWRTH |
| KR2_MOUSE | 1578 | YTCNVCDKHFIERSSLTVHQRTH |
| Q61164_MOUSE | 1579 | FQCSLCSYASRDTYKLKRHMRTH |
| P97365_MOUSE | 1580 | FQCWLCSAKFKISSDLKRHMRVH |
| KID1_MOUSE | 1581 | YKCSMCEKTFINTSSLRKHEKNH |
| ZF35_MOUSE | 1582 | YTCNLCSKSFSQSSDLTKHQRVH |
| ZF35_MOUSE | 1583 | YHCSSCNKAFRQSSDLILHHRVH |
| ZF38_MOUSE | 1584 | YWCSHCGKTFCSKSNLSKHQRVH |
| Q9Z1D9_MOUSE | 1585 | YKCGDCEKSFRQRSDLFKHQRTH |
| Q9Z1D9_MOUSE | 1586 | YKCDSCEKGFRQRSDLFKHQRIH |
| ZF35_MOUSE | 1587 | YPCSQCSKMFSRRSDLVKHYRIH |
| ZF35_MOUSE | 1588 | YQCSHCSKSFSQHSGMVKHLRIH |
| ZF35_MOUSE | 1589 | YACTQCPRSFSQKSDLIKHQRIH |
| ZF35_MOUSE | 1590 | YPCAQCNKSFSQNSDLIKHRRIH |
| ZF35_MOUSE | 1591 | YMCNHCYKHFSQSSDLIKHQRIH |
| ZF35_MOUSE | 1592 | YNCDECDQSFAWSTGLIRHQRTH |
| Q9Z1D9_MOUSE | 1593 | YQCQECGKRFSQSAALVKHQRTH |
| Q9Z1D9_MOUSE | 1594 | YACVVCGRRFSQSATLIKHQRTH |
| Q9Z116_MOUSE | 1595 | YECKQCMKTFYRKSGLTRHQRTH |
| Q06054_MOUSE | 1596 | YECKQCSKSFYTSSHLENHYRTH |
| Q9Z116_MOUSE | 1597 | YECQLCQKAFYCTSHLIVHQRTH |
| ZF29_MOUSE | 1598 | YECPQCGKTFSRKSHLITHERTH |
| MLZ4_MOUSE | 1599 | YECVQCGKGFTQSSNLITHQRVH |
| ZF37_MOUSE | 1600 | YECNHCGKVLSHKQGLLDHQRTH |
| Q62514_MOUSE | 1601 | YECNHCGKVLSHKQGLLDHQRTH |
| ZF90_MOUSE | 1602 | YECNECGRAFRKKTNLHDHQRTH |
| Q61491_MOUSE | 1603 | YECNQCGRAFRQYVYLQCHERIH |
| ZF35_MOUSE | 1604 | YPCAQCGKSFSQRSDLVNHQRVH |
| Q64247_MOUSE | 1605 | YVCEQCGKGFIQLKYLLMHQRSH |
| Q61116_MOUSE | 1606 | YTCQQCGKGFSQASYFHMHQRVH |
| O35483_MOUSE | 1607 | YRCVFCGAGFGRRSYCVTHQRTH |
| ZF29_MOUSE | 1608 | YRCGDCGKGFSQRSQLVVHQRTH |
| Q61117_MOUSE | 1609 | YRCDICGKRFRQRSYLHDHHRIH |
| Q9Z2U2_MOUSE | 1610 | FKCVVPSCTKTFTRNSNLRAHCQLVH |
| Q61116_MOUSE | 1611 | YRCDSCGKGFSRSSDLNIHRRVH |
| Q61117_MOUSE | 1612 | YQCHACWKSFCHSSEFNNHIRVH |
| Z239_MOUSE | 1613 | YQCYECGKGFSQSSDLRIHLRVH |
| Z239_MOUSE | 1614 | FKCDRCGKGFSQSSKLHIHKRVH |
| Z239_MOUSE | 1615 | YHCGKCGQGFSQSSKLLIHQRVH |
| Z239_MOUSE | 1616 | YKCGECGKGFSQSSNLHIHRCTH |
| ZF35_MOUSE | 1617 | YKCDECGKAFSQSSDLMIHQRIH |
| ZF38_MOUSE | 1618 | YDCKCGKAFGQSSDLLKHQRMH |
| O35700_MOUSE | 1619 | YRCKYCDRSFSISSNLQRHVRNIH |
| EVI1_MOUSE | 1620 | YRCKYCDRSFSISSNLQRHVRNIH |
| O35483_MOUSE | 1621 | YRCVFCGRSFSQSSALARHQAVH |
| O35483_MOUSE | 1622 | YLCSNCGRRFSQSSHLLTHMKTH |
| O70162_MOUSE | 1623 | FVCGECGRSFSRSSHLLRHQLTH |
| O88412_MOUSE | 1624 | YECAKCGAAFISNSHLMRHHRTH |
| O88631_MOUSE | 1625 | YKCMECDRSYIQYSHLKRHQKVH |
| O88631_MOUSE | 1626 | YKCKECGKSYAYRTGLKRHQKIH |
| Z239_MOUSE | 1627 | YECSKCGKGFSQSSNLHIHQRVH |
| Z239_MOUSE | 1628 | YACEECGMSFSQRSNLKIHQRVH |
| MLZ4_MOUSE | 1629 | YECNECWRSFGERSDLIKHQRTH |
| MLZ4_MOUSE | 1630 | YECHECGRGFSERSDLIKHYRVH |
| Q61116_MOUSE | 1631 | YECNECGKRFSLSGNLDIHQRVH |
| Q61116_MOUSE | 1632 | YKCGDCGKRFSCSSNLHTHQRVH |
| Q62518_MOUSE | 1633 | YKCGECGKSFICSSNLYIHQRVH |
| Q9Z150_MOUSE | 1634 | CPRCGKQFNHSSNLNRHMNVHRG |
| Q61116_MOUSE | 1635 | FHCSVCGKNFSRSSHFLDHQRIH |
| Q61116_MOUSE | 1636 | KCNVCQKQFSKTSNLQAHQRVH |
| Q62518_MOUSE | 1637 | YSCDVCGKGFSRSSQLQSHQRVH |
| Q62518_MOUSE | 1638 | FKCDACGKSFSRSSHLRSHQRVH |
| Q61898_MOUSE | 1639 | YKCRECDKSFTQRAYLRNHHNRVH |
| Q61898_MOUSE | 1640 | YKCMECDKSFTHNSNFRTHQRVH |
| Q9Z117_MOUSE | 1641 | YKCMECNKSFTQDSHLRTHQRVH |
| Q61898_MOUSE | 1642 | YKCIECDKSFTQVSHLRTHQRVH |
| O88631_MOUSE | 1643 | YKCSECDKSFTQASQLRTHQRVH |
| Q61898_MOUSE | 1644 | YKCNECDRSFTHYASLRWHQKTH |
| Q9Z117_MOUSE | 1645 | YKCKECDKSFAHCSSFRRHQKTH |
| Q61898_MOUSE | 1646 | YKCKECDKSFAHYPNFRTHQKIH |
| O88631_MOUSE | 1647 | YKCKDCDIFFNHYSSLRRHQKVH |
| Q9Z117_MOUSE | 1648 | YKCKDCDISFIQISNLRRHQRVH |
| Q61898_MOUSE | 1649 | YKCRDCDISFSQISNLRRHQKLH |
| Q9Z117_MOUSE | 1650 | FKCRECDKSFTKCSHLRRHQSVH |
| Q61898_MOUSE | 1651 | YKCRECDKSFIHSSHLRRHQNVH |
| Q9Z117_MOUSE | 1652 | YKCRECDKSFIQRSNLIIHQRVH |
| Q06054_MOUSE | 1653 | YKCSECEKSFTCGSVLRKHQKIH |
| Q06054_MOUSE | 1654 | YKCSECEKSFTVGSDLRMHQKIH |
| Q06054_MOUSE | 1655 | YKCSECEKCFTVVSDLRTHQKIH |
| Q06054_MOUSE | 1656 | YKCSECEKSFTVGSSLRTHQRIH |
| Q06054_MOUSE | 1657 | YKCECGKSPTVGSDLRKHQKCH |
| Q61898_MOUSE | 1658 | YKCIECGKSFTNNSYLRTHQKVH |
| Q61898_MOUSE | 1659 | YRCKECDKSFHESATLREHEKSH |
| Q61898_MOUSE | 1660 | YRCAECDKSFTRCSYLRAHQKIH |
| Q9Z117_MOUSE | 1661 | YRCKECDKSFTECSTLRAHQKIH |
| Q61898_MOUSE | 1662 | YRCKECDKSFTSCSTLKAHQSIH |
| Q9Z117_MOUSE | 1663 | YICKECGKSFTRCSYLRAHQKIH |
| O88631_MOUSE | 1664 | YVCKECGKSLTTCAILRAHQKIH |
| Q61898_MOUSE | 1665 | YECKECGKSFTTCSTLRIHQTIH |
| Q9Z117_MOUSE | 1666 | YICKECGKSFTKCSTLQIHQKIH |
| O88631_MOUSE | 1667 | YTCKQCGKSFTRGSTLRVHQRIH |
| O88631_MOUSE | 1668 | YKCNICDKSFTECSSLKEHRKIH |
| Q9Z117_MOUSE | 1669 | YKCEVCDKSFTVNSTLKTHLKIH |
| Q61898_MOUSE | 1670 | YKCEICDKSFTTTTTLKTHQKIH |
| Q9Z117_MOUSE | 1671 | YKCSVCGKSFTQCTNLKTHQRLH |
| Q61898_MOUSE | 1672 | YKCSVCDKSFTQCTRLKIHQRRH |
| KID1_MOUSE | 1673 | YRCKECGKSFGRRSGLFIHQKVH |
| ZF29_MOUSE | 1674 | YSCPECGKSFGNRSSLNTHQGIH |
| Q9Z117_MOUSE | 1675 | YKCKECGKSFPQLSALKSHQKIH |
| Q61898_MOUSE | 1676 | YKCKECEKSFVQLSALKSHQKLH |
| O88631_MOUSE | 1677 | YKCNDCGKSFSYLSALQSHHKRH |
| Q08376_MOUSE | 1678 | FVCEMCTKGFTTQAHLKEHLKIH |
| Q60636_MOUSE | 1679 | FKCQTCNKGFTQLAHLQKHYLVH |
| Q61116_MOUSE | 1680 | YKCEVCGKGFTQWAHLQAHERIH |
| O88282_MOUSE | 1681 | YKCETCGSRFVQVAHLRAHVLIH |
| Q61065_MOUSE | 1682 | YKCETCGARFVQVAHLRAHVLIH |
| BCL6_MOUSE | 1683 | YKCETCGARFVQVAHLRAHVLIH |
| O88631_MOUSE | 1684 | YRCEVCDKWFTLSSSLSRHQKIH |
| Q61116_MOUSE | 1685 | YRCEVCGKRFPWSLSLHSHQSVH |
| Z239_MOUSE | 1686 | YKCDKCGKGFTRSSSLLVHHSLH |
| ZF29_MOUSE | 1687 | YKCGLCGKSFSQSSSLIAHQGTH |
| Q62518_MOUSE | 1688 | YKCVDCGKEFSRPSSLQAHQGIH |
| Q61117_MOUSE | 1689 | YRCEECGKGFSWSSSLLIHQRAH |
| Q61117_MOUSE | 1690 | YKCEECGKVFSWSSYLKAHQRVH |
| Q61116_MOUSE | 1691 | FKCEECGKEFRWSVGLSSHQRVH |
| Q61117_MOUSE | 1692 | YKCETCGKAFSRVSILQVHQRVH |
| Q61116_MOUSE | 1693 | YKCEECGKGFSSASSFQSHQRVH |
| Q61116_MOUSE | 1694 | YKCGECGKGFSHASSLQAHHSVH |
| Q61117_MOUSE | 1695 | YQCAECGRGFTVESHLQAHQRSH |
| Q61117_MOUSE | 1696 | YQCEECGRGFCRASNFLAHRGVH |
| Q61117_MOUSE | 1697 | YKCEECGKGFTRASTLLDHQRGH |
| Q61117_MOUSE | 1698 | YVCEECGKGFSQASHLLAHQRGH |
| Q62518_MOUSE | 1699 | YNCETCGSAFSQASHLQDHQRLH |
| ZF29_MOUSE | 1700 | YRCPECGKGFSWNSVLIIHQRIH |
| O70162_MOUSE | 1701 | YCCGECDLGFTQVSRLTEHQRIH |
| KID1_MOUSE | 1702 | YRCSECGKGFTSISRLNRHRIIH |
| TYY1_MOUSE | 1703 | YVCPFDGCNKKFAQSTNLKSHILTH |
| REX1_MOUSE | 1704 | YQCTFEGCGKRFSLDFNLRTHIRIH |
| TYY1_MOUSE | 1705 | FQCTFEGCGKRFSLDFNLRTHVRIH |
| MTF1_MOUSE | 1706 | YQCTFEGCPRTYSTAGNLRTHQKTH |
| GLI_MOUSE | 1707 | HKCTFEGCRKSYSRLENLKTHLRSH |
| GLI3_MOUSE | 1708 | NKCTFEGCTKAYSRLENLKTHLRSH |
| ZIC2_MOUSE | 1709 | FQCEFEGCDRRFANSSDRKKHMHVH |
| ZIC1_MOUSE | 1710 | FKCEFEGCDRRFANSSDRKKHMHVH |
| ZIC3_MOUSE | 1711 | PKCEFEGCDRRFANSSDRKKHMHVH |
| ZIC4_MOUSE | 1712 | FRCEFEGCERRFANSSDRKKHSHVH |
| GLI_MOUSE | 1713 | YMCEQEGCSKAFSNASDRAKHQNRTH |
| GLI3_MOUSE | 1714 | YVCEHEGCNKAFSNASDRAKHQNRTH |
| O70230_MOUSE | 1715 | YVCTVPGCDKRFTEYSSLYKHHVVH |
| MTF1_MOUSE | 1716 | FECDVQGCEKAFNTLYRLKARQRLH |
| MTF1_MOUSE | 1717 | FVCNQEGCGKAFLTSYSLRIHVRVH |
| O70230_MOUSE | 1718 | YQCEHSGCGKAFATGYGLKSHFRTH |
| MTF1_MOUSE | 1719 | FRCDHDGCGKAFAASHHLKTHVRTH |
| O70230_MOUSE | 1720 | FKCPIEGCGRSFTTSNIRKVHIRTH |
| ZIC4_MOUSE | 1721 | FPCPFPGCGKVFARSENLKIHKRTH |
| ZIC2_MOUSE | 1722 | FPCPFPGCGKVFARSENLKIHKRTH |
| ZIC1_MOUSE | 1723 | FPCPFPGCGKVFARSENLKTHKRTH |
| ZIC3_MOUSE | 1724 | FPCPFPGCGKIFARSENLKIHKRTH |
| O70230_MOUSE | 1725 | YYCTEPGCGRAFASATNYKNHVRIH |
| O70230_MOUSE | 1726 | YRCSEDNCTKSFKTSGDLQKHIRTH |
| MTF1_MOUSE | 1727 | FNCESQGCSKYFTTLSDLRKHIRTH |
| O70230_MOUSE | 1728 | FRCKYDGCGKLYTTAHHLKVHERSH |
| BTE1_MOUSE | 1729 | HKCPYSGCGKVYGKSSHLKAHYRVH |
| Q9Z0Z7_MOUSE | 1730 | --CDYNGCTKVYTKSSHLKAHLRTH |
| Q60980_MOUSE | 1731 | HRCDYDGCNKVYTKSSHLKAHRRTH |
| O35738_MOUSE | 1732 | HRCDFEGCNKVYTKSSHLKAHRRTH |
| Q61596_MOUSE | 1733 | HICSHPGVGKTYFKSSHLKAHVRTH |
| O89091_MOUSE | 1734 | HICSHPGCGKTYFKSSHLKAHVRTH |
| Q60843_MOUSE | 1735 | HTCSYTNCGKTYTKSSHLKAHLRTH |
| EZF_MOUSE | 1736 | HTCDYAGCGKTYTKSSHLKAHLRTH |
| Q64167_MOUSE | 1737 | HICHIQGCGKVYGKTSHLRAHLRWH |
| O89090_MOUSE | 1738 | HICHIQGCGKVYGKTSHLRAHLRWH |
| O89087_MOUSE | 1739 | HICHIQGCGKVYGKTSHLRAHLRWH |
| Q62445_MOUSE | 1740 | HVCHIEGCGKVYGKTSHLRAHLRWH |
| O70261_MOUSE | 1741 | HTCGHEGCGKSYTKSSHLKAHLRTH |
| EKLF_MOUSE | 1742 | HTCGHEGCGKSYSKSSHLKAHLRTH |
| WT1_MOUSE | 1743 | FMCAYPGCNKRYFKLSHLQMHSRKH |
| ZEP1_MOUSE | 1744 | YICEYCNRACAKPSVLLKHIRSH |
| Q61479_MOUSE | 1745 | YICQYCSRPCAKPSVLQKHIRSH |
| O55140_MOUSE | 1746 | YICPYCSRACAKPSVLKKHIRSH |
| Q60636_MOUSE | 1747 | HECQVCHKRFSSTSNLKTHLRLH |
| SNAI_MOUSE | 1748 | CVCTTCGKAFSRPWLLQGHVRTH |
| P97469_MOUSE | 1749 | CVCKICGKAFSRPWLLQGHIRTH |
| ZIC2_MOUSE | 1750 | HVCFWEECPREGKPFKAKYKLVNHIRVH |
| ZIC3_MOUSE | 1751 | HVCYWEECPREGKSFKAKYKLVNHIRVH |
| Q62065_MOUSE | 1752 | HECKLCGASFRTKGSLIRHHRRH |
| Q62065_MOUSE | 1753 | HVCQFCSRGFREKGSLVRHVRHH |
| IKAR_MOUSE | 1754 | FQCNQCGASFTQKGNLLPHIKLH |
| Q9Z2Z2_MOUSE | 1755 | FHCNQCGASFTQKGNLLRHIKLH |
| HELI_MOUSE | 1756 | FHCNQCGASFTQKGNLLRHIKLH |
| Q61164_MOUSE | 1757 | HKCHLCGRAFRTVTLLRNHLNTH |
| Q61624_MOUSE | 1758 | HVCEHCNAAFRTNYHLQRHVFIH |
| P97475_MOUSE | 1759 | HVCEHCNAAFRTNYHLQRHVFIH |
| Z151_MOUSE | 1760 | YVCTHCQRQFADPGGLQRHVRIH |
| Q62511_MOUSE | 1761 | YICEYCARAFKSSHNIAVHRMIH |
| MAZ_MOUSE | 1762 | YICALCAKEFKNGYNLRRHEAIH |
| O88939_MOUSE | 1763 | YECNICKVRFTRQDKLKVHMRKH |
| Q64321_MOUSE | 1764 | --CEVCGVRFTRNDKLKIHMRKH |
| P97365_MOUSE | 1765 | PHKCEVCGKCFSRKDKLKTHMRCH |
| O88939_MOUSE | 1766 | YLCQQCGAAFAHNYDLKNHMRVH |
| Q64321_MOUSE | 1767 | YSCPHCPARFLHSYDLKNHMHLH |
| Z151_MOUSE | 1768 | HKCEDCGKEFTHTGNFKRHIRIH |
| Z151_MOUSE | 1769 | YRCGDCGKLFTTSGNLKRHQLVH |
| Z151_MOUSE | 1770 | -KCRECGKQFTTSGNLKRHLRIH |

Chicken database.

| 35 | SEQ | |
|---|---|---|
| finger units | ID NO | |
| Q92010_CHICK | 1771 | YSCEVCGKSFIRAPDLKKHERVH |
| Q90851_CHICK | 1772 | YPCTICGKKFTQRGTMTRHMRSH |
| Q90850_CHICK | 1773 | YPCTICGKKFTQRGTMTRHMRSH |
| Q90851_CHICK | 1774 | --CDACGMRFTRQYRLTEHMRIH |
| Q90850_CHICK | 1775 | --CDACGMRFTRQYRLTEHMRIH |
| CTCF_CHICK | 1776 | HKCPDCDMAFVTSGELVRHRRYKH |
| ZKR1_CHICK | 1777 | -TCGDCGKGFAWASHLQRHRRVH |
| ZKR1_CHICK | 1778 | HRCGDCGKGFAWASHLQRHRRVH |
| ZKR1_CHICK | 1779 | HRCGDCGKGFVWASHLERHRRVH |
| ZKR1_CHICK | 1780 | --CPDCGKSFPWASHLERHRRVH |
| Q92010_CHICK | 1781 | --CHMCDKAFKHKSHLKDHERRH |
| O42408_CHICK | 1782 | HECGICKKAFKHKHHLIEHMRLH |
| DEFI_CHICK | 1783 | HECGICKKAFKHKHHLIEHMRLH |
| O42408_CHICK | 1784 | FKCTECGKAFKYKHHLKEHLRIH |
| DEFI_CHICK | 1785 | FKCTECGKAFKYKHHLKEHLRIH |
| O42409_CHICK | 1786 | YPCQYCGKRFHQKSDMKKHTYIH |
| O42409_CHICK | 1787 | FECKMCGKTFKRSSTLSTHLLIH |
| ZKR1_CHICK | 1788 | YECPECGEAFSQGSHLTKHRRSH |
| ZKR1_CHICK | 1789 | YECPECGEAFSQGSHLTKHRRSH |
| ZKR1_CHICK | 1790 | YSCPECGESYSQSSHLVQHRRTH |
| O42409_CHICK | 1791 | HKCQVCGKAFSQSSNLITHSRKH |
| O57415_CHICK | 1792 | YQCNICDYIAADKAALIRHLRTH |
| CTCF_CHICK | 1793 | FQCSLCSYASRDTYKLKRHMRTH |
| O57415_CHICK | 1794 | YKCQTCERTFTLKHSLVRHQRIH |
| Q92010_CHICK | 1795 | FVCEMCTKGFTTQAHLKEHLKIH |
| O57415_CHICK | 1796 | -TCPYCPRVFSWASSLQRHMLTH |
| O57415_CHICK | 1797 | HSCSICGKSLSSASSLDRHMLVH |
| O57415_CHICK | 1798 | --CTVCNKRFWSLQDLTRHMRSH |
| Q91051_CHICK | 1799 | CVCKICGKAFSRPWLLQGHIRTH |
| O12939_CHICK | 1800 | CVCKMCGKAFSRPWLLQGHIRTH |
| O57415_CHICK | 1801 | YKCSVCGQSFTTNGNMHRRMKIH |
| IKAR_CHICK | 1802 | FQCNQCGASFTQKGNLLRHIKLH |
| CTCF_CHICK | 1803 | HKCHLCGRAFRTVTLLRNHLNTH |
| O93567_CHICK | 1804 | YECNICNVRFTRQDKLKVHMRKH |
| O93567_CHICK | 1805 | YLCQQCGAAFARNYDLKNHMRVH |

Plant Database.

| 52 | SEQ | |
|---|---|---|
| finger units | ID NO | |
| O22089_PETHY | 1806 | HECSICGEQFLLGQALGGHMRKH |
| O22088_PETHY | 1807 | HECSFCGEDFPTGQALGGHMRKH |
| O22087_PETHY | 1808 | -ECSPCGEDFPTGQALGGHMRKH |
| Q39092_ARATH | 1809 | HKCKLCWKSFANGRALGGHMRSH |
| Q39217_ARATH | 1810 | HKCSICSQSFGTGQALGGHMRRH |
| P93713_PETHY | 1811 | HECSICGLEFAIGQALGGHMRRH |
| O22086_PETHY | 1812 | HECSICGLEFPIGQALGGHMRRH |
| O22085_PETHY | 1813 | HECSICGMEFSLGQALGGHMRRH |
| O22084_PETHY | 1814 | HECSICGMEFSLGQALGGHMRRR |
| Q42453_ARATH | 1815 | HPCPICGVKFPMGQALGGHMRRH |
| Q42410_ARATH | 1816 | HPCPICGVEFPMGQALGGHMRRH |
| O65150_TOBAC | 1817 | HVCSICHKAFPTGQALGGHKRRH |
| Q40897_PETHY | 1818 | HVCSICHKAFPTGQALGGHKRRH |
| Q40896_PETHY | 1819 | HVCSICHKAFPSGQALGGHKRRH |
| Q42430_WHEAT | 1820 | HRCSICQKEFPTGQALGGHKRKH |
| Q40899_PETHY | 1821 | HECSICHKCFPTGQALGGHKRCH |
| P93166_SOYBN | 1822 | HECSICHKSFPTGQALGGHKRCH |
| Q96289_ARATH | 1823 | HVCTICNKSFPSGQALGGHKRCH |
| Q42423_ARATH | 1824 | HVCTICNKSFPSGQALGGHKRCH |
| O22533_ARATH | 1825 | HVCSICHKSFATGQALGGHKRCH |
| Q40898_PETHY | 1826 | HECSICHKCFSSGQALGGHKRRH |
| Q38895_ARATH | 1827 | YTCSFCKREFRSAQALGGHMNVH |
| O23621_APATH | 1828 | YTCNFCRREFRSAQALGGHMNVH |
| O80942_APATH | 1829 | YTCSFCRREFKSAQALGGHMNVH |
| P93714_PETHY | 1830 | HECSYCGAEFTSGQALGGHMRRH |
| Q43614_PETHY | 1831 | HECAICGAEFTSGQALGGHMRRH |
| O22083_PETHY | 1832 | HECSICGAEFTSGQALGGHMRRH |
| Q41070_PEA | 1833 | HECSICGAEFTSGQALGGHMRRH |
| Q42375_APATH | 1834 | HECSICGSEFTSGQALGGHMRRH |
| O65499_ARATH | 1835 | HKCNICFRVFSSGQALGGHMRCH |
| O22090_PETHY | 1836 | HECPVCFRVFSSGQALGGHKRTH |
| O22082_PETHY | 1837 | HECPVCYRVFSSGQALGGHKRSH |
| P93717_PETHY | 1838 | HECSICHRVFSTGQALGGHKRCH |
| O04177_BRARA | 1839 | HTCSICFKSFSSGQALGGHKRCH |
| O04176_BRARA | 1840 | HTCSICFKSFSSGQALGGHKRCH |
| P93715_PETHY | 1841 | HQCSICHRVFSSGQALGGHKRCH |
| Q39092_APATH | 1842 | HECPICAKVFTSGQALGGHKRSH |
| P93719_PETHY | 1843 | HECPYCDRVFKSGQALGGHKRSH |
| P93718_PETHY | 1844 | HACPFCPRMFKSGQALGGHKRSH |
| O22091_PETHY | 1845 | YECPLCFKIFQSGQALGGHKRSH |
| Q42430_WHEAT | 1846 | -KCSVCGKSFSSYQALGGHKTSH |
| O04177_BRARA | 1847 | YKCTVCGKSFSSYQALGGHKTSH |
| O04176_BRARA | 1848 | YKCTVCGKSFSSYQALGGHKTSH |
| Q96289_ARATH | 1849 | YKCSVCDKTFSSYQALGGHKASH |
| Q42423_ARATH | 1850 | YKCSVCDKTFSSYQALGGHKASH |
| Q40897_PETHY | 1851 | YKCSVCDKSFSSYQALGGHKASH |
| Q40896_PETHY | 1852 | YKCSVCDKSFSSYQALGGHKASH |
| Q40898_PETHY | 1853 | YKCNVCNKSFHSYQALGGHKASH |
| O65150_TOBAC | 1854 | YKCSVCDKAFSSYQALGGHKASH |
| P93166_SOYBN | 1855 | YKCSVCDKSFPSYQALGGHKASH |
| Q40899_PETHY | 1856 | YKCSVCGKGFGSYQALGGHKASH |
| O22533_APATH | 1857 | YKCSVCDKAFSSYQALGGHKASH |

Arabidopsis Database

| | SEQ | |
|---|---|---|
| | ID NO | |
| Q9ZU64/169-191 | 1858 | YTCPKCNSIFDTSQKFAAHMSSH |
| O23621/40-62 | 1859 | YTCNFCRREFRSAQALGGHMNVH |
| O23504/5-27 | 1860 | HKCKLCSKSFCNGRALGGHMKSH |
| Q9SYC5/250-275 | 1861 | WYCSCGSDFKHKRSLKDHVKAFGNGH |
| Q9SYC5/224-246 | 1862 | FACRMCGKAFAVKGDWRTHEKNC |
| O22533/89-111 | 1863 | YKCSVCDKAFSSYQALGGHKASH |
| O22533/148-170 | 1864 | HVCSICHKSFATGQALGGHKRCH |
| Q9SN24/149-171 | 1865 | HNCSICFKSFPSGQALGGHKRCH |
| Q9SN24/94-116 | 1866 | YKCSVCGKSFPSYQALGGHKTSH |
| Q9STI7/117-140 | 1867 | YFCGVCDRRFYTNEKLINHFKQIH |
| Q9STM3/ | 1868 | LKCPWKGCKMTFKWAWSRTEHIRVH |
| 1296-1320 | | |
| Q9STM3/ | 1869 | YQCNMEGCTMSFSSEKQLMLHKRNIC |
| 1243-1268 | | |
| Q9STM3/ | 1870 | KGCGKNFFSHKYLVQHQRVH |
| 1271-1290 | | |
| Q9STM3/ | 1871 | YVCAEPDCGQTFRFVSDFSRHKRKTGH |
| 1326-1352 | | |
| Q9STM3/ | 1872 | LKCPWKGCKMTFKWAWSRTEHIRVH |
| 1296-1320 | | |
| O81801/142-164 | 1873 | PMCNVCGKGFASWKAVFGHLRQH |
| O65601/61-83 | 1874 | QKCEKCSREFCSPVNFRRHNRMH |
| Q9SFY6/118-140 | 1875 | YKCSVCDKTFSSYQALGGHKASH |
| Q9SFY6/174-196 | 1876 | HVCTICNKSFPSGQALGGHKRCH |
| O65245/147-171 | 1877 | FYCELCSKQYRTVMEFEGHLSSYDH |
| Q39261/52-74 | 1878 | FSCNYCQRKFYSSQALGGHQNAH |
| Q9SSW0/118-140 | 1879 | HVCSVCGKSFATGQALGGHKRCH |
| Q9SSW0/75-97 | 1880 | YKCGVCYKTFSSYQALGGHKASH |
| Q39262/61-83 | 1881 | FSCNYCQRTFYSSQALGGHQNAH |
| Q9SSW1/164-186 | 1882 | HTCSICFKSFASGQALGGHKRCH |
| Q9SSW1/97-119 | 1883 | YKCTVCGKSFSSYQALGGHKTSH |
| Q9ZPT0/145-167 | 1884 | WVCERCSKGYAVQSDYKAHLKTC |
| Q9ZPT0/67-89 | 1885 | YICEICNQGFQRDQNLQMHRRRH |
| Q9ZPT0/172-193 | 1886 | HSCDCGRVFSRVESFIEHQDNC |
| Q39263/85-107 | 1887 | FSCNYCQRKFYSSQALGGHQNAH |
| Q9SGD1/291-316 | 1888 | WYCTCGSDFKHKRSLKDHIRSFGSGH |
| Q9SGD1/265-287 | 1889 | FSCGKCGKALAVKGDWRTHEKNC |
| Q9SGD1/180-202 | 1890 | FACSICSKTFNRYNNMQMHMWGH |
| Q9SSW2/106-128 | 1891 | YKCNVCEKAFPSYQALGGHKASH |
| Q9SSW2/165-187 | 1892 | HECSICHKVFPTGQALGGHKRCH |
| Q39264/60-82 | 1893 | HECQYCGKEFANSQALGGHQNAH |
| P93815/7-30 | 1894 | QECAVCKRVFLSSHQLISHYNAAH |
| Q39265/41-63 | 1895 | YECQYCCREFANSQALGGHQNAH |
| Q39266/59-81 | 1896 | FSCNYCRRKFYSSQALGGHQNAH |
| Q39267/93-115 | 1897 | FECHYCFRNFPTSQALGGHQNAH |
| Q9SVY1/301-323 | 1898 | FMCRKCGKAFAVRGDWRTHEKNC |
| Q9SVY1/217-239 | 1899 | FSCPVCFKTFNRYNNMQMHMWGH |
| Q9SGH2/ | 1900 | IHCLICHKTFASDDEFEDHTESKC |
| 1804-1827 | | |
| Q38895/47-69 | 1901 | YTCSFCKREFRSAQALGGHMNVH |
| Q9SLB8/49-71 | 1902 | YTCSFCRREFRSAQALGGHMNVH |
| Q9SL35/188-210 | 1903 | HECSICGSEFTSGQALGGHMRRH |
| Q9SL35/113-135 | 1904 | YECKTCNRTFSSFQALGGHRASH |
| O81013/49-71 | 1905 | HFCVICEKQFSSGKAYGGHVRIH |
| O81013/119-141 | 1906 | IRCCLCGKEFQTMHSLFGHMRRH |
| O23395/664-686 | 1907 | LHCEKCGKALQPTEMEKHLKVFH |
| Q9SI97/34-56 | 1908 | FACKTCNKEFPSFQALGGHRASH |
| Q9SI97/78-100 | 1909 | HECPICGAEFAVGQALGGHMRKH |
| Q9SR34/35-57 | 1910 | YVCSFCIRGFSNAQALGGHMNIH |
| Q42485/68-90 | 1911 | FSCNYCQRKFYSSQALGGHQNAH |
| O82389/126-149 | 1912 | FPCNSCGEIFPKINLLENHIAIKH |
| Q9SQX8/182-204 | 1913 | YQCKTCDRTFPSFQALGGHRASH |
| Q9SQX8/261-283 | 1914 | HECGICGAEFTSGQALGGHMRRH |
| O65499/222-244 | 1915 | HKCNICFRVFSSGQALGGHMRCH |
| O65499/77-99 | 1916 | RPCTECGRKFWSWKALFGHMRCH |
| O65499/162-184 | 1917 | FECGGCKKVFGSHQALGGHRASH |
| Q9SCM4/220-243 | 1918 | DVCPKCSRGFRDPVDLLKHIDKDH |
| Q96289/80-102 | 1919 | YKCSVCDKTFSSYQALGGHKASH |
| Q96289/136-158 | 1920 | HVCTICNKSFPSGQALGGHKRCH |
| Q9SCQ6/139-161 | 1921 | WKCDKCSKKYAVQSDWKAHSKIC |
| Q9SCQ6/166-187 | 1922 | YKCDCGTLFSRRDSFITHRAFC |
| Q9SCQ6/63-85 | 1923 | FVCEICNKGFQRDQNLQLHRRGH |
| Q9SFS1/70-92 | 1924 | YVCEICNQGFQRDQNLQMHRRRH |
| Q9SFS1/148-170 | 1925 | WICERCSKGYAVQSDYKAHLKTC |
| Q9SFS1/175-196 | 1926 | HSCDCGRVFSRVESFIEHQDTC |
| Q9SSA6/575-598 | 1927 | IHCLICHKTFASDDEFEDHTESKC |
| Q42410/39-61 | 1928 | FTCKTCLKQFHSFQALGGHRASH |
| Q42410/82-104 | 1929 | HPCPICGVEFPMGQALGGHMRRH |
| Q9XFP6/12-35 | 1930 | VWCYYCDREFDDEKILVQHQKAKH |
| Q9XFP6/36-59 | 1931 | FKCHVCHKKLSTASGMVIHVLQVH |
| O22238/218-241 | 1932 | VSCGSCKKTFNSGNALESHNKAKH |
| Q42453/40-62 | 1933 | FRCKTCLKEFSSFQALGGHRASH |
| Q42453/86-108 | 1934 | HPCPICGVKFPMGQALGGHMRRH |
| Q42375/113-135 | 1935 | YECKTCNRTFSSFQALGGHRASH |
| Q42375/188-210 | 1936 | HECSICGSEFTSGQALGGHMRRH |
| O22759/159-181 | 1937 | WKCEKCSKFYAVQSDWKAHTKIC |
| O22759/186-207 | 1938 | YRCDCGTLFSRKDTFITHRAFC |
| O22759/82-104 | 1939 | FVCEICNKGFQRDQNLQLHRRGH |
| Q9ZUL3/81-103 | 1940 | FICEVCNKGFQREQNLQLHRRGH |
| Q9ZUL3/157-179 | 1941 | WKCDKCSKRYAVQSDWKAHSKTC |
| Q9ZUL3/184-205 | 1942 | YRCDCGTLFSRRDSFITHRAFC |
| P93751/95-117 | 1943 | FECHYCFRNFPTSQALGGHQNAH |
| O81827/196-219 | 1944 | VSCHKCGEKFSKLEAAEAHHLTKH |
| Q9ZUL4/82-104 | 1945 | WKCEKCSKRYAVQSDWKAHSKTC |
| Q9ZUL4/109-130 | 1946 | YRCDCGTIFSRRDSYITHRAFC |
| Q9ZUL4/6-28 | 1947 | FICDVCNKGFQREQNLQLHRRGH |
| Q9SHD0/194-216 | 1948 | FKCETCGKVFKSYQALGGHRASH |
| Q9SHD0/243-265 | 1949 | HECPICFRVFTSGQALGGHKRSH |
| Q9SHD0/4-26 | 1950 | YKCRFCFKSFINGRALGGHMRSH |
| O64936/131-153 | 1951 | YQCNVCGRELPSYQALGGHKASH |
| O64936/179-201 | 1952 | HKCSICHREFSTGHSLGGHKRLH |
| Q9SIJ0/65-87 | 1953 | RPCTECGKQFGSLKALFGHMRCH |
| Q9SIJ0/148-170 | 1954 | FECDGCKKVFGSHQALGGHRATH |
| Q9SIJ0/211-233 | 1955 | HRCNICSRVFSSGQALGGHMRCH |
| Q9SLD4/47-69 | 1956 | FECKTCNKRFSSFQALGGHRASH |
| Q9SLD4/94-116 | 1957 | HKCSICSQSFGTGQALGGHMRRH |
| Q9ZU93/121-143 | 1958 | FECPICKNPFTSEEEVSVHVESC |
| Q9SFT3/177-200 | 1959 | CACPQCGEVFPKLESLEHHQAVRH |
| Q9ZQEO/244-266 | 1960 | YTCPKCNGVFNTSQKFAAHMSSH |
| Q42423/80-102 | 1961 | YKCSVCDKTFSSYQALGGHKASH |
| Q42423/136-158 | 1962 | HVCTICNKSFPSGQALGGHKRCH |
| Q9ZWA6/146-168 | 1963 | WKCEKCAKRYAVQSDWKAHSKTC |
| Q9ZWA6/173-194 | 1964 | YRCDCGTIFSRRDSFITHRAFC |
| Q9ZWA6/70-92 | 1965 | FLCEICGKGFQRDQNLQLHRRGH |
| O80942/39-61 | 1966 | YTCSFCRREFKSAQALGGHMNVH |
| Q39217/90-112 | 1967 | HKCSICSQSFGTGQALGGHMRRH |
| Q39217/43-65 | 1968 | FECKTCNKRFSSFQALGGHRASH |
| Q39092/160-182 | 1969 | FECETCEKVFKSYQALGGHRASH |
| Q39092/209-231 | 1970 | HECPICAKVFTSGQALGGHKRSH |
| Q39092/5-27 | 1971 | HKCKLCWKSFANGRALGGHMRSH |
| O81793/138-160 | 1972 | PVCHICGRGFGSWKAVFGHMRAH |
| O64828/530-553 | 1973 | LQCIPCGSHFGDKEQLLVHVQAVH |
| O64828/599-622 | 1974 | FVCKFCGLKFNLLPDLGRHHQAEH |
| O64828/496-519 | 1975 | FACAICLDSFVRRKLLEIHVEERH |
| O49591/251-278 | 1976 | FMCLYCNELCRPFSSLEAVRKHMEAKSH |
| O49591/26-50 | 1977 | LTCNACNMEFKDEEERNLHYKSDWH |
| O49591/90-114 | 1978 | YTCAICAKGYRSSKAREQHLQSRSH |

There follow several examples of how to construct and select DNA-binding sub-domains from libraries of natural zinc fingers.

### Example 4

### Human Zinc Finger Module ‘Mini-Library’

As a preliminary test of the efficacy of using natural zinc finger modules for constructing novel DNA-binding domains, a ‘mini-library’ of natural, human zinc finger modules is 10 generated. The mini-library comprises 8 zinc finger modules, which have the following nomenclature assigned to them in the human genome database: Zif268 finger 1, Zif268 finger 2, Sp1 finger 3, WT1 finger 1, O15391, O75626, ZN45 and Z165. Since there is more than one zinc finger module belonging to the zinc fingers proteins ZN45 and Z165, we have called the selected modules ZN45-(AAA) and Z165-(GCC) respectively, according to their predicted binding site. We have also predicted the binding sites for the zinc fingers O15391 and O75626. The preferred binding sites for Zif268 finger 1, Zif268 finger 2, Sp1 finger 3 and WT1 finger 1 are already known. The amino acid sequences of each of the stated modules, and their predicted or previously determined binding sequences are shown in Table 3.

Two 3-zinc finger peptide libraries are prepared, containing the 8 zinc finger modules stated. All novel 3-finger peptides contain a leader sequence, MAEERP (SEQ ID NO:16), at the start of the peptide and are tagged by the sequence LRQKDGGGSYPYDVPDYA (SEQ ID NO:1989) at the C-terminus. This sequence provides: (in the absence of a further C-terminal finger) a suitable terminus to the final α-helix of the peptide -LRQKD- (SEQ ID NO:1987) as found in wild-type Zif268; a short, flexible linker sequence, GGGS (SEQ ID NO:2121); and an HA-tag (YPYDVPDYA (SEQ ID NO:2122)), which is recognised by the HA-antibody. Adjacent zinc finger modules are fused using the linker peptide sequence TGEKP (SEQ ID NO:3). The peptide sequences described above are also displayed in Table 3.

In the first library (library 1), the 8 zinc finger modules are recombined in random order to create 3-finger peptides with all possible combinations of the 8 zinc finger modules. Such a procedure results in a library diversity of 512 (=8³), comprising peptides that are predicted to bind to any possible combination of the binding sites assigned in Table 3. Library 1 allows novel 3-finger domains to be selected as a unit, for specified 9 bp target sequences. Such 3-finger units may be used for the construction of poly-zinc finger peptides as described in Moore, M., Choo, Y. & Klug, A. (2001) Proc. Natl. Acad. Sci. USA 98:1432-1436; and WO 01/53480.

In the second library (library 2), the 8 zinc finger modules are randomly recombined to create 2-finger peptides which are all joined to the C-terminus of Zif268 finger 1. The invariant finger 1 acts as an anchor for the selection, both by providing extra affinity to stabilise the selection, and by fixing the register of the protein DNA interaction (as discussed supra). Such a library has a diversity of 64 (=8²), and allows novel 2-finger units to be selected for a given 6 bp target sequence. The resulting 2 finger units can be recovered by PCR and used in the construction of poly-zinc finger peptides (based on strings of 2-finger units), as described in WO 01/53480.

These two libraries (encoding 3-finger peptides) are screened, as described below, for the ability of their encoded proteins to bind three different 9 bp binding sequences: 5′-GCG-TGG-GCG-3′; 5′-GGA-TAA-GCG-3′; and 5′-GCC-GAG-TGG-3′.

As positive controls, the genes encoding the 3-finger peptides predicted to bind the above target sequences are specifically constructed and tested in a similar manner.

TABLE 3

Nomenclature, amino acid sequences and
known or predicted binding sequences
(“SITE”) of zinc finger modules and other
peptide units used in library construction.

| | FINGER/ | SEQ | | |
|---|---|---|---|---|
| x | UNIT | ID NO: | PEPTIDE SEQUENCE | SITE |
| 1 | ZIF268 F1 | 1979 | YACPVESCDRRFSRSDELTRHIRIH | GCG |
| 2 | ZIF268 F2 | 1980 | FQCRICMRNFSRSDHLSTHIRTH | TGG |
| 3 | Sp1 F3 | 1981 | FSCPICEKRFMRSDHLTKHARRH | GGG |
| 4 | WT1 F1 | 1982 | FMCAYPGCNKRYFKLSHLQMHSRKH | GAG |
| 5 | O15391 | 1983 | FVCPFDVCNRKFAQSTNLKTHILTH | TAA¹ |
| 6 | O75626 | 1984 | FKCQTCNKGFTQLAHLQKHYLVH | GGA¹ |
| 7 | ZN45-AAA | 1985 | YKCEECGKGFSQASNLLAHQRGH | AAA¹ |
| 8 | Z165-GCC | 1986 | YECNECGKSFAESSDLTRHRRIH | GCC¹ |
| 9 | leader | 16 | MAEERP | — |
| 10 | linker | 3 | TGEKP | — |
| 11 | G₃S-HA- | 1989 | LRQKDGGGSYPYDVPDYA* | — |
| | tag | | | |

¹Predicted binding site.
*indicates a translation stop codon.

a. Human Zinc Finger Mini-Library Construction.

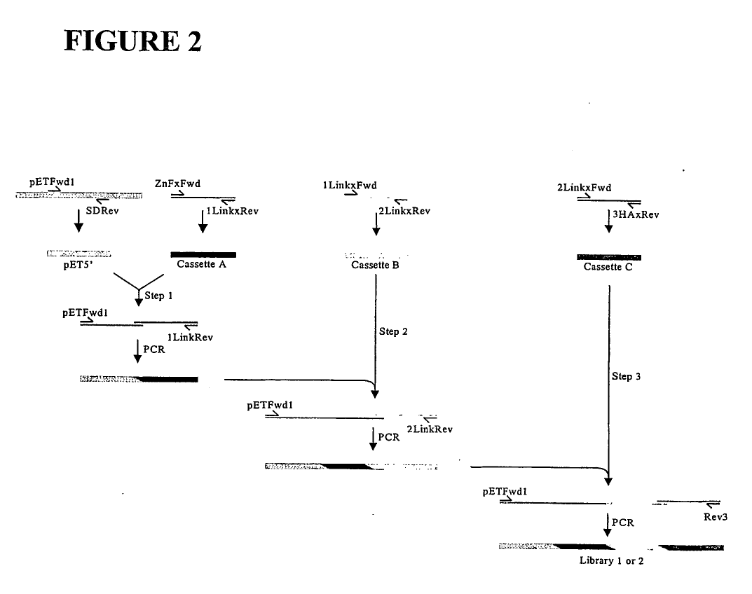

Two libraries are prepared, according to the scheme shown in FIG. 2. The N-terminal finger of the 3-finger construct is referred to as ‘cassette A’. The central finger is encoded by cassette B, and the third (C-terminal) finger module is called cassette C.

Zinc Finger Cassettes

Polynucleotide sequences encoding the amino acid sequences of the 8 zinc finger modules shown in Table 3 are determined, taking into account E. coli codon preferences, and the corresponding nucleotide sequences are synthesised as single stranded oligonucleotides, examples of which are shown in Table 4. Also shown are the sequences of exemplary linkers and an exemplary 3′-tag required for the assembly of 3-finger domains. Double stranded cassettes encoding the zinc finger modules and relevant leader, linker, and terminator sequences are generated by PCR according to the procedure described below, using the appropriate oligonucleotide templates of Table 4, and primers of Table 5.

TABLE 4

Nucleotide sequences encoding zinc finger
modules and other peptide sequences used
in the construction of 3-finger proteins.

| | | | | |
|---|---|---|---|---|
| | | | SEQ | |
| x | CODE | FINGER | ID NO | NUCLEOTIDE SEQUENCE |

| | | | | |
|---|---|---|---|---|
| 1 | AS144 | ZIF268 F1 | 1990 | TATGCGTGCCCGGTGGAAAGCTGC |
| | | | | GATCGTCGTTTTAGCCGTAGCGAT |
| | | | | GAACTGACCCGTCATATTCGTATT |
| | | | | CAT |
| 2 | AS145 | ZIF268 F2 | 1991 | TTTCAGTGCCGTATTTGCATGCGT |
| | | | | AACTTTAGCCGTAGCGATCATCTG |
| | | | | AGCACCCATATTCGTACCCAT |
| 3 | AS148 | Sp1 F3 | 1992 | TTTAGCTGCCCGATTTGCGAAAAA |
| | | | | CGTTTTATGCGTAGCGATCATCTG |
| | | | | ACCAAACATGCGCGTCGTCAT |
| 4 | AS149 | WT1 F1 | 1993 | TTTATGTGCGCGTATCCGGGCTGC |
| | | | | AACAAACGTTATTTTAAACTGAGC |
| | | | | CATCTGCAGatgCATAGCCGTAAA |
| | | | | CAT |
| 5 | AS150 | O15391 | 1994 | TTTGTGTGCCCGTTTGATGTGTGC |
| | | | | AACCGTAAATTTGCGCAGAGCACC |
| | | | | AACCTGAAAACCCATATTCTGACC |
| | | | | CAT |
| 6 | AS151 | O75626 | 1995 | TTTAAATGCCAGACCTGCAACAAA |
| | | | | GGCTTTACCCAGCTGGCGCATCTG |
| | | | | CAGAAACATTATCTGGTGCAT |
| 7 | AS152 | ZN45-AAA | 1996 | TATAAATGCGAAGAATGCGGCAAA |
| | | | | GGCTTTAGCCAGGCGAGCAACCTG |
| | | | | CTGGCGCATCAGCGTGGCCAT |
| 8 | AS153 | Z165-GCC | 1997 | TATGAATGCAACGAATGCGGCAAA |
| | | | | AGCTTTGCGGAAAGCAGCGATCTG |
| | | | | ACCCGTCATCGTCGTATTCAT |
| 9 | | MAEERP | 1998 | ATGGCGGAAGAACGTCCG |
| | | leader | | |
| 10 | | TGEKP | 1999 | ACCGGCGAAAAACCG |
| | | linker | | |
| 11 | | G₃S-HA- | 2000 | CATCTGCGCCAGAAGGACGGCGGC |
| | | tag (tag) | | GGCAGCTATCCGTATGATGTGCCG |
| | | | | GATTATGCGTAA |

TABLE 5

Modifying oligonucleotides used
for mini-library construction.

| | | | SEQ | |
|---|---|---|---|---|
| x | CODE | NAME | ID NO | SEQUENCE |
| 1 | AS5 | pETFwd1 | 2001 | CGCTGACTTCCGCGTTTCC |
| 2 | AS86 | SDRev | 2002 | ATGTATATCTCCTTCTTAAAGTT |
| 3 | AS93 | ZnF1Fwd | 2003 | AACTTTAAGAAGGAGATATACATA |
| | | | | TGGCGGAAGAACGTCCGTATGCGT |
| | | | | GCCCGGTGGAAAG |
| 4 | AS94 | ZnF2Fwd | 2004 | AACTTTAAGAAGGAGATATACATA |
| | | | | TGGCGGAAGAACGTCCGTTTCAGT |
| | | | | GCCGTATTTGCATG |
| 5 | AS95 | ZnF3Fwd | 2005 | AACTTTAAGAAGGAGATATACATA |
| | | | | TGGCGGAAGAACGTCCGTTTAGCT |
| | | | | GCCCGATTTGCG |
| 6 | AS96 | ZnF4Fwd | 2006 | AACTTTAAGAAGGAGATATACATA |
| | | | | TGGCGGAAGAACGTCCGTTTATGT |
| | | | | GCGCGTATCCGGG |
| 7 | AS97 | ZnF5Fwd | 2007 | AACTTTAAGAAGGAGATATACATA |
| | | | | TGGCGGAAGAACGTCCGTTTATGT |
| | | | | GCGCGTATCCGGG |
| 8 | AS98 | ZnF6Fwd | 2008 | AACTTTAAGAAGGAGATATACATA |
| | | | | TGGCGGAAGAACGTCCGTTTAAAT |
| | | | | GCCAGACCTGCAAC |
| 9 | AS99 | ZnF7Fwd | 2009 | AACTTTAAGAAGGAGATATACATA |
| | | | | TGGCGGAAGAACGTCCGTATAAAT |
| | | | | GCGAAGAATGCGGC |
| 10 | AS100 | ZnF8Fwd | 2010 | AACTTTAAGAAGGAGATATACATA |
| | | | | TGGCGGAAGAACGTCCGTATGAAT |
| | | | | GCAACGAATGCGGC |
| 11 | AS101 | 1Link1Rev | 2011 | CGGTTTTTCGCCGGTATGAATACG |
| | | | | AATATGACGGG |
| 12 | AS102 | 1Link2Rev | 2012 | CGGTTTTTCGCCGGTATGGGTACG |
| | | | | AATATGGGTGC |
| 13 | AS103 | 1Link3Rev | 2013 | CGGTTTTTCGCCGGTATGACGACG |
| | | | | CGCATGTTTGG |
| 14 | AS104 | 1Link4Rev | 2014 | CGGTTTTTCGCCGGTATGTTTACG |
| | | | | GCTATGCATCTG |
| 15 | AS105 | 1Link5Rev | 2015 | CGGTTTTTCGCCGGTATGGGTCAG |
| | | | | AATATGGGTTTTC |
| 16 | AS106 | 1Link6Rev | 2016 | CGGTTTTTCGCCGGTATGCACCAG |
| | | | | ATAATGTTTCTGC |
| 17 | AS107 | 1Link7Rev | 2017 | CGGTTTTTCGCCGGTATGGCCACG |
| | | | | CTGATGCGC |
| 18 | AS108 | 1Link8Rev | 2018 | CGGTTTTTCGCCGGTATGAATACG |
| | | | | ACGATGACGGG |
| 19 | AS109 | 1Link1Fwd | 2019 | CATACCGGCGAAAAACCGTATGCG |
| | | | | TGCCCGGTGGAAAG |
| 10 | AS110 | 1Link2Fwd | 2020 | CATACCGGCGAAAAACCGTTTCAG |
| | | | | TGCCGTATTTGCATG |
| 11 | AS111 | 1Link3Fwd | 2021 | CATACCGGCGAAAAACCGTTTAGC |
| | | | | TGCCCGATTTGCG |
| 12 | AS112 | 1Link4Fwd | 2022 | CATACCGGCGAAAAACCGTTTATG |
| | | | | TGCGCGTATCCGGG |
| 13 | AS113 | 1Link5Fwd | 2023 | CATACCGGCGAAAAACCGTTTGTG |
| | | | | TGCCCGTTTGATGTG |
| 14 | AS114 | 1Link6Fwd | 2024 | CATACCGGCGAAAAACCGTTTAAA |
| | | | | TGCCAGACCTGCAAC |
| 15 | AS115 | 1Link7Fwd | 2025 | CATACCGGCGAAAAACCGTATAAA |
| | | | | TGCGAAGAATGCGGC |
| 16 | AS116 | 1Link8Fwd | 2026 | CATACCGGCGAAAAACCGTATGAA |
| | | | | TGCAACGAATGCGGC |
| 17 | AS117 | 2Link1Rev | 2027 | TGGCTTCTCACCCGTGTGATGAAT |
| | | | | ACGAATATGACGGGTC |
| 18 | AS118 | 2Link2Rev | 2028 | TGGCTTCTCACCCGTGTGATGGGT |
| | | | | ACGAATATGGGTGC |
| 19 | AS119 | 2Link3Rev | 2029 | TGGCTTCTCACCCGTGTGATGACG |
| | | | | ACGCGCATGTTTGG |
| 20 | AS120 | 2Link4Rev | 2030 | TGGCTTCTCACCCGTGTGATGTTT |
| | | | | ACGGCTATGCATCTG |
| 21 | AS121 | 2Link5Rev | 2031 | TGGCTTCTCACCCGTGTGATGGGT |
| | | | | CAGAATATGGGTTTTC |
| 22 | AS122 | 2Link6Rev | 2032 | TGGCTTCTCACCCGTGTGATGCAC |
| | | | | CAGATAATGTTTCTGC |
| 23 | AS123 | 2Link7Rev | 2033 | TGGCTTCTCACCCGTGTGATGGCC |
| | | | | ACGCTGATGCGC |
| 24 | AS124 | 2Link8Rev | 2034 | TGGCTTCTCACCCGTGTGATGAAT |
| | | | | ACGACGATGACGGG |
| 25 | AS125 | 2Link1Fwd | 2035 | CACGGGTGAGAAGCCATATGCGTG |
| | | | | CCCGGTGGAAAG |
| 26 | AS126 | 2Link2Fwd | 2036 | CACGGGTGAGAAGCCATTTCAGTG |
| | | | | CCGTATTTGCATG |
| 27 | AS127 | 2Link3Fwd | 2037 | CACGGGTGAGAAGCCATTTAGCTG |
| | | | | CCCGATTTGCG |
| 28 | AS128 | 2Link4Fwd | 2038 | CACGGGTGAGAAGCCATTTATGTG |
| | | | | CGCGTATCCGGG |
| 29 | AS129 | 2Link5Fwd | 2039 | CACGGGTGAGAAGCCATTTGTGTG |
| | | | | CCCGTTTGATGTG |
| 30 | AS130 | 2Link6Fwd | 2040 | CACGGGTGAGAAGCCATTTAAATG |
| | | | | CCAGACCTGCAAC |
| 31 | AS131 | 2Link7Fwd | 2041 | CACGGGTGAGAAGCCATATAAATG |
| | | | | CGAAGAATGCGGC |
| 32 | AS132 | 2Link8Fwd | 2042 | CACGGGTGAGAAGCCATATGAATG |
| | | | | CAACGAATGCGGC |
| 33 | AS133 | 3HA1Rev | 2043 | CTAGGAATTCTTACGCATAATCCG |
| | | | | GCACATCATACGGATAGCTGCCGC |
| | | | | CGCCGTCCTTCTGGCGCAGATGAA |
| | | | | TACGAATATGACGGGTC |
| 34 | AS134 | 3HA2Rev | 2044 | CTAGGAATTCTTACGCATAATCCG |
| | | | | GCACATCATACGGATAGCTGCCGC |
| | | | | CGCCGTCCTTCTGGCGCAGATGGG |
| | | | | TACGAATATGGGTGC |
| 35 | AS135 | 3HA3Rev | 2045 | CTAGGAATTCTTACGCATAATCCG |
| | | | | GCACATCATACGGATAGCTGCCGC |
| | | | | CGCCGTCCTTCTGGCGCAGATGAC |
| | | | | GACGCGCATGTTTGG |
| 36 | AS136 | 3HA4Rev | 2046 | CTAGGAATTCTTACGCATAATCCG |
| | | | | GCACATCATACGGATAGCTGCCGC |
| | | | | CGCCGTCCTTCTGGCGCAGATGTT |
| | | | | TACGGCTATGCATCTG |
| 37 | AS137 | 3HA5Rev | 2047 | CTAGGAATTCTTACGCATAATCCG |
| | | | | GCACATCATACGGATAGCTGCCGC |
| | | | | CGCCGTCCTTCTGGCGCAGATGGG |
| | | | | TCAGAATATGGGTTTTC |
| 38 | AS138 | 3HA6Rev | 2048 | CTAGGAATTCTTACGCATAATCCG |
| | | | | GCACATCATACGGATAGCTGCCGC |
| | | | | CGCCGTCCTTCTGGCGCAGATGCA |
| | | | | CCAGATAATGTTTCTGC |
| 39 | AS139 | 3HA7Rev | 2049 | CTAGGAATTCTTACGCATAATCCG |
| | | | | GCACATCATACGGATAGCTGCCGC |
| | | | | CGCCGTCCTTCTGGCGCAGATGGC |
| | | | | CACGCTGATGCGC |
| 40 | AS140 | 3HA8Rev | 2050 | CTAGGAATTCTTACGCATAATCCG |
| | | | | GCACATCATACGGATAGCTGCCGC |
| | | | | CGCCGTCCTTCTGGCGCAGATGAA |
| | | | | TACGACGATGACGGG |
| 41 | AS141 | Rev3 | 2051 | CTAGGAATTCTTACGCATAATC |
| 42 | AS142 | 1LinkRev | 2052 | CGGTTTTTCGCCGGTATG |
| 43 | AS143 | 2LinkRev | 2053 | TGGCTTCTCACCCGTGTG |

1. Library 1.

Once made into double stranded DNA cassettes, the finger units are attached to T7 upstream expression sequences by PCR overlap extension, using the following protocol.

(a) Upstream sequences are first extracted from pET23a by PCR using primers pETFwd1 and SDRev, generating the fragment pET5′.

(b) The fingers for cassette A are amplified with forward primers ZnFxFwd (AS93-100) and reverse primers 1LinkxRev (AS01-AS10S), where x is the number of a particular finger from Tables 3 and 4, as indicated.

(c) The fingers for cassette B are amplified with forward primers 1LinkxFwd (AS109-116) and reverse primers 2LinkxRev (AS117-AS 124), where x refers to the finger module number.

(d) The fingers for cassette C are amplified with forward primers 2LinkxFwd (AS125-132) and reverse primers 3HAxRev (AS133-AS140), where x refers to the appropriate zinc finger module.

The steps to create cassettes A, B and C are performed separately, however, mixed populations of template oligonucleotides can be added to each PCR of steps (a), (b), and (c) to produce a library of each cassette.

The final 3-finger library is assembled by overlap extension as outlined in FIG. 2. In the first step the mixed pool of cassette A is appended to the upstream sequences, pET5′.

Equimolar amounts are mixed and PCR-cycled in the absence of primers. The reaction product is either purified immediately or reamplified before purification using primers pETFwd1 and 1LinkRev.

In the second step cassette B (mixed pool) is appended to the product of the above step. Again, equimolar amounts are mixed and PCR-cycled in the absence of primers. The reaction product is either purified immediately or reamplified before purification using primers pETFwd1 and 2LinkRev.

In the final step cassette C (mixed pool) is appended to the above product. Equimolar amounts are mixed and PCR-cycled in the absence of primers. As before, the reaction product may be purified immediately or reamplified before purification using primers pETFwd1 and Rev3. (see, also FIG. 2).

2. Library 2.

Library 2 is assembled in a similar maimer to Library 1 except that cassette A is represented by Zif268 finger 1 only.

The final PCR products containing T7 promoter sequences and encoding 3-finger peptides attached to an HA-antibody tag are purified and used for the production of protein.

b. Zinc Finger Library Screening.

Two exemplary methods for screening zinc finger libraries, such as those produced above, are described in Protocol A and Protocol B, below.

Protocol A:

The peptides of library 1 and library 2 are screened to select 3-zinc finger domains which bind the sequences: 5′-GCG-TGG-GCG-3′; 5′-GGA-TAA-GCG-3′; and 5′-GCC-GAG-TGG-3′. Since library 2 contains Zif268 finger 1 in the N-terminal position, in theory, these peptides should only bind the sequences, 5′-GCG-TGG-GCG-3′, and 5′-GGA-TAA-GCG-3′. Hence, library 2 is effectively used to select 2-finger units which bind strongest to the 6 bp sequences, 5′-GCG-TGG-3′, and 5′-GGA-TAA-3′. Double stranded binding sites for use in the selection protocol are generated by annealing the complimentary oligonucleotides: Zif.b site and Zif site RC (AS154 and AS155); #1#5#6.b and #1#5#6 RC (AS156 and AS157); and #2#4#8.b and #2#4#8 RC (AS158 and AS159). The top strand of each binding site is biotinylated, allowing capture of binding site/zinc finger/HA-antibody ternary complexes to the streptavidin-coated plate in an ELISA screening assay. The oligonucleotides are displayed in Table 6, below.

TABLE 6

Oligonucleotide sequences used
to generate double stranded binding
sites used in the selection procedure.

| | | | | |
|---|---|---|---|---|
| x | Code | Name | SEQ ID NO | Sequence |

| | | | | |
|---|---|---|---|---|
| 1 | AS154 | Zif.b site | 2054 | TTTTTTTTTTGCGTGGGCGTTT |
| | | | | TTTTTTT |
| 2 | AS155 | Zif site RC | 2055 | AAAAAAAAAACGCCCACGCAAA |
| | | | | AAAAAAA |
| 3 | AS156 | #1#5#6.b | 2056 | TTTTTTTTTTGGATAAGCGTTT |
| | | | | TTTTTTT |
| 4 | AS157 | #1#5#6 RC | 2057 | AAAAAAAAAACGCTTATCCAAA |
| | | | | AAAAAAA |
| 5 | AS158 | #2#4#8.b | 2058 | TTTTTTTTTGCCTGTTGGTTTT |
| | | | | TTTTTTT |
| 6 | AS159 | #2#4#8 RC | 2059 | AAAAAAAAAAACCAACAGGCAA |
| | | | | AAAAAAA |

The PCR-amplified 3-finger constructs are gel-purified from a 1% TAE-agarose gel using the Gel Extraction Kit (Qiagen) and quantified based on absorbance at 260 nM. Dilutions (in 0.25 mg/ml λ DNA) of DNA template encoding for either library 1 or 2 are prepared at the final total template concentration of 4.2 fM and 1 fM, respectively. At these concentrations 1 μl of template contains approximately 2500 and 600 molecules of library 1 or library 2, respectively. At such low concentrations, such samples must be PCR amplified to generate enough template for protein expression. Hence, these 1 μl aliquots are taken and added to 1 ml PCR pre-mix, containing primers Rev3 (AS141) and pETFwd2 (primer sequences shown below, see Table 7). The PCR pre-mixes are then aliquoted into 96 (or 384) well plates at 10 μl per well, which is the equivalent of approximately 25 or 6 molecules of library 1 or library 2 template, respectively. Templates are amplified using 30 cycles of PCR. After this first round of PCR, 0.5 μl aliquots of PCR product are added to new 10 μl PCR pre-mixes (in 96 or 384 well format), containing nested primers, pETFwd3 and Rev3, and amplified for another 30 cycles. The resultant product is concentrated enough to perform in vitro transcription/translation.

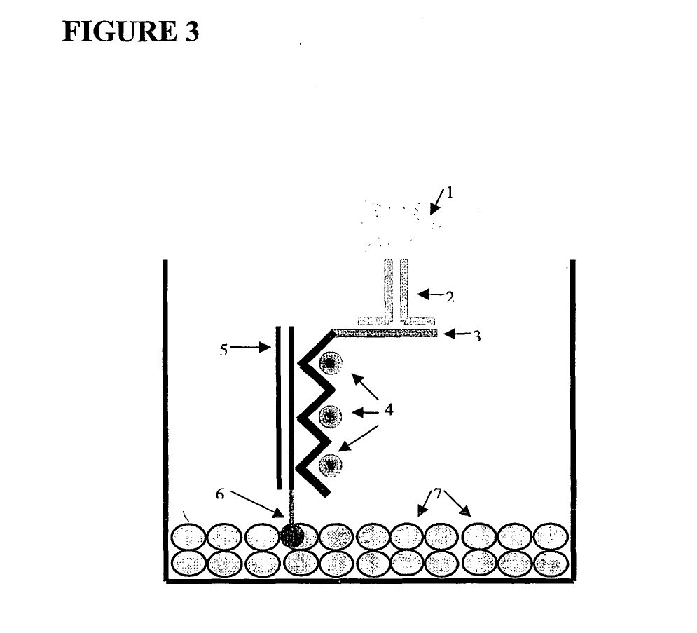

In vitro translation experiments using TNT PCR coupled transcription-translation mix (Promega) are assembled according to the manufacturer's instructions. Typically 5 μl final volume contains 1 μl of each PCR product and 4 μl rabbit reticulocyte pre-mix (containing 20 μM methionine, 12.5 μg/ml λ Hind III digest (Roche), 500 μM ZnCl₂ (Sigma), 0.7 μl H₂O, 40 nM PCR-amplified DNA template). Reactions are incubated at 30° C. for 90 minutes. 50 μl PBS binding buffer containing 0.1% BSA (Sigmna), 0.5% Tween 20 (Sigma), 50 μM ZnCl₂, 10 nM of the appropriate biotinylated binding site, 25 μU/ml rat 3F10 anti-HA HRP conjugate (Roche) is added to the translation mix and incubated for 45 minutes at room temperature. The binding mix is thereafter transferred to pre-blocked black streptavidin-coated 8-well strips or 96/384 well plates (Roche), and the ternary complexes containing 3-finger peptide, biotinylated binding site and anti-HA HRP antibody are captured while shaking at 200 rpm for 45 minutes at room temperature. The wells are then washed five times with 100 μl PBS binding buffer containing 0.1% BSA (Sigma), 0.5% Tween 20 (Sigma), 50 μM ZnCl₂ to remove unbound components. Finally, the retained HRP activity is measured by adding 50 μl QuantaBlu fluorogenic HRP substrate (Pierce). FIG. 3 demonstrates the capture and detection of target site-binding zinc finger peptides using the assay described. Fluorescence is measured on a SpectraMax Gemini XS (Molecular Devices) fluorescence microplate reader at 320 nm excitation, 433 nm emission and 420 nm cut-off values.

The wells that give the highest levels of fluorescence are those which contain the highest number of, or tightest binding 3-finger peptides. PCR products from the second PCR amplification stage, corresponding to such samples, are purified from TAE-agarose gels and quantified, as above. Pure PCR products are diluted to approximately 50 molecules per μl (which is equivalent to approximately 100 aM concentration) in 0.25 mg/ml λ DNA. As above, 1 μl samples of template are added to 1 ml PCR pre-mix containing primers, pETFwd4 and Rev3. 10 μl aliquots are placed in each well of a 96 well plate. At this stage, there is (on average) 0.5 template molecules per aliquot. Therefore, generally speaking, half of the samples will contain no template and half will contain a single template molecule. Samples are then PCR amplified using 30 cycles. Again, 0.5 μl PCR samples are taken from each well and amplified again by 30 cycles of PCR using the nested primers, pETFwd5 and Rev3. 1 μl of each of these PCR products is used for protein expression, as described above. At this stage, the highest levels of fluorescence correspond to the samples containing the tightest binding 3-finger peptides. The PCR product encoding such peptides is purified, as before, and can be sequenced to determine the protein sequence of the optimal 3-zinc finger domain for the appropriate binding site.

If further rounds of selection are required, PCR amplification can be conducted with the nested primers pETFwd6, pETFwd9 and pETFwd7, also shown below (Table 7).

TABLE 7

Primers used for PCR amplification of
3-finger cassettes (as constructed by the
procedure of FIG. 2) to provide template
used in screening zinc finger libraries.

| NAME | SEQ ID NO | SEQUENCE |
|---|---|---|
| pETFwd1 | 2060 | CGCTGACTTCCGCGTTTCC |
| pETFwd2 | 2061 | TCCAGACTTTACGAAACACGG |
| pETFwd3 | 2062 | CGAAGACCATTCATGTTGTTGC |
| pETFwd4 | 2063 | GTCGCAGACGTTTTGCAGC |
| pETFwd5 | 2064 | GCAGTCGCTTCACGTTCGC |
| pETFwd6 | 2065 | CGCTCGCGTATCGGTGATTC |
| pETFwd9 | 2066 | CATTCTGCTAACCAGTAAGGC |
| pETFwd7 | 2067 | GCCTAGCCGGGTCCTCAAC |

Protocol B:

The peptides of library 2 were screened to select 3-zinc finger domains which bind the sequences: 5′-GCG-TGG-GCG-3′, and 5′-GGG-AGG-CCT-3′. Double stranded binding sites for use in the selection protocol were generated by annealing the complementary oligonucleotides: Zif.b site and Zif site RC (AS154 and AS155, shown above), which generated the 5′-GCG-TGG-GCG-3′ binding site; and the oligonucleotides 5′-TTTTTTTTTTGGGAGGCCTTTTTTTTTT-3′ (SEQ ID NO:2123) and 5′-AAAAAAAAAAAGGCCTCCCAAAAAAAAAA-3′ (SEQ ID NO:2124), which generated the 5′-GGG-AGG-CCT-3′ binding site. The top strand of each binding site was biotinylated, allowing capture of binding site/zinc finger/HA-antibody ternary complexes onto streptavidin-coated plate in an ELISA screening assay.

The 3-finger library 2 constructs were cloned into the multiple cloning site of vector pET23a (Novagen), using appropriate restriction sites. This library was then transformed into E.coli and plated out to grow single colonies. 384 colonies (which should represent the vast majority of the 64 member library) were picked into 2xYT media with ampicillin and cultures grown at 37° C. overnight. Library 2 expression cassettes were recovered from bacteria by PCR using primers pETFwdx (where x is 1-7, eg pETFwd1) and Rev3 as described in Protocol A above.

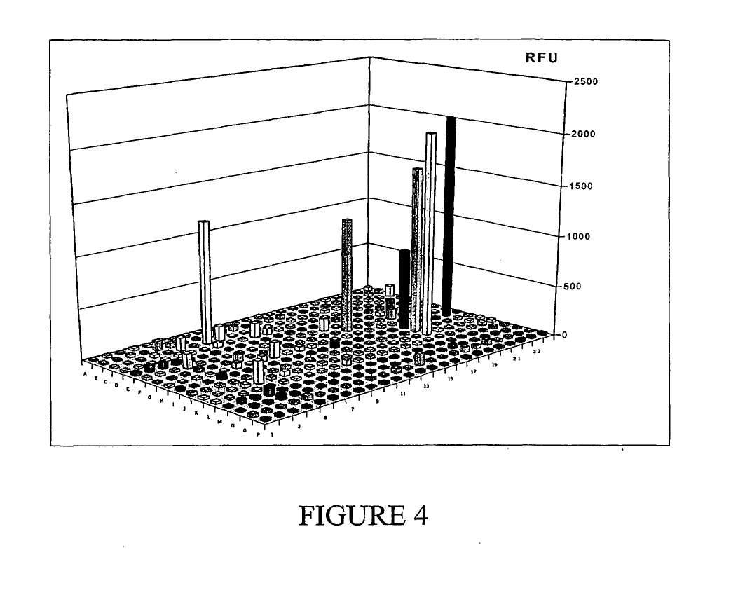

In vitro coupled transcription/translation of PCR products was conducted as described above, with the difference that each of the 384 zinc finger peptides was screened individually in a well of a 384 well plate. The library was screened against the 5′-GCG-TGG-GCG-3′, and 5′-GGG-AGG-CCT-3′ binding sites, as detailed in Protocol A. Wells that yielded the highest levels of fluorescence were those which contain the tightest binding 3-finger peptides. The ELISA results from the screen of the 384 samples against the 5′-GCG-TGG-GCG-3′ site are shown in FIG. 4. Six constructs displayed significant binding to the target site and these are termed C8, G16, I19, I23, J19 and K19 according to their coordinates on the 384-well plate. Similarly, one construct (B10) showed strong binding to the 5′-GGG-AGG-CCT-3′ target site. PCR products encoding the tightest binding peptides can be purified, as described supra, and sequenced.

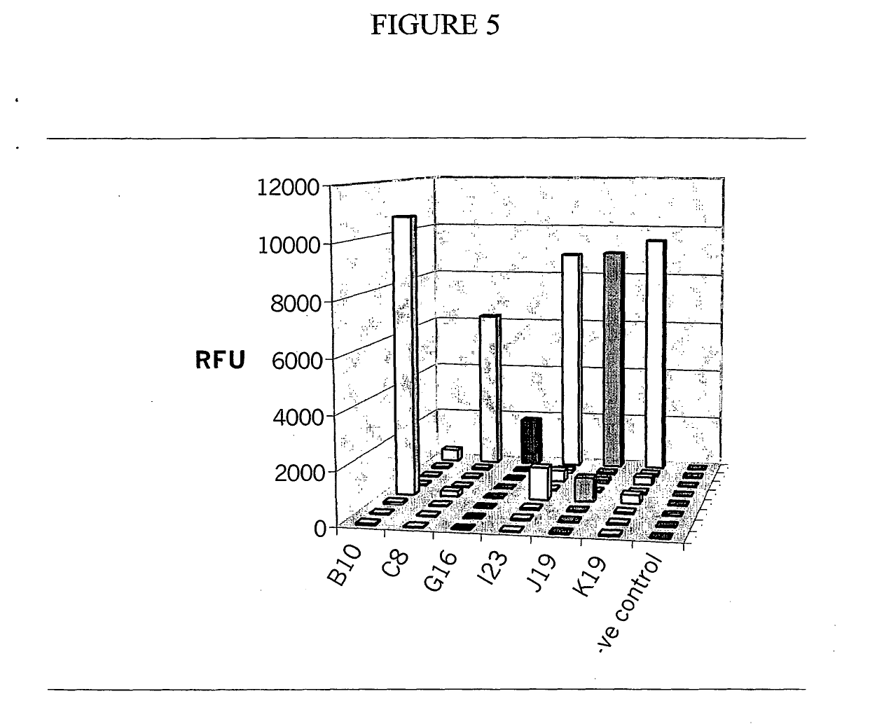

Some of the selected constructs: CS, J19, K19, I23, G16 (which bind the 5′-GCG-TGG-GCG-3′ site) and B10 (which binds the 5′-GGG-AGG-CCT-3′ site), were selected and screened against a range of different binding sites to test their specificity. The sites used were: 5′-GCG-TGG-GCG-3′; 5′-CCA-CTC-GGC-3′; 5′-CCT-AGG-GGG-3′; 5′-GGA-TAA-GCG-3′; 5′-GGG-AGG-CCT-3′; 5′-GCG-TAA-GGA-3′; and 5′-GCG-GGG-GGA-3′. The binding assay was conducted as described above. The results (FIG. 5) show that the selected 3-zinc finger peptides bind preferentially to their target site, in comparison to the alternative binding sites tested.

### Example 5

### Human Zinc Finger Module Libraries for Rapid Selection of 2-Finger Units

The preferred subunits of a poly-zinc finger construction strategy are in the form of two-finger sub-domains. Assuming that there are 1,000 individual natural finger modules, a library of all combinations of such zinc finger modules, in 2-finger units, would contain 1,000,000 members. All of the 1,000 natural finger modules would have to be made from oligonucleotides, and the expense would be considerable. Furthermore, this figure is likely to be an underestimate of the number of natural fingers. Hence, due to the huge numbers of natural, human zinc finger modules available, it is advantageous to limit the size of the libraries screened, as discussed in the Description. One way in which library size can be reduced is to limit the library members to zinc finger modules which are predicted to bind the desired sequence. For instance, based on the target sites in Example 1, if 2-finger domains are required to bind the sequence 5′-GCG-TGG-3′, an individual library can be constructed from the zinc finger modules predicted to bind the sequences 5′-GCG-3′ and 5′-TGG-3′. Equally, if the sequence 5′-GGA-TAA-3′ is to be targeted, zinc finger modules predicted to bind the sequences and 5′-GGA-3′ and 5′-TAA-3′ can be used. Table 8 shows the natural, human zinc finger modules from Example 1, which are predicted to bind the aforementioned 3 bp sequences.

TABLE 8
The natural, human zinc finger modules predicted to bind the sequences 5′-GCG-3′,
5′-TGG-3′, 5′-GGA-3′ and 5′-TAA-3′.

| 5′-GCG-3′ | 5′-TGG-3′ | 5′-GGA-3′ | 5′-TAA-3′ |
|---|---|---|---|
| Zif268 finger 1 (GCG) | Zif268 finger 2 (TGG) | BCL6 (NGA) | TYY1 (NAA) |
| Zif268 finger 3 (GCG) | MAZ finger 2 (TGG) | O75626 (GGA) | O15391 (YAA) |
| Sp1 finger 2 (GCG) | WT1 finger 3 (TGG) | ZN45 (N^{N}/_{T}A) | O75626 (YAA) |
| WT1 finger 4 (GCG) | SP4 (NGG) | O15535 (GNA) | ZN45 (N^{N}/_{T}A) |
| BTE1 (GCG) | BTE1 (NGG) | Q15776 (GNA) | Z136 (TNN) |
| O43296 (GNG) | Z136 (TNN) | O60893 (GNA) | Z239 (YAA) |
| Z174 (GCG, RNA) | Q15776 (NGG) | Z132 (a) (GGA) | Q15776 (a) (TNA) |
| Z202 (GCG, RNA) | ZN84 (YGG) | Z132 (b) (GGA) | Q15776 (b) (TNA) |
| | | Z132 (GGN) | Z195 (YAA) |
| | | ZN85 (GGA) | ZN84 (YAA) |
| | | | O75346 (TAA) |
| | | | ZN43 (TAA) |

On the basis of the specificities shown in Table 5, a library of 2-finger units to target the 6 bp sequence 5′-GCG-TGG-3.′ has 64 (8×8) members, and a library to target the sequence 5′-GGA-TAA-3′ has 120 (10×12) members. To screen sample sizes of this magnitude we can construct each 2-finger unit specifically (using for example, an 8×8 or 10×12 matrix arrangement), and assay the samples containing individual clones using the fluorescent-ELISA protocol of Example 4. Such a procedure can save time in comparison to constructing all possible 64 or 120 variants in a random fashion (as a library), as described in Example 4, because the number of constructs screened would have to be considerably higher.

a. Construction of 2-Finger Domains to Bind 5′-GCG-TGG-3′

A 64 member, 2-finger library is constructed from the natural, human zinc finger modules predicted to bind the sequences 5′-GCG-3′ and 5′-TGG-3′ (Table 8, columns 1 and 2).

The 2-finger library units are all attached to the C-terminus of Zif268 finger 1, which acts as an anchor finger. The construction protocol is different from that described in Example 4, as described below.

Zinc Finger Cassettes

Nucleotide sequences encoding the amino acid sequences of the 16 zinc finger modules (Table 8, columns 1 and 2) are determined, taking into account human codon preferences, and the corresponding nucleotide sequences are synthesised as single stranded oligonucleotides, shown in Table 9. Double stranded cassettes encoding the zinc finger modules and flanking linker sequences are generated by PCR using the appropriate primers, shown in Table 10.

TABLE 9

Nucleotide sequences of zinc finger modules
and nucleotide sequences encoding other peptide
sequences used in the construction of peptides
to bind the sequence 5′-GCG-TGG-3′.

| | | SEQ | |
|---|---|---|---|
| X | FINGER | ID NO | NUCLEOTIDE SEQUENCE |
| 1 | Zif268 F1 | 2068 | TACGCCTGCCCCGTGGAGAGCTGCGACCGCC |
| | | | GCTTCAGCCGCAGCGACGAGCTGACCCGCCA |
| | | | CATCCGCATCCAC |
| 2 | Zif268 F3 | 2069 | TTCGCCTGCGACATCTGCGGCCGCAAGTTCG |
| | | | CCCGCAGCGACGAGCGCAAGCGCCACACCAA |
| | | | GATCCAC |
| 3 | Sp1 F2 | 2070 | TTCGCCTGCAGCTGGCAGGACTGCAACAAGA |
| | | | AGTTCGCCCGCAGCGACGAGCTGGCCCGCCA |
| | | | CTACCGCACCCAC |
| 4 | WT1 F4 | 2071 | TTCAGCTGCCGCTGGCCCAGCTGCCAGAAGA |
| | | | AGTTCGCCCGCAGCGACGAGCTGGTGCGCCA |
| | | | CCACAACATGCAC |
| 5 | BTE1 | 2072 | TTCCCCTGCACCTGGCCCGACTGCCTGAAGA |
| | | | AGTTCAGCCGCAGCGACGAGCTGACCCGCCA |
| | | | CTACCGCACCCAC |
| 6 | O43296 | 2073 | TACGAGTGCGTGGAGTGCGGCAAGGCCTTCA |
| | | | CCCGCATGAGCGGCCTGACCCGCCACAAGCG |
| | | | CATCCAC |
| 7 | Z174 | 2074 | TACAAGTGCGACGACTGCGGCAAGAGCTTCA |
| | | | CCTGGAACAGCGAGCTGAAGCGCCACAAGCG |
| | | | CGTGCAC |
| 8 | Z202 | 2075 | TACCGCTGCGACGACTGCGGCAAGCACTTCC |
| | | | GCTGGACCAGCGACCTGGTGCGCCACCAGCG |
| | | | CACCCAC |
| 9 | Zif268 F2 | 2076 | TTCCAGTGCCGCATCTGCATGCGCAACTTCA |
| | | | GCCGCAGCGACCACCTGAGCACCCACATCCG |
| | | | CACCCAC |
| 10 | MAZ F2 | 2077 | TACAACTGCAGCCACTGCGGCAAGAGCTTCA |
| | | | GCCGCCCCGACCACCTGAACAGCCACGTGCG |
| | | | CCAGGTGCAC |
| 11 | WT1 F3 | 2078 | TTCCAGTGCAAGACCTGCCAGCGCAAGTTCA |
| | | | GCCGCAGCGACCACCTGAAGACCCACACCCG |
| | | | CACCCAC |
| 12 | Sp4 | 2079 | CACAAGTGCCCCTACAGCGGCTGCGGCAAGG |
| | | | TGTACGGCAAGAGCAGCCACCTGAAGGCCCA |
| | | | CTACCGCGTGCAC |
| 13 | BTE1 | 2080 | CACAAGTGCCCCTACAGCGGCTGCGGCAAGG |
| | | | TGTACGGCAAGAGCAGCCACCTGAAGGCCCA |
| | | | CTACCGCGTGCAC |
| 14 | Z136 | 2081 | TTCGAGTGCAAGCGCTGCGGCAAGGCCTTCC |
| | | | GCAGCAGCAGCAGCTTCCGCCTGCACGAGCG |
| | | | CACCCAC |
| 15 | Q15776 | 2082 | TACGAGTGCGACGAGTGCGGCAAGACCTTCC |
| | | | GCCGCAGCAGCCACCTGATCGGCCACCAGCG |
| | | | CAGCCAC |
| 16 | ZN84 | 2083 | TACGAGTGCGGCGAGTGCGGCAAGGCCTTCA |
| | | | GCCGCAAGAGCCACCTGATCAGCCACTGGCG |
| | | | CACCCAC |

¹ RNA Binding.

The primers used to amplify the N-terminal finger of the pair (the equivalent of cassette B, above) add TGEKP (SEQ ID NO:3) linker sequences, and the restriction site XmaI (5′-CCC-GGG-3′) at the 5′ end and an AgeI site (5′-ACC-GGT-3′) at the 3′ end. AgeI and XmaI create compatible ends, but have unique restriction sites. These primers are called CasBxFwd and CasBxRev, respectively, where x refers to the number of the zinc finger module in Table 9. The primers used to amplify the C-terminal finger of the pair (the equivalent of cassette C, above) add TGEKP (SEQ ID NO:3) linker sequences, and the restriction site XmaI at the 5′ end and ‘a sequence encoding LRQKDGGGS (SEQ ID NO:2125), containing a restriction site for BamHI at the 3′ end. These primers are referred to as CasCxFwd and CasCxRev, respectively. The 16 individual zinc finger cassettes are then purified using the QIAquick PCR purification kit (Qiagen).

TABLE 10

Oligonucleotides used for PCR construction of rapid zinc
finger library. Annealing sequences are shown in bold,
restriction sites are underlined.

| | | |
|---|---|---|
| Name | SEQ ID NO | Sequence |

| | | |
|---|---|---|
| CasB9Fwd | 2084 | GATCCCCGGGGAGAAGCCCTTCCAGTGCCGCATCTGCAT |
| CasB10Fwd | 2085 | GATCCCCGGGGAGAAGCCCTACAACTGCAGCCACTGCGG |
| CasB11Fwd | 2086 | GATCCCCGGGGAGAAGCCCTTCCAGTGCAAGACCTGCCA |
| CasB12Fwd | 2087 | GATCCCCGGGGAGAAGCCCCACAAGTGCCCCTACAGCG |
| CasB13Fwd | 2088 | GATCCCCGGGGAGAAGCCCCACAAGTGCCCCTACAGCG |
| CasB14Fwd | 2089 | GATCCCCGGGGAGAAGCCCTTCGAGTGCAAGCGCTGCG |
| CasB15Fwd | 2090 | GATCCCCGGGGAGAAGCCCTACGAGTGCGACGAGTGCG |
| CasB16Fwd | 2091 | GATCCCCGGGGAGAAGCCCTACGAGTGCGGCGAGTGCG |
| CasC1Fwd | 2092 | GATCCCCGGGGAGAAGCCCTACGCCTGCCCCGTGGAG |
| CasC2Fwd | 2093 | GATCCCCGGGGAGAAGCCCTTCGCCTGCGACATCTGCG |
| CasC3Fwd | 2094 | GATCCCCGGGGAGAAGCCCTTCGCCTGCAGCTGGCAGG |
| CasC4Fwd | 2095 | GATCCCCGGGGAGAAGCCCTTCAGCTGCCGCTGGCCC |
| CasC5Fwd | 2096 | GATCCCCGGGGAGAAGCCCTTCCCCTGCACCTGGCCC |
| CasC6Fwd | 2097 | GATCCCCGGGGAGAAGCCCTACGAGTGCGTGGAGTGCG |
| CasC7Fwd | 2098 | GATCCCCGGGGAGAAGCCCTACAAGTGCGACGACTGCGG |
| CasC8Fwd | 2099 | GATCCCCGGGGAGAAGCCCTACCGCTGCGACGACTGCG |
| CasB9Rev | 2100 | CTTCTCACCGGTGTGGGTGCGGATGTGGGTG |
| CasB10Rev | 2101 | CTTCTCACCGGTGTGCACCTGGCGCACGTG |
| CasB11Rev | 2102 | CTTCTCACCGGTGTGGGTGCGGGTGTGGGT |
| CasB12Rev | 2103 | CTTCTCACCGGTGTGCACGCGGTAGTGGGC |
| CasB13Rev | 2104 | CTTCTCACCGGTGTGCACGCGGTAGTGGGC |
| CasB14Rev | 2105 | CTTCTCACCGGTGTGGGTGCGCTCGTGCAG |
| CasB15Rev | 2106 | CTTCTCACCGGTGTGGCTGCGCTGGTGGCC |
| CasB16Rev | 2107 | CTTCTCACCGGTGTGGGTGCGCCAGTGGCT |
| CasC1Rev | 2108 | GATCGGATCCGCCGCCGTCCTTCTGGCGCAGGTGGATGC |
| | | GGATGTGGCGG |
| CasC2Rev | 2109 | GATCGGATCCGCCGCCGTCCTTCTGGCGCAGGTGGATCT |
| | | TGGTGTGGCGC |
| CasC3Rev | 2110 | GATCGGATCCGCCGCCGTCCTTCTGGCGCAGGTGGGTGC |
| | | GGTAGTGGCG |
| CasC4Rev | 2111 | GATCGGATCCGCCGCCGTCCTTCTGGCGCAGGTGCATGT |
| | | TGTGGTGGCGC |
| CasC5Rev | 2112 | GATCGGATCCGCCGCCGTCCTTCTGGCGCAGGTGGGTGC |
| | | GGTAGTGGCG |
| CasC6Rev | 2113 | GATCGGATCCGCCGCCGTCCTTCTGGCGCAGGTGGATGC |
| | | GCTTGTGGCGG |
| CasC7Rev | 2114 | GATCGGATCCGCCGCCGTCCTTCTGGCGCAGGTGCACGC |
| | | GCTTGTGGCG |
| CasC8Rev | 2115 | GATCGGATCCGCCGCCGTCCTTCTGGCGCAGGTGGGTGC |
| | | GCTGGTGGCG |
| ScaRev | 2116 | GTCATGCCATCCGTAAGATGC |
| GSFwd | 2117 | GGCGGATCCTATCCGTATGATGTG |
| Zif1Fwd | 2118 | AGAGAGAGAGAGATCTATGGCGGAAGAACGTCCGTATGC |
| | | GTGCCCGGTGGAAAG |
| Zif1Rev | 2119 | AGCCGGATCCCAAACACCGGTATGAATACGAATATGACG |
| | | GG |
| pETRev1 | 2120 | AGTGTAGCGGTCACGCTGC |

3-Finger Library Peptides

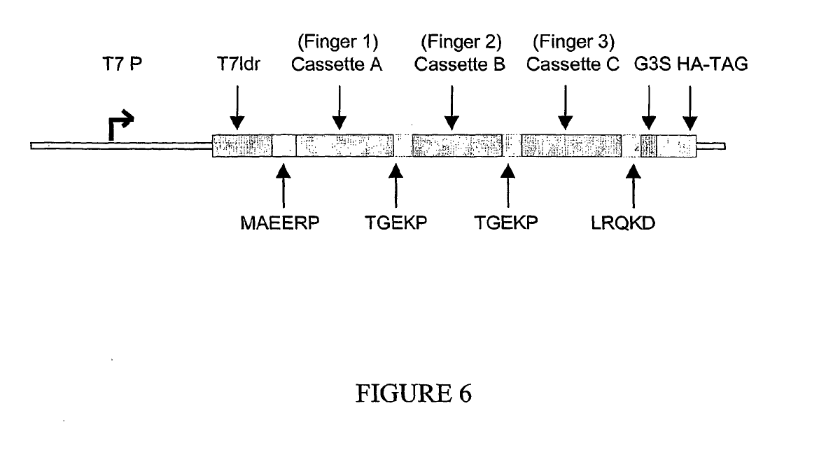

The 2 natural zinc finger modules for each construct are appended to the C-terminus of Zif268 finger 1 (as in Example 4, library 2). Hence, a plasmid construct containing Zif-?68 finger 1 and appropriate restriction sites for cloning of the two natural finger modules is also prepared. The construction and cloning procedure for the 3-finger libraries follows (see also FIG. 6).

(a) The plasmid pET23a/TZF-HA was assembled by PCR amplification of plasmid pTFZ-KOX (described in co-owned WO 01/53480) with primers AS1 and AS2. The sequences of these primers are as follows:

| | |
|---|---|
| AS1: | |
| CGATGGATCCATGGGAGAGAAGGCGCTGC | (SEQ ID NO: 2126) |
| AS2: | |
| GCGTAAAGCTTACGCATAATCCGGCACAT | (SEQ ID NO: 2127) |
| CATACGGATAAGAGCCGCCGCCGTCCTTC | |
| TGTCTTAAATGGATTT | |

The PCR product was gel purified and digested with BamHI and HindIII, then repurified and cloned into BamHI/Hind III-digested pET23a vector (Novagen), yielding pET23a/TFZ-HA. A number of clones were picked and sequenced to verify the correctness of the inserts.

(b) A fragment of approximately 1.2 kb is amplified from the vector pET23a/TFZ-HA, using the primers ScaRev and GSFwd (Table 10). This fragment contains the HA-epitope tag sequence (YPYDVPDYA* (SEQ ID NO:2122)) and part of the GGGS (SEQ ID NO:1988) linker sequence at the 5′ end. Additionally, the GSFwd primer adds a BamHI site at the extreme 5′ end. The ScaRev primer does not contain a restriction site, but a ScaI site from the vector is present approximately 40 bp downstream of the primer binding site. This fragment is cut with BamHI and ScaI and inserted into similarly cut pET23a.

(c) Zif268 finger 1 is then amplified using the PCR primers Zif1Fwd and Zif1Rev (Table 10), which add a BglII site at the 5′ end and both AgeI and BamHI sites at the 3′ end. This construct is then cut with BglII and BamHI and inserted into the vector construct made in step (b), which has been linearised with BamHI. At this stage the new construct, termed pET23aZif1HA is sequenced to find correctly oriented zinc finger inserts.

(d) Oligonucleotides encoding zinc finger modules for the C-terminus of the 3-finger constructs (cassette C) are amplified using the primers CasCxFor and CasCxRev (where x is 1 to 8, see Table 10). These cassettes are then digested with the restriction enzyme BamHI, and inserted into BamHI cut, dephosphorylated pET23aZif1HA. At this stage the new vector construct is not recircularised.

(e) Oligonucleotides encoding zinc finger modules for cassette B are amplified using primers CasBxFor and CasBxRev (where x is 9 to 16, see Table 10). These fragments are cut with the enzymes XmaI and AgeI, at 37° C. for 1-2 hours. The linear vector produced in stage (d) above, is also cut with AgeI and XmaI (as described), and dephosphorylated. Digested cassette B fragments are ligated into AgeI, XmaI cut vector, in the presence of the restriction enzymes AgeI and XmaI at room temperature for 16 hours. During this incubation incorrectly ligated fragments are re-digested and religated repeatedly, until the majority (or all) of the inserts are in the desired orientation. Correct 3-finger constructs have the assembly depicted in FIG. 6.

(f) Finally, 3-finger constructs are amplified from the ligated vector (produced in step (e)) using the primers pETFwd1 (Table 5) and pETRev1 (Table 10). 1 μl of each ligation mixture is amplified in a 10 μl (total volume) PCR reaction for 30 cycles. Alternatively, the ligated vector can be transformed into bacteria to produce samples containing single zinc finger clones.

The above procedure results in the majority of PCR products being the correct 3-finger constructs, so that any incorrect fragments will not significantly affect the selection protocol, and the PCR products can be used for screening without further processing. Alternatively, 3-finger PCR products may be purified from an agarose gel before use.

b. Screening of the Library Against 5′-GCGTGG-GCG3′

Members of the zinc finger library can be screened against the desired target site from a mixed population of clones, or from individual clones as described in Example 4, Protocol A or Protocol B (above), respectively. The target site for the screen is produced by annealing the oligonucleotides Zif.b site (AS154) and Zif site RC (AS155), as before. Template for protein expression is in each case made by PCR using primers pETFwd1 (Table 5) and pETRev1 (Table 10). 1 μl of each PCR reaction is used to express protein and screen for binding to the Zif site in the manner described in Example 4. The DNA corresponding to the samples giving the highest fluorescence signals is collected, purified from a 1% TAE-agarose gel, and sequenced to determine the sequence of the optimal binding 3-finger peptide.

### Example 6

### Reduced Human Zinc Finger Module Library for Universal DNA Recognition

A library system similar to that described in Example 5 can be constructed using zinc finger modules from databases such as those in Examples 1, 2 and 3 to select 2-finger units which bind any 2-finger (6 bp) recognition sequence. There are only 4096 (=4⁶) unique 6 bp sequences, therefore, a 2-finger library of natural zinc fingers (from specific animals, plants or fungi) can easily be constructed with enough variability to provide a specific 2-finger combination for optimal binding to any 6 bp target site. Again, to reduce the number of natural zinc finger modules that have to be constructed, a small selection of natural zinc finger modules (e.g., 3) are chosen for each 3 bp binding sequence (according to their predicted or determined recognition sequence). There are 64 (=4³) possible 3 bp binding sequences so in the first instance less than 200 (i.e. 192) natural zinc finger modules are constructed. These 200 zinc finger modules can be in either of 2 possible positions in the 2-finger construct, which gives approximately 40,000 (=200²) combinations of fingers to bind the 4096 possible 6 bp target sites. As in Example 5, these 2-finger units are attached to Zif268 finger 1 which acts as an anchor for DNA recognition.

a. Library Construction

The selected zinc finger modules are reverse translated from their amino acid sequences and synthesised as oligonucleotides. Double stranded zinc finger cassettes for both N-terminal and C-terminal fingers are created by PCR using primers specific for the relevant zinc finger module. Each zinc finger module is amplified in 2 separate reactions, as described in Example 5. The first PCR reaction uses primers which add TGEKP (SEQ ID NO:3) linker peptides and AgeI and XmaI restriction sites, to the 3′ and 5′ ends, respectively, to generate cassette B fragments. The second PCR reaction generates cassette C fragments by adding a TGEKP (SEQ ID NO:3) linker and an XmaI site at the 5′ end (this primer is the same as that used in cassette B production), and a sequence encoding the sequence LRQKDGGGS (SEQ ID NO:2125) and a BamHI restriction site at the 3′ end. The final constructs are similar to that represented in FIG. 6.

b. Library Selection

The collection of 3-finger zinc finger peptides produced above can be used to obtain specific domains for binding desired target sequences. Two exemplary approaches are described below.

i). Non-Cloning Selections.

A library constructed as described herein can be used to select optimal zinc finger domains for binding to any specified binding site. For instance, to select a peptide which binds the sequence 5′-GGA-TAA-3′, the binding site formed by annealing the oligonucleotides #1#5#6.b and #1#5#6 RC (Table 6, above), can be used as a target site (5′-GGA-TAA-GCG-3′). Selection of a zinc finger domain to bind such a target can be conducted, for example, in the manner described in Example 4. Briefly, the zinc finger library is diluted into 100 or more sub-libraries, which are screened as described above. The most active sub-libraries collected are further diluted to create much smaller sub-libraries, which are screened again, and so on. Following such a protocol, a library of 40,000 members can be fully screened and a high-affinity binder selected in just 3 rounds.

This selection procedure provides an extremely rapid method to select zinc finger peptides to bind any desired target site. The procedure also has the advantages of eliminating the need for cloning (as is required for methods such as phage display, see below), and is not limited by library size.

ii). Phage Library Selections

Zinc finger polypeptide phage display libraries are made and used to select clones encoding peptides that bind the desired nucleotide sequence, as described in co-owned WO 98/53057. An exemplary phage display library contains peptides which bind target sites with the sequence 5′-XXX-XXX-GCG-3′, where X can be any nucleotide. Hence, libraries of phage can be selected using the same target sites as described above. The selection protocol for zinc fingers displayed on phage is briefly described below.

Protocol

The selection protocol is adapted from that described in co-owned international patent application WO98/53057.

The 3-finger constructs of the present Example are PCR amplified using universal forward and reverse primers which contain sites for NotI and SfiI respectively (called NatPhageF and NatPhageR, respectively).

NatPhageF: (SEQ ID NO: 2128)
GCAACTGCGGCCCAGCCGGCCATGGCAGAGGAACGCCCGTATG
NatPhageR: (SEQ ID NO: 2129)
GAGTCATTCTGCGGCCGCGTCCTTCTGGCGCAGGTG

Backward PCR primers in addition introduce Met-Ala-Glu as the first three amino acid residues of the zinc finger polypeptides, and these are followed by the residues of the wild type or library zinc finger polypeptides as required. Cloning overhangs are produced by digestion with SfiI and NotI where necessary. Nucleic acid encoding zinc finger polypeptide fragments is ligated into similarly prepared Fd-Tet-SN vector. This is a derivative of fd-tet-DOG1 (Hoogenboom et al. (1991) Nucl. Acids Res. 19:4133-4137), in which a section of the pelB leader and a restriction site for the enzyme SfiI (underlined) have been added by site-directed mutagenesis using the oligonucleotide:

| | |
|---|---|
| 5′ CTCCTGCAGTTGGACCTGTGCCAT | (SEQ ID NO: 2130) |
| GGCCGGCTGGGCCGCATA | |
| GAATGGAACAACTAAAGC 3′ | |

that anneals in the region of the polylinker. Electrocompetent DH5α cells are transformed with recombinant vector in 200 ng aliquots, grown for 1 hour in 2xTY medium with 1% glucose, and plated on TYE containing 15 μg/ml tetracycline and 1% glucose.

To generate phage for selections, tetracycline resistant colonies are transferred from plates into 2xTY medium (16 g/litre Bacto tryptone, 10 g/litre Bacto yeast extract, 5 g/litre NaCl) containing 50 μM ZnCl₂ and 15 μg/nl tetracycline, and cultured overnight at 30° C. in a shaking incubator. Cleared culture supernatant containing phage particles is obtained by centrifuging at 300×g for 5 minutes.

Double stranded binding sites for use in selections are generated by annealing complementary oligonucleotides, one of which is biotinylated.

Biotinylated DNA target sites (1 pmol) are bound to streptavidin-coated wells (Roche). Phage supernatant solutions are diluted 1:10 in PBS selection buffer (PBS containing 50 μM ZnCl₂, 2% Marvel, 1% Tween, 20 μg/ml sonicated salmon sperm DNA, and 10-fold excess of competitor DNA), and 200 μl is applied to each well for 1 hour at 20° C. After this time, the wells are emptied and washed 18 times with PBS containing 50 μM ZnCl₂ and 1% Tween and 2 times in PBS containing 50 μM ZnCl₂. Retained phage are eluted in 100 μl 0.1M triethylamine and neutralised with an equal volume of 1M Tris (pH 7.4). Logarithmic-phase E. coli JM109 (100 μl) are infected with eluted phage (100 μl), and used to prepare phage supernatants for subsequent rounds of selection. After 4 rounds of selection, a ‘pool’ or ‘mini-population’ of phage is obtained, which bind the specified target sequence. These pools of phage can be stored at −70° C. for later use. Additionally, E. coli infected with these phage pools can be plated to obtain individual clones, which can be tested by ELISA for binding affinity and specificity to obtain the ‘best’ clone (see Example 9, Quality Control).

### Example 7

### Complete Human Zinc Finger Module Library for Universal DNA Recognition

An complete, or nearly complete, library containing all zinc finger sequences which bind a particular target site can be constructed using zinc finger modules to select 2-finger (or 3-finger) units which bind any 6 bp (or 9 bp) recognition sequence. Two exemplary methods for construction of such a library are described.

a. Oligonucleotide-Based Library Construction.

All zinc finger modules may be synthesised as a single stranded oligonucleotide, as described in Example 4. Zinc finger modules are made double stranded and TGEKP (SEQ ID NO:3) linkers added by PCR with 5′ and 3′ primers specific for each individual zinc finger module, to make cassettes. These cassettes can then be recombined, as described in Example 5, to make random or deliberate combinations of zinc finger modules comprising 2, 3, or more linked fingers.

b. PCR-Based Library Construction.

Zinc fingers proteins (especially of the Cys₂His₂ family) form the second most abundant family of proteins in the human genome. Furthermore, in nature, zinc finger modules are often linked by the canonical linker peptide TGEKP (SEQ ID NO:3), which begins immediately after the second zinc-coordinating histidine residue. Therefore, the peptide sequence HTGEKP (SEQ ID NO:2131) is commonly found between natural zinc finger modules. Because of this consensus sequence, it has been possible to clone natural zinc finger modules from the human genome (Becker, K. G., Nagel, J. W., Canning, R. D., Biddison, W. E., Ozato, K. & Drew, P. D. (1995) Hum. Mol. Genet. 4: 685-691; Bray, P., Lichter, P., Thiesen, H.-J., Ward, D. C. & Dawid, I. B. (1991) Proc. Natl. Acad. Sci. USA 88: 9563-9567), and the Arabidopsis genome (Meissner, R. & Michael, A. J. (1997) Plant Mol Biol 33: 615-624), using redundant primers for PCR. See also Pellegrino et al. (1991) Proc. Natl. Acad. Sci. USA 88:671-675. It is preferable to use genomic DNA or a genomic DNA (gDNA) library, rather than a cDNA library, because transcription factors, such as zinc finger proteins, are strongly regulated during the cell cycle, development and in response to extracellular signals. Hence, a cDNA library will probably not contain the majority of zinc finger proteins, and will be biased towards highly expressed proteins.

A suitable protocol for the PCR-extraction of zinc finger modules from human genomic DNA follows:

Genomic DNA is purified directly from human cells, or provided by a gDNA library. gDNA libraries are preferable as they are commercially available (for example from Clontech, ATCC, Stratagene etc) and can be easily manipulated. PCR to extract zinc finger modules can be conducted directly on purified gDNA, or the gDNA library can be screened for zinc fingers containing the HTGEKP (SEQ ID NO:2131) motif before carrying out PCR. To screen the gDNA library, any method known to one of skill in the art, e.g. colony hybridisation, can be used. Phage containing gDNA inserts are plated onto Escherichia coli XL-1 Blue bacterial lawns. At least 10⁶ phage plaques are transferred to replica filters and screened with, for example, a 27-mer ³²P-radiolabelled degenerate oligonucleotide, which anneals to the conserved linker region of zinc finger proteins and adjacent sequences. The sequence of a suitable degenerate probe (SEQ ID NO:2132), and the amino acid sequence (SEQ I) NO:2133) to which it corresponds is shown below.

C^{G}/T^{C}/G A^{T}/C^{C}/G CA^{C}/T AC^{C}/G GG^{C}/G GA^{G}/A AA^{G}/A CC^{C}/T T^{A}/T^{C}/T
R/L I/T/M H T G E K P Y/F

Hybridisation is performed, e.g., for 16 hours at 42-50 ° C., following which filters are washed 3-5 times, to remove non-specifically bound probe, in 0.2× standard saline citrate (SSC)/0.1% SDS. Filters are then subjected to autoradiography or phosphorimaging to determine positive plaques.

Positive plaques are picked into log-phase E. coli XL-1 Blue bacterial cultures and the phage are harvested for PCR. 1 μl phage supernatant is added to 49 μl PCR pre-mix, containing the oligonucleotide primers TGEKP for (SEQ ID NO:2134) and TGEKPrev (SEQ ID NO:2135) (shown below, annealing sequence in bold), and zinc finger modules are amplified by 30 cycles of PCR. TGEKP for (SEQ ID NO:2134) and TGEKPrev (SEQ ID NO:2135) also contain XbaI and EcoRI restriction sites (underlined), respectively. PCR products are-separated on 1.5% TAE-agarose gels and fragments of approximately 120 bp (corresponding to 1 zinc finger module plus flanking sequences) are purified, as described in Example 4. Additionally, fragments of approximately 220 bp, corresponding to natural 2-finger units, can also be collected and used. Such products can be digested with XbaI and EcoRI and cloned into a vector that has been digested so as to generate compatible ends, such as, for example, pcDNA3.1 (−) (Invitrogen) digested with EcoRI and XbaI. Such a vector pool can then be used as a source for natural 1- or 2-zinc finger modules, from which to construct 2- or 3-zinc finger peptides for selections as described above. Zinc finger modules for cassette B can be amplified from such vectors using the universal primers TGEKPXma (SEQ ID NO:2136) and TGEKPAge (SEQ ID NO:2137), which anneal to the conserved TGEKP (SEQ ID NO:3) linker regions and add restriction sites for the enzymes XmaI at the 5′ terminus and AgeI at the 3′ terminus, respectively (restriction sites underlined). Cassette C units can be amplified using the primer TGEKPXma (SEQ ID NO:2136) and TGEKPend (SEQ ID NO:2138), which adds a 3′ TRQKDGGGS (SEQ ID NO:2139) sequence incorporating a BamHI site (underlined, see below). Two- and 3-finger constructs can then be constructed and screened as described in the Examples above.

| | | |
|---|---|---|
| TGEKPfor: | TTAGTCTAGA^{C}/_{G}CA^{C}/_{T}AC^{C}/_{G}GG^{C}/_{G}GA^{G}/_{A}AA^{G}/_{A}CC | (SEQ ID NO: 2134) |
| TGEKPrev: | TACTGAATTC^{G}/_{A}GG^{C}/_{T}TT^{C}/_{T}TC^{G}/_{C}CC^{G}/_{C}GT^{G}/_{A}TG | (SEQ ID NO: 2135) |
| TGEKPXma: | TCTAGA^{C}/_{G}CA^{C}/_{T}CCCGGGGA^{G}/_{A}AA^{G}/_{A}CC | (SEQ ID NO: 2136) |
| TGEKPAge: | GAATTC^{G}/_{A}GG^{C}/_{T}TT^{C}/_{T}TCACCGGT^{G}/_{A}TG | (SEQ ID NO: 2137) |
| TGEKPend: | AGTGTGGTGGAATTC^{G}/_{A}GGGGATCCGCCGCCGTC^{C}/_{T}TT | (SEQ ID NO: 2138) |
| | ^{C}/_{T}TG^{G}/_{C}CG^{G}/_{C}GT^{G}/_{A}TG | |

### Example 8

### Microarray Analysis

Microarray analysis can also be used to determine the binding site specificity of 2- and 3-finger peptides. For example, a 3-zinc finger library, with finger 1 fixed as Zif268 finger one recognises the sequence 5′-XXX-XXX-GCG-3′, where X is any specified nucleotide. Hence, there are 4096 (=4⁶) unique binding sites for such a library. All 4096 of these sites can be arrayed onto a single glass slide, allowing a specified 2-finger peptide to be screened against every possible binding site at once. A suitable protocol for such an experiment is described in Martha L. Bulyk, Xiaohua Huang, Yen Choo, & George M. Church (Proc. Natl. Acad. Sci. USA: Vol.98, No.13, 7158-7163, Jun. 19, 2001) which is incorporated, by reference, in its entirety. See also co-owned WO 01/25417, the disclosure of which is hereby incorporated by reference in its entirety.

The amount of binding to each target sequence can be visualised and quantified using simple fluorescence measurements. For example, the zinc finger peptide can be expressed in vitro, or on the surface of phage. Isolated zinc finger peptides may contain an epitope tag for labelling purposes, whereas bound phage can be detected using a primary antibody against a phage coat protein, such as gVIII. A secondary antibody, such as one conjugated to R-phycoerythrin may be used to provide a visible signal when a: suitable substrate is applied.

### Example 9

### Quality Control

Particular 2- or 3-finger peptides can be screened to determine their specificity or affinity, as desired.

a. Phage ELISA Assay

Phage supernatants from Round 4 of selection (Example 6, supra) are used to infect E. coli JM109 bacteria, and grown to prepare fresh supernatants for zinc finger phage ELISA, using standard procedures as described previously (Choo, Y. & Klug, A. (1994) Proc. Natl. Acad. Sci. USA 91, 11163-11167; Choo, Y. & Klug, A. (1994) Proc. Natl. Acad. Sci. USA 91, 11168-11172). Briefly, 5′-biotinylated, positionally randomised oligonucleotide libraries, containing Zif268 binding site variants, are synthesised by annealing complimentary oligonucleotides as described supra. DNA libraries are added to streptavidin-coated ELISA wells (Boehringer-Mannheim) in PBS containing 50 μM ZnCl₂ (PBS/Zn). Phage solution (overnight bacterial culture supernatant diluted 1:10 in PBS/Zn containing 2% Marvel, 1% Tween and 20 μg/ml sonicated saimon sperm DNA) is applied to each well (50 μl/well). Binding is allowed to proceed for one hour at 20° C. Unbound phage are removed by washing 7 times with PBS/Zn containing 1% Tween, then 3 times with PBS/Zn. Bound phage are detected by ELISA using horseradish peroxidase-conjugated anti-M13 IgG (Pharmacia Biotech) and the colourimetric signal is quantitated using SOFTMAX 2.32 (Molecular Devices).

For rapid validation, the entire population of phage from Round 4 selection can be assayed in two ELISA wells: one containing the target DNA binding site, and one containing a control DNA binding site with between 1 and 5 base changes from the target sequence. A selection is deemed to be successful if the ELISA signal (representing DNA binding) is higher in the target well than in the control well.

The higher the signal measured above, the greater the population of specific binding clones. However, individual low values for such a procedure do not necessarily indicate a failure of the selection, as there may be individual high affinity/specificity clones within the round 4 phage population that may be masked by other non-specific clones. Nevertheless, this assay provides a quick profile of the overall quality of selection.

For a more detailed validation, individual phage clones are recovered from Round 4 by plating out infected bacterial colonies on agar. Fresh phage supernatants are prepared from these colonies and assayed by ELISA, as described above.

Finally, the coding sequence of individual zinc finger clones can be amplified by PCR using external primers complementary to phage sequence, and the PCR products are then sequenced to determine the amino acid sequence of the selected zinc fingers.

As an alternative, individual 3-finger peptides can be analysed by gel-shift assays or by microarray screening, as described infra. See also WO 00/41566, WO 00/42219 and WO 01/25417.

b. Gel-Shift Assay

Peptides are assayed using ³²P end-labelled synthetic oligonucleotide duplexes containing the appropriate binding site sequences.

DNA binding reactions contain the appropriate zinc-finger peptide, binding site and 1 μg competitor DNA (e.g., poly dI-dC or salmon sperm DNA) in a total volume of 10 μl, which contains: 20 mM Bis-tris propane (pH 7.0), 100 mM NaCl, 5 mM MgCl₂, 50 μM ZnCl₂, 5 mM DTT, 0.1 mg/ml BSA, 0.1% Nonidet P40. Incubations are performed at room temperature for 1 hour.

To determine the concentration of zinc finger peptide produced in the in vitro expression system, crude protein samples are used in gel-shift assays against a dilution series of the appropriate binding site. Binding site concentration is always well above the K_{d} of the peptide, but ranged from a higher concentration than the peptide (80 mM), at which all available peptide binds DNA, to a lower concentration (3-5 mM), at which all DNA is bound. Controls are carried out to ensure that binding sites are not shifted (i.e., bound) in the absence of zinc finger peptide. The reaction mixtures are then separated on a 7% native polyacrylamide gel. Radioactive signals are quantitated by PhosphorImager analysis to determine the amount of shifted binding site, and hence, the concentration of active zinc finger peptide.

Dissociation constants (K_{d}) are determined in parallel to the calculation of active peptide concentration. For determination of K_{d}, serial 3, 4 or 5-fold dilutions of crude peptide are made and incubated with radiolabelled binding site (10 pM-10 nM depending on the peptide), as above. Samples are run on 7% native polyacrylamide gels and the radioactive signals quantitated by PhosphorImager analysis. The data is then analysed according to linear transformation of the binding equation and plotted in CA-Cricket Graph III (Computer Associates Inc. NY) to generate the apparent dissociation constants. The K_{d} values reported are the average of at least two separate determinations.

C. Microarray Assay

Selected zinc finger domains can also be assayed for binding site specificity using the microarray analysis outlined in Example 8.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

1. A composite binding polypeptide comprising a first natural binding domain derived from a first natural binding polypeptide, and a second natural binding domain derived from a second natural binding polypeptide, wherein said first and second natural binding polypeptides may be the same or different; which polypeptide binds to a target, said target differing from the natural target of the both the first and the second binding polypeptides.

2. A composite polypeptide according to claim 1, wherein said first and second natural binding polypeptides are different polypeptides.

3. A composite polypeptide according to claim 1 comprising three or more natural binding domains.

4. A composite polypeptide according to claim 1 wherein the binding domains are nucleic acid binding domains.

5. A composite polypeptide according to claim 4, which is a nucleic acid binding polypeptide.

6. A composite polypeptide according to claim 4, which is a zinc finger polypeptide, and the natural binding domains are zinc finger domains.

7. A composite polypeptide according to claim 6, which comprises a Cys₂-His₂ zinc finger binding domain.

8. A composite polypeptide according to claim 6, which comprises a Cys₃-His zinc finger binding domain.

9. A composite polypeptide according to claim 1 which comprises 6 or more natural binding domains.

10. A composite polypeptide according to claim 9, wherein 6 natural binding domains are arranged in a 3×2 conformation, separated by linker sequences.

11. A composite polypeptide according to claim 1, further comprising a biological effector domain.

12. A library comprising multiple polynucleotides wherein each polynucleotide comprises a sequence that encodes a plurality of natural binding domains.

12. (canceled).

13. A library according to claim 12, wherein the binding domains comprise zinc finger binding domains and further wherein said zinc finger domains comprise a linker attached thereto.

14. A library according to claim 13, wherein the linker comprises the amino acid sequence TGEKP.

15. A method for selecting a binding polypeptide that binds to a target site, comprising:
(a) providing a library according to claim 12
(b) assembling two or more of said domains to form a composite polypeptide;
(c) screening said composite polypeptide against the target site in order to determine its ability to bind the target site.

16. A method according to claim 15, wherein the natural binding domains are zinc finger binding domains.

17. A method according to claim 15, wherein two or more composite polypeptides comprising two or more domains which are selected for binding to two or more target sites are combined to provide a composite polypeptide which binds to an aggregate binding site comprising the two or more target binding sites.

18. A method for designing a composite binding polypeptide, comprising:
(a) providing information defining a target site;
(b) selecting, from a database of natural binding domains, sequences of binding domains which are predicted to bind to the target site by the application of one or more rules which define target binding interactions for the binding domains; and
(c) displaying the polynucleotide sequences encoding the binding domains, and optionally assembling a polynucleotide encoding the binding polypeptide from a library according to claim 12.

19. A method according to claim 18, wherein the binding domains are zinc finger domains.

20. A method according to claim 19, wherein the zinc fingers recognize a nucleic acid triplet and domains are selected according to one or more of the following rules:
(a) if the 5′ base in the triplet is G, then position +6 in the α-helix is Arg; or position +6 is Ser or Thr and position ++2 is Asp;
(b) if the 5′ base in the triplet is A, then position +6 in the α-helix is Gln and ++2 is not Asp;
(c) if the 5′ base in the triplet is T, then position +6 in the α-helix is Ser or Thr and position ++2 is Asp;
(d) if the 5′ base in the triplet is C, then position +6 in the α-helix may be any amino acid, provided that position ++2 in the α-helix is not Asp;
(e) if the central base in the triplet is G, then position +3 in the α-helix is His;
(f) if the central base in the triplet is A, then position +3 in the α-helix is Asn; (g) if the central base in the triplet is T, then position +3 in the α-helix is Ala, Ser or Val; provided that if it is Ala, then one of the residues at −1 or +6 is a small residue;
(h) if the central base in the triplet is C, then position +3 in the α-helix is Ser, Asp, Glu, Leu, Thr or Val;
(i) if the 3′ base in the triplet is G, then position −1 in the α-helix is Arg;
(j) if the 3′ base in the triplet is A, then position −1 in the α-helix is Gln;
(k) if the 3′ base in the triplet is T, then position −1 in the α-helix is Asn or Gln; and
(l) if the 3′ base in the triplet is C, then position −1 in the α-helix is Asp.

21. A method according to claim 19, wherein the zinc fingers recognize a nucleic acid quadruplet and domains are selected according to one or more of the following rules:
(a) if base 4 in the quadruplet is G, then position +6 in the α-helix is Arg or Lys;
(b) if base 4 in the quadruplet is A, then position +6 in the α-helix is Glu, Asn or Val;
(c) if base 4 in the quadruplet is T, then position +6 in the α-helix is Ser, Thr, Val or Lys;
(d) if base 4 in the quadruplet is C, then position +6 in the α-helix is Ser, Thr, Val, Ala, Glu or Asn;
(e) if base 3 in the quadruplet is G, then position +3 in the α-helix is His;
(f) if base 3 in the quadruplet is A, then position +3 in the α-helix is Asn;
(g) if base 3 in the quadruplet is T, then position +3 in the α-helix is Ala, Ser or Val; provided that if it is Ala, then one of the residues at −1 or +6 is a small residue;
(h) if base 3 in the quadruplet is C, then position +3 in the α-helix is Ser, Asp, Glu, Leu, Thr or Val;
(i) if base 2 in the quadruplet is G, then position −1 in the α-helix is Arg;
(j) if base 2 in the quadruplet is A, then position −1 in the α-helix is Gln;
(k) if base 2 in the quadruplet is T, then position −1 in the α-helix is His or Thr;
(l) if base 2 in the quadruplet is C, then position −1 in the α-helix is Asp or His;
(m) if base 1 in the quadruplet is G, then position +2 is Glu;
(n) if base 1 in the quadruplet is A, then position +2 Arg or Gln;
(o) if base 1 in the quadruplet is C, then position +2 is Asn, Gln, Arg, His or Lys; and
(p) if base 1 in the quadruplet is T, then position +2 is Ser or Thr.

22. A method according to claim 19, wherein the zinc fingers recognize a nucleic acid quadruplet and domains are selected according to one or more of the following rules:
(a) if base 4 in the quadruplet is G, then position +6 in the α-helix is Arg; or position +6 is Ser or Thr and position ++2 is Asp;
(b) if base 4 in the quadruplet is A, then position +6 in the α-helix is Gln and ++2 is not Asp;
(c) if base 4 in the quadruplet is T, then position +6 in the α-helix is Ser or Thr and position ++2 is Asp;
(d) if base 4 in the quadruplet is C, then position +6 in the α-helix may be any amino acid, provided that position ++2 in the α-helix is not Asp;
(e) if base 3 in the quadruplet is G, then position +3 in the α-helix is His;
(f) if base 3 in the quadruplet is A, then position +3 in the α-helix is Asn;
(g) if base 3 in the quadruplet is T, then position +3 in the α-helix is Ala, Ser or Val; provided that if it is Ala, then one of the residues at −1 or +6 is a small residue;
(h) if base 3 in the quadruplet is C, then position +3 in the α-helix is Ser, Asp, Glu, Leu, Thr or Val;
(i) if base 2 in the quadruplet is G, then position −1 in the α-helix is Arg;
(j ) if base 2 in the quadruplet is A, then position −1 in the α-helix is Gln;
(k) if base 2 in the quadruplet is T, then position −1 in the α-helix is Asn or Gln;
(l) if base 2 in the quadruplet is C, then position −1 in the α-helix is Asp;
(m) if base 1 in the quadruplet is G, then position +2 is Asp;
(n) if base 1 in the quadruplet is A, then position +2 is not Asp;
(o) if base 1 in the quadruplet is C, then position +2 is not Asp; and
(p) if base 1 in the quadruplet is T, then position +2 is Ser or Thr.

23. The method of claim 18, further comprising the step of synthesizing a polynucleotide encoding the binding polypeptide.

24. A computer-implemented method for designing a zinc finger polypeptide, comprising the steps of
(a) providing a system comprising at least storage means for storing data relating to a library of zinc fingers; storage means for storing a rule table; means for inputting target nucleic acid sequence data; processing means for generating a result; and means for outputting the result;
(b) inputting sequence data for a target nucleic acid molecule;
(c) defining a first target zinc finger binding site in said nucleic acid molecule;
(d) interrogating the zinc finger library and rule table storage means, comparing zinc fingers to the target zinc finger binding site according to the rule table and selecting zinc finger data identifying a zinc finger capable of binding to said target site;
(e) defining at least one further target zinc finger binding site and repeating step (d); and
(f) outputting the selected zinc finger data.

25. A method according to claim 24, further comprising sending instructions to an automated chemical synthesis system to assemble a polynucleotide encoding a zinc finger polypeptide as defined by the zinc finger data obtained in (f).

26. A method according to claim 25, wherein the zinc finger polypeptide is tested for binding to the target site, and data from said testing is used to select, from a plurality of candidates, a zinc finger polypeptide that binds to the target site.

27. A method according to claim 25, wherein two or more zinc finger polypeptides are combined to form a zinc finger polypeptide capable of binding to an aggregate binding site comprising two or more target sites.
